SEQUENCE LISTING (305 sequences)

SEQ ID NO: 1 - DNA, 18 bp, synthetic construct
  source 1..18: mol_type = other DNA
cactctttcaacctggga

SEQ ID NO: 2 - DNA, 20 bp, synthetic construct
  source 1..20: mol_type = other DNA
ctaaggctccgcactgaggc

SEQ ID NO: 3 - DNA, 18 bp, synthetic construct
  source 1..18: mol_type = other DNA
atagaacagcaaatcttc

SEQ ID NO: 4 - DNA, 18 bp, synthetic construct
  source 1..18: mol_type = other DNA
ggcagaaacttttacttt

SEQ ID NO: 5 - DNA, 18 bp, synthetic construct
  source 1..18: mol_type = other DNA
aagatcattaacagcaat

SEQ ID NO: 6 - DNA, 18 bp, synthetic construct
  source 1..18: mol_type = other DNA
acaccatctgatgttgtt

SEQ ID NO: 7 - DNA, 18 bp, synthetic construct
  source 1..18: mol_type = other DNA
tattcattaccagccatc

SEQ ID NO: 8 - DNA, 18 bp, synthetic construct
  source 1..18: mol_type = other DNA
cttgtgtcaacaaaacaa

SEQ ID NO: 9 - DNA, 18 bp, synthetic construct
  source 1..18: mol_type = other DNA
cctgagaagtcatacatg

SEQ ID NO: 10 - DNA, 20 bp, synthetic construct
  source 1..20: mol_type = other DNA
aagatcattaacagcaatgg

SEQ ID NO: 11 - DNA, 20 bp, synthetic construct
  source 1..20: mol_type = other DNA
ctctttcaacctgggatcag

SEQ ID NO: 12 - DNA, 18 bp, synthetic construct
  source 1..18: mol_type = other DNA
cttacatagtcaaatttt

SEQ ID NO: 13 - DNA, 20 bp, synthetic construct
  source 1..20: mol_type = other DNA
gaaaggaacgaggcctcctg

SEQ ID NO: 14 - DNA, 20 bp, synthetic construct
  source 1..20: mol_type = other DNA
ccacaaatcgggactgaatt

SEQ ID NO: 15 - DNA, 20 bp, synthetic construct
  source 1..20: mol_type = other DNA
gtgtcaacaaaacaatgttg

SEQ ID NO: 16 - DNA, 20 bp, synthetic construct
  source 1..20: mol_type = other DNA
ccatacactctttcaacctg

SEQ ID NO: 17 - DNA, 20 bp, synthetic construct
  source 1..20: mol_type = other DNA
ctcggactcccgcagcggat

SEQ ID NO: 18 - DNA, 18 bp, synthetic construct
  source 1..18: mol_type = other DNA
attaacagcaatggcata

SEQ ID NO: 19 - DNA, 18 bp, synthetic construct
  source 1..18: mol_type = other DNA
catacactctttcaacct

SEQ ID NO: 20 - DNA, 18 bp, synthetic construct
  source 1..18: mol_type = other DNA
caagctaggtaacagacc

SEQ ID NO: 21 - DNA, 18 bp, synthetic construct
  source 1..18: mol_type = other DNA
gcaaagtgtctcaaatta

SEQ ID NO: 22 - DNA, 18 bp, synthetic construct
  source 1..18: mol_type = other DNA
tatagaacagcaaatctt

SEQ ID NO: 23 - DNA, 20 bp, synthetic construct
  source 1..20: mol_type = other DNA
ttttcacctgaggccaccag

SEQ ID NO: 24 - DNA, 18 bp, synthetic construct
  source 1..18: mol_type = other DNA
gctaggtaacagaccctg

SEQ ID NO: 25 - DNA, 18 bp, synthetic construct
  source 1..18: mol_type = other DNA
caagctaggtaacagacc

SEQ ID NO: 26 - DNA, 18 bp, synthetic construct
  source 1..18: mol_type = other DNA
atgttgtttgaagagtta

SEQ ID NO: 27 - DNA, 18 bp, synthetic construct
  source 1..18: mol_type = other DNA
caacttcaacatgaccta

SEQ ID NO: 28 - DNA, 20 bp, synthetic construct
  source 1..20: mol_type = other DNA
ctctttgccctcgctgttgc

SEQ ID NO: 29 - DNA, 18 bp, synthetic construct
  source 1..18: mol_type = other DNA
atacactctttcaacctg

SEQ ID NO: 30 - DNA, 18 bp, synthetic construct
  source 1..18: mol_type = other DNA
gatcattaacagcaatgg

SEQ ID NO: 31 - DNA, 20 bp, synthetic construct
  source 1..20: mol_type = other DNA
taacatatccatacactctt

SEQ ID NO: 32 - DNA, 18 bp, synthetic construct
  source 1..18: mol_type = other DNA
acttttcacctgaggcca

SEQ ID NO: 33 - DNA, 18 bp, synthetic construct
  source 1..18: mol_type = other DNA
aaagatctttgtctagca

SEQ ID NO: 34 - DNA, 20 bp, synthetic construct
  source 1..20: mol_type = other DNA
atacactctttcaacctggg

SEQ ID NO: 35 - DNA, 18 bp, synthetic construct
  source 1..18: mol_type = other DNA
tctgatgttgtttgaaga

SEQ ID NO: 36 - DNA, 20 bp, synthetic construct
  source 1..20: mol_type = other DNA
atagaacagcaaatcttcca

SEQ ID NO: 37 - DNA, 20 bp, synthetic construct
  source 1..20: mol_type = other DNA
ccagcacccaagattgagaa

SEQ ID NO: 38 - DNA, 20 bp, synthetic construct
  source 1..20: mol_type = other DNA
gttgtttgaagagttaatct

SEQ ID NO: 39 - DNA, 18 bp, synthetic construct
  source 1..18: mol_type = other DNA
cactcacttttcacctga

SEQ ID NO: 40 - DNA, 20 bp, synthetic construct
  source 1..20: mol_type = other DNA
ttatgaacaggtattttctc

SEQ ID NO: 41 - DNA, 18 bp, synthetic construct
  source 1..18: mol_type = other DNA
gtgtcaacaaaacaatgt

SEQ ID NO: 42 - DNA, 18 bp, synthetic construct
  source 1..18: mol_type = other DNA
attaaaggtttggcagaa

SEQ ID NO: 43 - DNA, 18 bp, synthetic construct
  source 1..18: mol_type = other DNA
atcaatgtgtgaggtaaa

SEQ ID NO: 44 - DNA, 20 bp, synthetic construct
  source 1..20: mol_type = other DNA
ctaggtaacagaccctgttt

SEQ ID NO: 45 - DNA, 18 bp, synthetic construct
  source 1..18: mol_type = other DNA
cagcatttaccataggat

SEQ ID NO: 46 - DNA, 18 bp, synthetic construct
  source 1..18: mol_type = other DNA
tgtcaacaaaacaatgtt

SEQ ID NO: 47 - DNA, 20 bp, synthetic construct
  source 1..20: mol_type = other DNA
tacactctttcaacctggga

SEQ ID NO: 48 - DNA, 20 bp, synthetic construct
  source 1..20: mol_type = other DNA
catatcgatgcacatattca

SEQ ID NO: 49 - DNA, 20 bp, synthetic construct
  source 1..20: mol_type = other DNA
gacaccatctgatgttgttt

SEQ ID NO: 50 - DNA, 18 bp, synthetic construct
  source 1..18: mol_type = other DNA
caattgtatagaacagca

SEQ ID NO: 51 - DNA, 20 bp, synthetic construct
  source 1..20: mol_type = other DNA
acctcaaataaatatttata

SEQ ID NO: 52 - DNA, 20 bp, synthetic construct
  source 1..20: mol_type = other DNA
cgcctcgcggagacactcac

SEQ ID NO: 53 - DNA, 18 bp, synthetic construct
  source 1..18: mol_type = other DNA
gggcagaaaccaccatta

SEQ ID NO: 54 - DNA, 20 bp, synthetic construct
  source 1..20: mol_type = other DNA
gctgctgctgctgctgctgc

SEQ ID NO: 55 - DNA, 18 bp, synthetic construct
  source 1..18: mol_type = other DNA
ctctttcaacctgggatc

SEQ ID NO: 56 - DNA, 18 bp, synthetic construct
  source 1..18: mol_type = other DNA
gtaattgggcagaaacca

SEQ ID NO: 57 - DNA, 18 bp, synthetic construct
  source 1..18: mol_type = other DNA
ggccagttgaggaatata

SEQ ID NO: 58 - DNA, 20 bp, synthetic construct
  source 1..20: mol_type = other DNA
tacactctttcaacctggga

SEQ ID NO: 59 - DNA, 18 bp, synthetic construct
  source 1..18: mol_type = other DNA
actctttcaacctgggat

SEQ ID NO: 60 - DNA, 20 bp, synthetic construct
  source 1..20: mol_type = other DNA
ctcacttttcacctgaggcc

SEQ ID NO: 61 - DNA, 18 bp, synthetic construct
  source 1..18: mol_type = other DNA
ccatctgatgttgtttga

SEQ ID NO: 62 - DNA, 18 bp, synthetic construct
  source 1..18: mol_type = other DNA
ttggtcaccaaacatatc

SEQ ID NO: 63 - DNA, 20 bp, synthetic construct
  source 1..20: mol_type = other DNA
ttctaaatgcttgtgtcaac

SEQ ID NO: 64 - DNA, 20 bp, synthetic construct
  source 1..20: mol_type = other DNA
gctgctgcgcgctaggattg

SEQ ID NO: 65 - DNA, 20 bp, synthetic construct
  source 1..20: mol_type = other DNA
ctctgtccatctactgtaca

SEQ ID NO: 66 - DNA, 18 bp, synthetic construct
  source 1..18: mol_type = other DNA
tctttcaacctgggatca

SEQ ID NO: 67 - DNA, 18 bp, synthetic construct
  source 1..18: mol_type = other DNA
agatctttgtctagcatg

SEQ ID NO: 68 - DNA, 18 bp, synthetic construct
  source 1..18: mol_type = other DNA
aagatctttgtctagcat

SEQ ID NO: 69 - DNA, 20 bp, synthetic construct
  source 1..20: mol_type = other DNA
ctacttgggtgtggttccgg

SEQ ID NO: 70 - DNA, 18 bp, synthetic construct
  source 1..18: mol_type = other DNA
tggacagactgtacaaca

SEQ ID NO: 71 - DNA, 18 bp, synthetic construct
  source 1..18: mol_type = other DNA
gatctttgtctagcatga

SEQ ID NO: 72 - DNA, 18 bp, synthetic construct
  source 1..18: mol_type = other DNA
tttaagttaagatagaaa

SEQ ID NO: 73 - DNA, 18 bp, synthetic construct
  source 1..18: mol_type = other DNA
tttcatataactcatcaa

SEQ ID NO: 74 - DNA, 18 bp, synthetic construct
  source 1..18: mol_type = other DNA
tgtatagaacagcaaatc

SEQ ID NO: 75 - DNA, 20 bp, synthetic construct
  source 1..20: mol_type = other DNA
aacagacacaccccacaaat

SEQ ID NO: 76 - DNA, 18 bp, synthetic construct
  source 1..18: mol_type = other DNA
ttaaaaagatctttgtct

SEQ ID NO: 77 - DNA, 20 bp, synthetic construct
  source 1..20: mol_type = other DNA
agcattatttactccttgct

SEQ ID NO: 78 - DNA, 20 bp, synthetic construct
  source 1..20: mol_type = other DNA
catataactcatcaatgtgt

SEQ ID NO: 79 - DNA, 18 bp, synthetic construct
  source 1..18: mol_type = other DNA
ttgaagagttaatcttaa

SEQ ID NO: 80 - DNA, 20 bp, synthetic construct
  source 1..20: mol_type = other DNA
ctgctgctgctgctgctgct

SEQ ID NO: 81 - DNA, 20 bp, synthetic construct
  source 1..20: mol_type = other DNA
taaatgaagaaacattctga

SEQ ID NO: 82 - DNA, 18 bp, synthetic construct
  source 1..18: mol_type = other DNA
atagcatagcaaatcacc

SEQ ID NO: 83 - DNA, 20 bp, synthetic construct
  source 1..20: mol_type = other DNA
catcatgccgccgggtccgt

SEQ ID NO: 84 - DNA, 18 bp, synthetic construct
  source 1..18: mol_type = other DNA
aagacataaagtcaggaa

SEQ ID NO: 85 - DNA, 20 bp, synthetic construct
  source 1..20: mol_type = other DNA
agacactcacttttcacctg

SEQ ID NO: 86 - DNA, 20 bp, synthetic construct
  source 1..20: mol_type = other DNA
agatctttgtctagcatgac

SEQ ID NO: 87 - DNA, 20 bp, synthetic construct
  source 1..20: mol_type = other DNA
cgcggagacactcacttttc

SEQ ID NO: 88 - DNA, 20 bp, synthetic construct
  source 1..20: mol_type = other DNA
ttgtccttcagcaattgtat

SEQ ID NO: 89 - DNA, 20 bp, synthetic construct
  source 1..20: mol_type = other DNA
tgttgaatcttagtccactc

SEQ ID NO: 90 - DNA, 18 bp, synthetic construct
  source 1..18: mol_type = other DNA
gcaaatcaccattaaagg

SEQ ID NO: 91 - DNA, 18 bp, synthetic construct
  source 1..18: mol_type = other DNA
ccctgagaagtcatacat

SEQ ID NO: 92 - DNA, 18 bp, synthetic construct
  source 1..18: mol_type = other DNA
ggagacactcacttttca

SEQ ID NO: 93 - DNA, 20 bp, synthetic construct
  source 1..20: mol_type = other DNA
acactctttcaacctgggat

SEQ ID NO: 94 - DNA, 18 bp, synthetic construct
  source 1..18: mol_type = other DNA
gagacactcacttttcac

SEQ ID NO: 95 - DNA, 18 bp, synthetic construct
  source 1..18: mol_type = other DNA
gtatagcatagcaaatca

SEQ ID NO: 96 - DNA, 20 bp, synthetic construct
  source 1..20: mol_type = other DNA
gtggttccggctcggactcc

SEQ ID NO: 97 - DNA, 20 bp, synthetic construct
  source 1..20: mol_type = other DNA
catacactctttcaacctgg

SEQ ID NO: 98 - DNA, 18 bp, synthetic construct
  source 1..18: mol_type = other DNA
atctttgtctagcatgac

SEQ ID NO: 99 - DNA, 20 bp, synthetic construct
  source 1..20: mol_type = other DNA
catacactctttcaacctgg

SEQ ID NO: 100 - DNA, 18 bp, synthetic construct
  source 1..18: mol_type = other DNA
gtcaccaaacatatctta

SEQ ID NO: 101 - DNA, 20 bp, synthetic construct
  source 1..20: mol_type = other DNA
tggccagttgaggaatataa

SEQ ID NO: 102 - DNA, 20 bp, synthetic construct
  source 1..20: mol_type = other DNA
ccatacactctttcaacctg

SEQ ID NO: 103 - DNA, 18 bp, synthetic construct
  source 1..18: mol_type = other DNA
ccaagatcattaacagca

SEQ ID NO: 104 - DNA, 18 bp, synthetic construct
  source 1..18: mol_type = other DNA
ctgatgttgtttgaagag

SEQ ID NO: 105 - DNA, 18 bp, synthetic construct
  source 1..18: mol_type = other DNA
ttatgtggtttatctgta

SEQ ID NO: 106 - DNA, 18 bp, synthetic construct
  source 1..18: mol_type = other DNA
ccaacatattcattacca

SEQ ID NO: 107 - DNA, 18 bp, synthetic construct
  source 1..18: mol_type = other DNA
gattatgtggtttatctg

SEQ ID NO: 108 - DNA, 20 bp, synthetic construct
  source 1..20: mol_type = other DNA
cttactgtaataaatttatg

SEQ ID NO: 109 - DNA, 18 bp, synthetic construct
  source 1..18: mol_type = other DNA
agtgtctcaaattatcat

SEQ ID NO: 110 - DNA, 20 bp, synthetic construct
  source 1..20: mol_type = other DNA
cactctttcaacctgggatc

SEQ ID NO: 111 - DNA, 18 bp, synthetic construct
  source 1..18: mol_type = other DNA
ttgggcagaaaccaccat

SEQ ID NO: 112 - DNA, 20 bp, synthetic construct
  source 1..20: mol_type = other DNA
ataaacttacctctgtccat

SEQ ID NO: 113 - DNA, 18 bp, synthetic construct
  source 1..18: mol_type = other DNA
gctagttgttcaagcaac

SEQ ID NO: 114 - DNA, 20 bp, synthetic construct
  source 1..20: mol_type = other DNA
tgattcacaagtcacttcaa

SEQ ID NO: 115 - DNA, 20 bp, synthetic construct
  source 1..20: mol_type = other DNA
aaagatctttgtctagcatg

SEQ ID NO: 116 - DNA, 18 bp, synthetic construct
  source 1..18: mol_type = other DNA
accatctgatgttgtttg

SEQ ID NO: 117 - DNA, 20 bp, synthetic construct
  source 1..20: mol_type = other DNA
aaagcattacctttcctcca

SEQ ID NO: 118 - DNA, 18 bp, synthetic construct
  source 1..18: mol_type = other DNA
gcaattgtatagaacagc

SEQ ID NO: 119 - DNA, 20 bp, synthetic construct
  source 1..20: mol_type = other DNA
tagtgaagtcacaacaattg

SEQ ID NO: 120 - DNA, 20 bp, synthetic construct
  source 1..20: mol_type = other DNA
ccgcgaccggttctcttcgc

SEQ ID NO: 121 - DNA, 20 bp, synthetic construct
  source 1..20: mol_type = other DNA
attacctttcctccagactg

SEQ ID NO: 122 - DNA, 20 bp, synthetic construct
  source 1..20: mol_type = other DNA
gtaacagaccctgttttatt

SEQ ID NO: 123 - DNA, 18 bp, synthetic construct
  source 1..18: mol_type = other DNA
ccaagctaggtaacagac

SEQ ID NO: 124 - DNA, 18 bp, synthetic construct
  source 1..18: mol_type = other DNA
agttaatcttaacatatc

SEQ ID NO: 125 - DNA, 18 bp, synthetic construct
  source 1..18: mol_type = other DNA
atcaccattaaaggtttg

SEQ ID NO: 126 - DNA, 18 bp, synthetic construct
  source 1..18: mol_type = other DNA
tactgtaataaatttatg

SEQ ID NO: 127 - DNA, 20 bp, synthetic construct
  source 1..20: mol_type = other DNA
acactctttcaacctgggat

SEQ ID NO: 128 - DNA, 18 bp, synthetic construct
  source 1..18: mol_type = other DNA
ttgtgtcaacaaaacaat

SEQ ID NO: 129 - DNA, 18 bp, synthetic construct
  source 1..18: mol_type = other DNA
ccatacactctttcaacc

SEQ ID NO: 130 - DNA, 20 bp, synthetic construct
  source 1..20: mol_type = other DNA
acaaactttcttctaaatgc

SEQ ID NO: 131 - DNA, 18 bp, synthetic construct
  source 1..18: mol_type = other DNA
tttagcactttcatataa

SEQ ID NO: 132 - DNA, 18 bp, synthetic construct
  source 1..18: mol_type = other DNA
tagcatgacaccatctga

SEQ ID NO: 133 - DNA, 18 bp, synthetic construct
  source 1..18: mol_type = other DNA
agacataaagtcaggaat

SEQ ID NO: 134 - DNA, 20 bp, synthetic construct
  source 1..20: mol_type = other DNA
tagatttgagtgcctgtttt

SEQ ID NO: 135 - DNA, 18 bp, synthetic construct
  source 1..18: mol_type = other DNA
gacaccatctgatgttgt

SEQ ID NO: 136 - DNA, 20 bp, synthetic construct
  source 1..20: mol_type = other DNA
gtcccgcagcatccccgagc

SEQ ID NO: 137 - DNA, 18 bp, synthetic construct
  source 1..18: mol_type = other DNA
taggattatgtggtttat

SEQ ID NO: 138 - DNA, 18 bp, synthetic construct
  source 1..18: mol_type = other DNA
atcattaacagcaatggc

SEQ ID NO: 139 - DNA, 18 bp, synthetic construct
  source 1..18: mol_type = other DNA
ctttcaacctgggatcag

SEQ ID NO: 140 - DNA, 18 bp, synthetic construct
  source 1..18: mol_type = other DNA
gtcaacaaaacaatgttg

SEQ ID NO: 141 - DNA, 18 bp, synthetic construct
  source 1..18: mol_type = other DNA
caccatctgatgttgttt

SEQ ID NO: 142 - DNA, 20 bp, synthetic construct
  source 1..20: mol_type = other DNA
tgctgctgctgctgctgcgc

SEQ ID NO: 143 - DNA, 20 bp, synthetic construct
  source 1..20: mol_type = other DNA
gcaatcctgtagatttgagt

SEQ ID NO: 144 - DNA, 18 bp, synthetic construct
  source 1..18: mol_type = other DNA
gcactttcatataactca

SEQ ID NO: 145 - DNA, 20 bp, synthetic construct
  source 1..20: mol_type = other DNA
atccccgagcctcgcagccg

SEQ ID NO: 146 - DNA, 18 bp, synthetic construct
  source 1..18: mol_type = other DNA
gccctgagaagtcataca

SEQ ID NO: 147 - DNA, 18 bp, synthetic construct
  source 1..18: mol_type = other DNA
agatctttgtctagcatg

SEQ ID NO: 148 - DNA, 20 bp, synthetic construct
  source 1..20: mol_type = other DNA
cttacctctgtccatctact

SEQ ID NO: 149 - DNA, 18 bp, synthetic construct
  source 1..18: mol_type = other DNA
acactctttcaacctggg

SEQ ID NO: 150 - DNA, 20 bp, synthetic construct
  source 1..20: mol_type = other DNA
attgagaacaaaaaatacct

SEQ ID NO: 151 - DNA, 18 bp, synthetic construct
  source 1..18: mol_type = other DNA
tatagaacagcaaatctt

SEQ ID NO: 152 - DNA, 18 bp, synthetic construct
  source 1..18: mol_type = other DNA
caaatcaccattaaaggt

SEQ ID NO: 153 - DNA, 18 bp, synthetic construct
  source 1..18: mol_type = other DNA
ttaccatcttcattcaaa

SEQ ID NO: 154 - DNA, 20 bp, synthetic construct
  source 1..20: mol_type = other DNA
cgcagcggatgcgggtgctg

SEQ ID NO: 155 - DNA, 18 bp, synthetic construct
  source 1..18: mol_type = other DNA
ccagcatttaccatagga

SEQ ID NO: 156 - DNA, 18 bp, synthetic construct
  source 1..18: mol_type = other DNA
gggaaagggtttactttg

SEQ ID NO: 157 - DNA, 18 bp, synthetic construct
  source 1..18: mol_type = other DNA
caagatcattaacagcaa

SEQ ID NO: 158 - DNA, 20 bp, synthetic construct
  source 1..20: mol_type = other DNA
tgtaataaatttatgaacag

SEQ ID NO: 159 - DNA, 18 bp, synthetic construct
  source 1..18: mol_type = other DNA
agaaaccaccattagcca

SEQ ID NO: 160 - DNA, 20 bp, synthetic construct
  source 1..20: mol_type = other DNA
atcaatgtgtgaggtaaaag

SEQ ID NO: 161 - DNA, 20 bp, synthetic construct
  source 1..20: mol_type = other DNA
gcggtgagatgacagaagag

SEQ ID NO: 162 - DNA, 18 bp, synthetic construct
  source 1..18: mol_type = other DNA
cttaccatcttcattcaa

SEQ ID NO: 163 - DNA, 18 bp, synthetic construct
  source 1..18: mol_type = other DNA
gaacgcggtctgtggtgg

SEQ ID NO: 164 - DNA, 20 bp, synthetic construct
  source 1..20: mol_type = other DNA
tgaagaaacattctgaaact

SEQ ID NO: 165 - DNA, 18 bp, synthetic construct
  source 1..18: mol_type = other DNA
ccagccatcttattcaaa

SEQ ID NO: 166 - DNA, 20 bp, synthetic construct
  source 1..20: mol_type = other DNA
gtgtcaacaaaacaatgttg

SEQ ID NO: 167 - DNA, 20 bp, synthetic construct
  source 1..20: mol_type = other DNA
gagttaatcttaacatatcc

SEQ ID NO: 168 - DNA, 20 bp, synthetic construct
  source 1..20: mol_type = other DNA
tcaacctgggatcagacgtt

SEQ ID NO: 169 - DNA, 18 bp, synthetic construct
  source 1..18: mol_type = other DNA
tttacacaggactgaaga

SEQ ID NO: 170 - DNA, 20 bp, synthetic construct
  source 1..20: mol_type = other DNA
gacagaagaggaaagggcag

SEQ ID NO: 171 - DNA, 18 bp, synthetic construct
  source 1..18: mol_type = other DNA
gttgaggaatataatctg

SEQ ID NO: 172 - DNA, 20 bp, synthetic construct
  source 1..20: mol_type = other DNA
aagacataaagtcaggaatc

SEQ ID NO: 173 - DNA, 18 bp, synthetic construct
  source 1..18: mol_type = other DNA
ttcacctgaggccaccag

SEQ ID NO: 174 - DNA, 18 bp, synthetic construct
  source 1..18: mol_type = other DNA
gcacttacatagtcaaat

SEQ ID NO: 175 - DNA, 18 bp, synthetic construct
  source 1..18: mol_type = other DNA
tttcaacctgggatcaga

SEQ ID NO: 176 - DNA, 18 bp, synthetic construct
  source 1..18: mol_type = other DNA
taactcatcaatgtgtga

SEQ ID NO: 177 - DNA, 20 bp, synthetic construct
  source 1..20: mol_type = other DNA
ctctttcaacctgggatcag

SEQ ID NO: 178 - DNA, 20 bp, synthetic construct
  source 1..20: mol_type = other DNA
aggcctcctgcgcctcgcgg

SEQ ID NO: 179 - DNA, 20 bp, synthetic construct
  source 1..20: mol_type = other DNA
gcgggtgctgctgctgctgc

SEQ ID NO: 180 - DNA, 20 bp, synthetic construct
  source 1..20: mol_type = other DNA
caaaaaatacctcaaataaa

SEQ ID NO: 181 - DNA, 18 bp, synthetic construct
  source 1..18: mol_type = other DNA
agatcattaacagcaatg

SEQ ID NO: 182 - DNA, 20 bp, synthetic construct
  source 1..20: mol_type = other DNA
ttctcttcgctccgcctaag

SEQ ID NO: 183 - DNA, 18 bp, synthetic construct
  source 1..18: mol_type = other DNA
ttggaaagaaatgaatag

SEQ ID NO: 184 - DNA, 20 bp, synthetic construct
  source 1..20: mol_type = other DNA
aacaatgttgctctgaacac

SEQ ID NO: 185 - DNA, 18 bp, synthetic construct
  source 1..18: mol_type = other DNA
tacactctttcaacctgg

SEQ ID NO: 186 - DNA, 20 bp, synthetic construct
  source 1..20: mol_type = other DNA
aggaatataatctgcaacct

SEQ ID NO: 187 - DNA, 20 bp, synthetic construct
  source 1..20: mol_type = other DNA
ctactgtacaaacagacaca

SEQ ID NO: 188 - DNA, 20 bp, synthetic construct
  source 1..20: mol_type = other DNA
taccataggattatgtggtt

SEQ ID NO: 189 - DNA, 18 bp, synthetic construct
  source 1..18: mol_type = other DNA
tgtttgaagagttaatct

SEQ ID NO: 190 - DNA, 18 bp, synthetic construct
  source 1..18: mol_type = other DNA
aacttactgtaataaatt

SEQ ID NO: 191 - DNA, 18 bp, synthetic construct
  source 1..18: mol_type = other DNA
tgtgtcaacaaaacaatg

SEQ ID NO: 192 - DNA, 18 bp, synthetic construct
  source 1..18: mol_type = other DNA
aattgtatagaacagcaa

SEQ ID NO: 193 - DNA, 18 bp, synthetic construct
  source 1..18: mol_type = other DNA
ttgtatagaacagcaaat

SEQ ID NO: 194 - DNA, 18 bp, synthetic construct
  source 1..18: mol_type = other DNA
acaacttcaacatgacct

SEQ ID NO: 195 - DNA, 18 bp, synthetic construct
  source 1..18: mol_type = other DNA
aattatcatagttatgga

SEQ ID NO: 196 - DNA, 20 bp, synthetic construct
  source 1..20: mol_type = other DNA
gagaagtcatacatgccaca

SEQ ID NO: 197 - DNA, 18 bp, synthetic construct
  source 1..18: mol_type = other DNA
tgatgttgtttgaagagt

SEQ ID NO: 198 - DNA, 18 bp, synthetic construct
  source 1..18: mol_type = other DNA
gcttaccctttgatcacc

SEQ ID NO: 199 - DNA, 20 bp, synthetic construct
  source 1..20: mol_type = other DNA
gtattttctcttgtccttca

SEQ ID NO: 200 - DNA, 20 bp, synthetic construct
  source 1..20: mol_type = other DNA
gcaattgtatagaacagcaa

SEQ ID NO: 201 - DNA, 18 bp, synthetic construct
  source 1..18: mol_type = other DNA
tcattaccagccatctta

SEQ ID NO: 202 - DNA, 20 bp, synthetic construct
  source 1..20: mol_type = other DNA
taccataggattatgtggtt

SEQ ID NO: 203 - DNA, 20 bp, synthetic construct
  source 1..20: mol_type = other DNA
actagaacccatttctttag

SEQ ID NO: 204 - DNA, 18 bp, synthetic construct
  source 1..18: mol_type = other DNA
atgtggtttatctgtaaa

SEQ ID NO: 205 - DNA, 20 bp, synthetic construct
  source 1..20: mol_type = other DNA
tgaacacatctgtagaatgt

SEQ ID NO: 206 - DNA, 20 bp, synthetic construct
  source 1..20: mol_type = other DNA
ccgggcgtgggcggccggtt

SEQ ID NO: 207 - DNA, 20 bp, synthetic construct
  source 1..20: mol_type = other DNA
atcagacgttcgcaatcctg

SEQ ID NO: 208 - DNA, 18 bp, synthetic construct
  source 1..18: mol_type = other DNA
ttaccatcttcattcaaa

SEQ ID NO: 209 - DNA, 20 bp, synthetic construct
  source 1..20: mol_type = other DNA
ttagcactttcatataactc

SEQ ID NO: 210 - DNA, 20 bp, synthetic construct
  source 1..20: mol_type = other DNA
aggccaccagctctttgccc

SEQ ID NO: 211 - DNA, 18 bp, synthetic construct
  source 1..18: mol_type = other DNA
atctttgtctagcatgac

SEQ ID NO: 212 - DNA, 18 bp, synthetic construct
  source 1..18: mol_type = other DNA
tcattaacagcaatggca

SEQ ID NO: 213 - DNA, 20 bp, synthetic construct
  source 1..20: mol_type = other DNA
agaacagcaaatcttccaag

SEQ ID NO: 214 - DNA, 20 bp, synthetic construct
  source 1..20: mol_type = other DNA
actctttcaacctgggatca

SEQ ID NO: 215 - DNA, 20 bp, synthetic construct
  source 1..20: mol_type = other DNA
cccgggccccgaaaggaacg

SEQ ID NO: 216 - DNA, 18 bp, synthetic construct
  source 1..18: mol_type = other DNA
aaattatcatagttatgg

SEQ ID NO: 217 - DNA, 18 bp, synthetic construct
  source 1..18: mol_type = other DNA
acacagagaaacaagatc

SEQ ID NO: 218 - DNA, 18 bp, synthetic construct
  source 1..18: mol_type = other DNA
catctgatgttgtttgaa

SEQ ID NO: 219 - DNA, 18 bp, synthetic construct
  source 1..18: mol_type = other DNA
attgtatagaacagcaaa

SEQ ID NO: 220 - DNA, 18 bp, synthetic construct
  source 1..18: mol_type = other DNA
tgactcaggtgacacttt

SEQ ID NO: 221 - DNA, 20 bp, synthetic construct
  source 1..20: mol_type = other DNA
atcttccaagctaggtaaca

SEQ ID NO: 222 - DNA, 18 bp, synthetic construct
  source 1..18: mol_type = other DNA
catttaccataggattat

SEQ ID NO: 223 - DNA, 18 bp, synthetic construct
  source 1..18: mol_type = other DNA
tttcaccagtaattgggc

SEQ ID NO: 224 - DNA, 20 bp, synthetic construct
  source 1..20: mol_type = other DNA
accatctgatgttgtttgaa

SEQ ID NO: 225 - DNA, 20 bp, synthetic construct
  source 1..20: mol_type = other DNA
acattctgaaactagaaccc

SEQ ID NO: 226 - DNA, 20 bp, synthetic construct
  source 1..20: mol_type = other DNA
gtcaggaatcacaaactttc

SEQ ID NO: 227 - DNA, 20 bp, synthetic construct
  source 1..20: mol_type = other DNA
ctagcatgacaccatctgat

SEQ ID NO: 228 - DNA, 18 bp, synthetic construct
  source 1..18: mol_type = other DNA
ttgactcaggtgacactt

SEQ ID NO: 229 - DNA, 20 bp, synthetic construct
  source 1..20: mol_type = other DNA
ggactgaatttggccagttg

SEQ ID NO: 230 - DNA, 18 bp, synthetic construct
  source 1..18: mol_type = other DNA
atctgatgttgtttgaag

SEQ ID NO: 231 - DNA, 18 bp, synthetic construct
  source 1..18: mol_type = other DNA
ctctttcaacctgggatc

SEQ ID NO: 232 - DNA, 20 bp, synthetic construct
  source 1..20: mol_type = other DNA
caaacgcgtccgtctccccg

SEQ ID NO: 233 - DNA, 20 bp, synthetic construct
  source 1..20: mol_type = other DNA
atacactctttcaacctggg

SEQ ID NO: 234 - DNA, 20 bp, synthetic construct
  source 1..20: mol_type = other DNA
ttgaaagattaaatgaagaa

SEQ ID NO: 235 - DNA, 20 bp, synthetic construct
  source 1..20: mol_type = other DNA
tccgcctaaggctccgcact

SEQ ID NO: 236 - DNA, 20 bp, synthetic construct
  source 1..20: mol_type = other DNA
aaagatctttgtctagcatg

SEQ ID NO: 237 - DNA, 18 bp, synthetic construct
  source 1..18: mol_type = other DNA
cattaacagcaatggcat

SEQ ID NO: 238 - DNA, 18 bp, synthetic construct
  source 1..18: mol_type = other DNA
tatactacagttgtagag

SEQ ID NO: 239 - DNA, 18 bp, synthetic construct
  source 1..18: mol_type = other DNA
gcatagcaaatcaccatt

SEQ ID NO: 240 - DNA, 18 bp, synthetic construct
  source 1..18: mol_type = other DNA
catacactctttcaacct

SEQ ID NO: 241 - DNA, 18 bp, synthetic construct
  source 1..18: mol_type = other DNA
tgacaccatctgatgttg

SEQ ID NO: 242 - DNA, 20 bp, synthetic construct
  source 1..20: mol_type = other DNA
aagatcattaacagcaatgg

SEQ ID NO: 243 - DNA, 20 bp, synthetic construct
  source 1..20: mol_type = other DNA
cactctttcaacctgggatc

SEQ ID NO: 244 - DNA, 20 bp, synthetic construct
  source 1..20: mol_type = other DNA
ggaggatgctccgggcgtgg

SEQ ID NO: 245 - DNA, 18 bp, synthetic construct
  source 1..18: mol_type = other DNA
ttaccataggattatgtg

SEQ ID NO: 246 - DNA, 20 bp, synthetic construct
  source 1..20: mol_type = other DNA
ctcgcagccgcatcatgccg

SEQ ID NO: 247 - DNA, 18 bp, synthetic construct
  source 1..18: mol_type = other DNA
tgaagagttaatcttaac

SEQ ID NO: 248 - DNA, 20 bp, synthetic construct
  source 1..20: mol_type = other DNA
agaacccatttctttagctt

SEQ ID NO: 249 - DNA, 20 bp, synthetic construct
  source 1..20: mol_type = other DNA
atcttatccagaggaggaga

SEQ ID NO: 250 - DNA, 20 bp, synthetic construct
  source 1..20: mol_type = other DNA
cgcaatcctgtagatttgag

SEQ ID NO: 251 - DNA, 20 bp, synthetic construct
  source 1..20: mol_type = other DNA
gaaagggcagctacttgggt

SEQ ID NO: 252 - DNA, 20 bp, synthetic construct
  source 1..20: mol_type = other DNA
tcgctgttgccaaacgcgtc

SEQ ID NO: 253 - DNA, 20 bp, synthetic construct
  source 1..20: mol_type = other DNA
gttttattttactaaagagg

SEQ ID NO: 254 - DNA, 18 bp, synthetic construct
  source 1..18: mol_type = other DNA
gtatagaacagcaaatct

SEQ ID NO: 255 - DNA, 18 bp, synthetic construct
  source 1..18: mol_type = other DNA
ggcagaaacttttacttt

SEQ ID NO: 256 - DNA, 18 bp, synthetic construct
  source 1..18: mol_type = other DNA
atttgaaataaacttacc

SEQ ID NO: 257 - DNA, 18 bp, synthetic construct
  source 1..18: mol_type = other DNA
attaccagccatcttatt

SEQ ID NO: 258 - DNA, 18 bp, synthetic construct
  source 1..18: mol_type = other DNA
attgtatagaacagcaaa

SEQ ID NO: 259 - DNA, 18 bp, synthetic construct
  source 1..18: mol_type = other DNA
ttgttcaagcaacataca

SEQ ID NO: 260 - DNA, 18 bp, synthetic construct
  source 1..18: mol_type = other DNA
gcttgtgtcaacaaaaca

SEQ ID NO: 261 - DNA, 20 bp, synthetic construct
  source 1..20: mol_type = other DNA
gcaatcctgtagatttgagt

SEQ ID NO: 262 - DNA, 20 bp, synthetic construct
  source 1..20: mol_type = other DNA
gctaggattgccgcgaccgg

SEQ ID NO: 263 - DNA, 18 bp, synthetic construct
  source 1..18: mol_type = other DNA
catataactcatcaatgt

SEQ ID NO: 264 - DNA, 20 bp, synthetic construct
  source 1..20: mol_type = other DNA
actctttcaacctgggatca

SEQ ID NO: 265 - DNA, 18 bp, synthetic construct
  source 1..18: mol_type = other DNA
aaagttaaggcttagagt

SEQ ID NO: 266 - DNA, 20 bp, synthetic construct
  source 1..20: mol_type = other DNA
ttcattaccagccatcttat

SEQ ID NO: 267 - DNA, 18 bp, synthetic construct
  source 1..18: mol_type = other DNA
tgtaataaatttatgaac

SEQ ID NO: 268 - DNA, 18 bp, synthetic construct
  source 1..18: mol_type = other DNA
tagcactttcatataact

SEQ ID NO: 269 - DNA, 20 bp, synthetic construct
  source 1..20: mol_type = other DNA
aacaattgctccagcattta

SEQ ID NO: 270 - DNA, 20 bp, synthetic construct
  source 1..20: mol_type = other DNA
ctgaggccaccagctctttg

SEQ ID NO: 271 - DNA, 20 bp, synthetic construct
  source 1..20: mol_type = other DNA
agctaggtaacagaccctgt

SEQ ID NO: 272 - DNA, 20 bp, synthetic construct
  source 1..20: mol_type = other DNA
gtatagaacagcaaatcttc

SEQ ID NO: 273 - DNA, 20 bp, synthetic construct
  source 1..20: mol_type = other DNA
gcaattgtatagaacagcaa

SEQ ID NO: 274 - DNA, 20 bp, synthetic construct
  source 1..20: mol_type = other DNA
tctagcatgacaccatctga

SEQ ID NO: 275 - DNA, 20 bp, synthetic construct
  source 1..20: mol_type = other DNA
agatctttgtctagcatgac

SEQ ID NO: 276 - DNA, 20 bp, synthetic construct
  source 1..20: mol_type = other DNA
gcttgtgtcaacaaaacaat

SEQ ID NO: 277 - DNA, 20 bp, synthetic construct
  source 1..20: mol_type = other DNA
atgtgtgaggtaaaagacat

SEQ ID NO: 278 - DNA, 20 bp, synthetic construct
  source 1..20: mol_type = other DNA
ggccagttgaggaatataat

SEQ ID NO: 279 - DNA, 20 bp, synthetic construct
  source 1..20: mol_type = other DNA
tgaatttggccagttgagga

SEQ ID NO: 280 - DNA, 20 bp, synthetic construct
  source 1..20: mol_type = other DNA
actgtacaaacagacacacc

SEQ ID NO: 281 - DNA, 20 bp, synthetic construct
  source 1..20: mol_type = other DNA
ccatctactgtacaaacaga

SEQ ID NO: 282 - DNA, 20 bp, synthetic construct
  source 1..20: mol_type = other DNA
tttacacaggactgaagaca

SEQ ID NO: 283 - DNA, 20 bp, synthetic construct
  source 1..20: mol_type = other DNA
tcaaaggttttacacaggac

SEQ ID NO: 284 - DNA, 20 bp, synthetic construct
  source 1..20: mol_type = other DNA
cattaacagcaatggcatat

SEQ ID NO: 285 - DNA, 20 bp, synthetic construct
  source 1..20: mol_type = other DNA
ccagcatttaccataggatt

SEQ ID NO: 286 - DNA, 20 bp, synthetic construct
  source 1..20: mol_type = other DNA
tcagactgaaacctggaaga

SEQ ID NO: 287 - DNA, 20 bp, synthetic construct
  source 1..20: mol_type = other DNA
ccactttctctttcagactg

SEQ ID NO: 288 - DNA, 20 bp, synthetic construct
  source 1..20: mol_type = other DNA
tgtgccttctggaaaacact

SEQ ID NO: 289 - DNA, 20 bp, synthetic construct
  source 1..20: mol_type = other DNA
acctggaagtagaagtctaa

SEQ ID NO: 290 - DNA, 20 bp, synthetic construct
  source 1..20: mol_type = other DNA
tcacttcaatggagcacagc

SEQ ID NO: 291 - DNA, 20 bp, synthetic construct
  source 1..20: mol_type = other DNA
ccatcacactggctgattca

SEQ ID NO: 292 - DNA, 20 bp, synthetic construct
  source 1..20: mol_type = other DNA
ccagtaattgggcagaaacc

SEQ ID NO: 293 - DNA, 20 bp, synthetic construct
  source 1..20: mol_type = other DNA
gaccagcacatcatacccat

SEQ ID NO: 294 - DNA, 20 bp, synthetic construct
  source 1..20: mol_type = other DNA
gaattcccttaacactgttg

SEQ ID NO: 295 - DNA, 20 bp, synthetic construct
  source 1..20: mol_type = other DNA
ttacacagagaaacaagatc

SEQ ID NO: 296 - DNA, 20 bp, synthetic construct
  source 1..20: mol_type = other DNA
gcaaagtgtctcaaattatc

SEQ ID NO: 297 - DNA, 20 bp, synthetic construct
  source 1..20: mol_type = other DNA
cctttgatcaccaccttctc

SEQ ID NO: 298 - DNA, 20 bp, synthetic construct
  source 1..20: mol_type = other DNA
tcaacctgggatcagacgtt

SEQ ID NO: 299 - DNA, 20 bp, synthetic construct
  source 1..20: mol_type = other DNA
agaacagcaaatcttccaag

SEQ ID NO: 300 - DNA, 18 bp, synthetic construct
  source 1..18: mol_type = other DNA
cactctttcaacctggga

SEQ ID NO: 301 - DNA, 18 bp, synthetic construct
  source 1..18: mol_type = other DNA
atagaacagcaaatcttc

SEQ ID NO: 302 - DNA, 18 bp, synthetic construct
  source 1..18: mol_type = other DNA
gcaattgtatagaacagc

SEQ ID NO: 303 - DNA, 18 bp, synthetic construct
  source 1..18: mol_type = other DNA
gtatagaacagcaaatct

SEQ ID NO: 304 - DNA, 4840 bp, Homo sapiens
  source 1..4840: mol_type = genomic DNA
agtgcggagccttaggcggagcgaagagaaccggtcgcggcaatcctagcgcgcagcagcagcagcagcagcagcagcagcagcagcagcagcagcagcacccgcatccgctgcgggagtccgagccggaaccacacccaagtagctgccctttcctcttctgtcatctcaccgccccaccacagaccgcgttccccgaggaaaccggccgcccacgcccggagcatcctcccctgttgagcgggcgctgacggacccggcggcatgatgcggctgcgaggctcggggatgctgcgggacctgctcctgcggtcgcccgccggcgtgagcgcgactctgcggcgggcacagcccttggtcaccctgtgccggcgtccccgaggcgggggacggccggccgcgggcccggctgccgccgcgcgactccacccgtggtggggcgggggcggctggccggcggagcccctcgcgcggggcctgtccagctctccttcggagatcttgcaggagctgggcaaggggagcacgcatccgcagcccggggtgtcgccacccgctgccccggcggcgcccggccccaaggacggccccggggagacggacgcgtttggcaacagcgagggcaaagagctggtggcctcaggtgaaaataaaataaaacagggtctgttacctagcttggaagatttgctgttctatacaattgctgaaggacaagagaaaatacctgttcataaatttattacagcactcaaatctacaggattgcgaacgtctgatcccaggttgaaagagtgtatggatatgttaagattaactcttcaaacaacatcagatggtgtcatgctagacaaagatctttttaaaaaatgtgttcagagcaacattgttttgttgacacaagcatttagaagaaagtttgtgattcctgactttatgtcttttacctcacacattgatgagttatatgaaagtgctaaaaagcagtctggaggaaaggttgcagattatattcctcaactggccaaattcagtcccgatttgtggggtgtgtctgtttgtacagtagatggacagaggcattctactggagataccaaagttcccttctgtcttcagtcctgtgtaaaacctttgaaatatgccattgctgttaatgatcttggaactgaatatgtgcatcgatatgttggaaaagagccgagtggactaagattcaacaaactatttttgaatgaagatgataaaccacataatcctatggtaaatgctggagcaattgttgtgacttcactaataaagcaaggagtaaataatgctgaaaaatttgactatgtcatgcagtttttgaataagatggctggtaatgaatatgttggattcagtaatgcaacgtttcagtctgaaagagaaagtggagatcgaaattttgcaataggatattacttaaaagaaaagaagtgttttccagaaggcacagacatggttggtatattagacttctacttccagctgtgctccattgaagtgacttgtgaatcagccagtgtgatggctgcgacactggctaatggtggtttctgcccaattactggtgaaagagtactgagccctgaagcagttcgaaatacattgagtttgatgcattcctgtggcatgtatgacttctcagggcagtttgctttccatgttggtcttcctgcaaaatctggagttgctgggggcattcttttagttgtccccaatgttatgggtatgatgtgctggtctcctcctctggataagatgggcaacagtgttaagggaattcacttttgtcacgatcttgtttctctgtgtaatttccataactatgataatttgagacactttgcaaaaaaacttgatcctcgaagagaaggtggtgatcaaagggtaaagtcagtgataaatcttttgtttgctgcatatactggagatgtgtctgcacttcgaagatttgctttgtcagctatggacatggaacagcgggactatgattctagaacagcactccatgtagctgctgcagagggtcatgttgaagttgttaaatttttgctggaagcctgcaaagtaaaccctttccccaaggacaggtggaataacactcccatggatgaagcactgcactttggacaccatgatgtatttaaaattctccaagaataccaagtccagtacacacctcaaggagattctgacaacgggaaggaaaatcaaaccgtccataagaatcttgatggattgttgtaatggtctcaaatcccaagatttaaatcacttacctatttaattgtggaaaatgattatgaagaacatgtgtatttctatctggtagtgatgtatattttacatttgtcatttcagtgttactggagttttcttcattgtgcacacaggacaaatctgatctctttgggaaaaaatagaaataaaacaatctccctccataatgtgagcaatattacctcgtgcattgtataatttgatgtaaaagaaatagttaccaatgctagcttgtgtggtcttccatgatttatttgtgttttgtgaattttcaatttatggtgatgatctgctgatatgcatttataaagtaagctctgttgtacagtctgtccaaatgggtcaaggttgcctttagaagcaaatagtgtgattttcaagacttcaaatacaaatttagtttaagtgtttgaacaactatatgcacttacggttgtgtgtttaaaatgtctctctcaccccctagcttcatgatgtgactcttaaaaaactataatagttaacaactgttagtaagatagaccaattctgattagactttatcagggaatctgtttaagatatgtttggtgaccaaaacgtatgtgtgaatgtagttataatgcttttgaaaaattttcctttttctatatccccttagtccagcctctcttctcagacatttagctatctgcctctttcctttagctgggaaagtgagagctggcatactatgcagtttttatgttttccatagtaagtcagaaaatgcctcctatttctggcatcagaactttgccatttgtctacagaagacgaaccagagacaaaattactaagtataaattagtcaagtttatcagtctaaaaaacgaagggatgtgcaactgcagctctttaagaagttttttttttttagcttctagggtaaagataaattcagaaatgctctaagctaccaaagttattctgaaagtatgggaactgctacaactaacaaacatttgtttccaagcctgtcattaagagtctgcatcaagagatttgtcctccttgggggaccactggatcattccagatttcttgtgatttttctattgtgtaattcttggtgggctctgtagtttaataataagaaaaaggccatttcattttaaattgtgacctataattctttgtcttgggttggtaattcaggattcatttggaaagtgggtaaaaggggcttcaaaaaacggatagaacaggattttctaggagttacacatacattttatcctgtcatacctcgagataaagtggcatgttagtgaggagttctgatattaagcacacacacacatgcacacaaatggacttctctgaagctgtgtttagtgaaatgagctcaagtacatgaatgttagttgttatcacatacagcaaattcctttttttttctttttctatgagcacactctgctgcttctaaactttacatgcctgatggcaccttactccagcagcctccaggtgctttcattttcacttccagtctaagccagtggctcctgccactgccctcccattacctagatggcacctcctttggtgaaaccacggccaatgttccttagctgcaccaggcccgaagctgttcccatgcttgagcttccatggggaggatgctgagtgagcagtttcctaccccgtggatctagcaagccatggagacaggtagcatttgtaagatgctgcacaggagcagcattatccccaaagatattacagggtagacacgttttaactgaaatcaatcaagataactttattcaaagagcagcccgctttgtgtgactaaaatgaaacaagacagttgaattgtgtgacttgaagattaccaatgattttgaggcttttctataataaaaagaggttctaaccattatttgggaacaaagagagttttcatcttttttcagatcaaaaccattctgtaaaatctttgttgtttaattaaatgtgccgttatttacccctgatgttatttatgactatgtgccgattcctgctcgggctgtttgctgttggctggtaataatatatttgatttaaatgctgttgactgtgctattaactgctgccgtcagtaaactccaaagatctttttgttttggctttagtatcatatgtgctttttctgtatcctgagcgctctatatgatcatgttaatttaaagctttatacacattgttgtttttgctggtctcatctttggtaatatgctataccccactgctgcccgacactgccctttagctgcagagctggattagctgttgaccatttgatgctgttgtctgtctggcagggactgaatgacctgatgtcagatttagattcttcctggggattacacagctatgaatgtatttgcttctaaaacctcccaaagtgaatctaatcttaaaactacaagttgtaagtattctgaaattgggaaacatttattttaaatgcaatcaggtagtgttgctttttacagcataataaatatatgtatcaaaaaaaaaa

SEQ ID NO: 305 - DNA, 1000 bp, Homo sapiens
  source 1..1000: mol_type = genomic DNA
agtgcggagccttaggcggagcgaagagaaccggtcgcggcaatcctagcgcgcagcagcagcagcagcagcagcagcagcagcagcagcagcagcagcacccgcatccgctgcgggagtccgagccggaaccacacccaagtagctgccctttcctcttctgtcatctcaccgccccaccacagaccgcgttccccgaggaaaccggccgcccacgcccggagcatcctcccctgttgagcgggcgctgacggacccggcggcatgatgcggctgcgaggctcggggatgctgcgggacctgctcctgcggtcgcccgccggcgtgagcgcgactctgcggcgggcacagcccttggtcaccctgtgccggcgtccccgaggcgggggacggccggccgcgggcccggctgccgccgcgcgactccacccgtggtggggcgggggcggctggccggcggagcccctcgcgcggggcctgtccagctctccttcggagatcttgcaggagctgggcaaggggagcacgcatccgcagcccggggtgtcgccacccgctgccccggcggcgcccggccccaaggacggccccggggagacggacgcgtttggcaacagcgagggcaaagagctggtggcctcaggtgaaaataaaataaaacagggtctgttacctagcttggaagatttgctgttctatacaattgctgaaggacaagagaaaatacctgttcataaatttattacagcactcaaatctacaggattgcgaacgtctgatcccaggttgaaagagtgtatggatatgttaagattaactcttcaaacaacatcagatggtgtcatgctagacaaagatctttttaaaaaatgtgttcagagcaacattgttttgttgacacaagcatttagaagaaagtttgtgattcctgactttatgtcttttacctcacacattgatgagttatatgaaagtgctaaaaagcagtctggaggaaagg

(19) United States
(12) Patent Application Publication
(10) Pub. No.: US 2026/0275349 A1
(43) Pub. Date: Sep. 17, 2026

(54) COMPOSITIONS AND METHODS FOR MODULATING GLS1

(71) Applicant: Leal Therapeutics, Inc., Worcester, MA (US)

(72) Inventors: Asa Abeliovich, New York, NY (US); Laura Heckman, Philadelphia, PA (US); Herve Rhinn, Paris (FR); Vinod Vathipadiekal, Stoneham, MA (US)

(73) Assignee: Leal Therapeutics, Inc., Worcester, MA (US)

(21) Appl. No.: 19/163,133

(22) PCT Filed: Mar. 8, 2024
(86) PCT No.: PCT/US2024/019067

§ 371 Date: Sep. 8, 2025

Related U.S. Application Data
(60) Provisional application No. 63/489,237, filed on Mar. 9, 2023.
(60) Provisional application No. 63/492,589, filed on Mar. 28, 2023.
(60) Provisional application No. 63/507,987, filed on Jun. 13, 2023.
(60) Provisional application No. 63/580,784, filed on Sep. 6, 2023.
(60) Provisional application No. 63/588,178, filed on Oct. 5, 2023.
(60) Provisional application No. 63/556,389, filed on Feb. 21, 2024.

(51) Int. Cl. C12N 15/113; A61K 31/712; A61K 31/7125; A61P 25/18

(52) U.S. Cl. CPC C12N 15/1137; A61K 31/712; A61K 31/7125; A61P 25/18; C12N 2310/11; C12N 2310/315; C12N 2310/321; C12N 2310/3231; C12N 2310/341; C12N 2310/346; C12N 2320/33

(57) ABSTRACT

Aspects of the disclosure relate to compositions and methods for modulating levels, transcription, splicing, and/or translation of one or more RNA transcripts (e.g., mRNA transcripts) in a cell or subject. The disclosure is based, in part, on isolated nucleic acids that bind to mRNA transcripts of genes involved in psychiatric diseases and disorders, for example glutaminase (GLS1\ which is the enzyme responsible for producing glutamate in the central nervous system (CNS). In some embodiments, compositions of the disclosure are useful for treating psychiatric diseases or disorders, such as schizophrenia (e.g., treatment resistant schizophrenia), Alzheimer's disease (AD), Parkinson's disease (PD), or neuroinflammation.

### RELATED APPLICATIONS

This application is a national stage filing under 35 U.S.C. § 371 of international PCT application PCT/US2024/019067, filed Mar. 8, 2024, which claims priority under 35 U.S.C. § 119(e) to U.S. Provisional Application No. 63/489,237 filed on Mar. 9, 2023, U.S. Provisional Application No. 63/492,589 filed on Mar. 28, 2023, U.S. Provisional Application No. 63/507,987 filed on Jun. 13, 2023, U.S. Provisional Application No. 63/580,784 filed on Sep. 6, 2023, U.S. Provisional Application No. 63/588,178 filed on Oct. 5, 2023, and U.S. Provisional Application No. 63/556,389 filed on Feb. 21, 2024, each of which is herein incorporated by reference in its entirety.

### REFERENCE TO AN ELECTRONIC SEQUENCE LISTING

The contents of the electronic sequence listing (L090770039US06-SEQ-KZM.xml; Size: 275,108 bytes; and Date of Creation: Sep. 5, 2025) is herein incorporated by reference in its entirety.

### BACKGROUND

Glutamate is the anion of glutamic acid and acts as an excitatory neurotransmitter that is involved in modulating a variety of biological processes. In the central nervous system (CNS), glutamate is produced from glutamine by the enzyme glutaminase. Glutamate signals through three different receptor types: AMPA receptors, NMDA receptors, and metabotropic glutamate receptors. Glutamate signaling through NMDA receptors is important for controlling synaptic plasticity and mediating learning and memory functions.

### SUMMARY

Aspects of the disclosure relate to isolated nucleic acids that bind to mRNA transcripts of genes involved in certain psychiatric diseases and disorders, for example a gene encoding glutaminase (GLS1). In some embodiments, compositions of the disclosure are useful for treating diseases or disorders associated with psychiatric diseases and disorders, such as schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.). In some embodiments, compositions of the disclosure are useful for treating neuroinflammation. The disclosure is based, in part, on compositions and methods for modulating a level, transcription, splicing, and/or translation of one or more RNA transcripts (e.g., mRNA transcripts) in a cell or subject.

Accordingly, in some aspects, the disclosure provides an isolated nucleic acid that comprises a region of complementarity with a human GLS1 mRNA transcript, has at least 60% identity (e.g., 60-70%, 70-80%, 80-90%, 90-95%, 95-99%, or 100% identity) to a nucleic acid the nucleic acid sequence set forth in any one of SEQ ID NOs: 1-303, and upon binding to the mRNA transcript decreases a level, transcription, splicing, and/or translation of functional glutaminase protein from the mRNA transcript.

In some embodiments, the isolated nucleic acid comprises RNA. In some embodiments, the isolated nucleic acid is an antisense oligonucleotide (ASO).

In some embodiments, the isolated nucleic acid comprises or consists of between 10 and 40 nucleotides. In some embodiments, the isolated nucleic acid comprises or consists of between 18 and 25 nucleotides.

In some embodiments, the isolated nucleic acid comprises one or more chemical modifications. In some embodiments, the one or more chemical modifications comprise one or more nucleoside modifications and/or one or more sugar-phosphate backbone modifications. In some embodiments, the one or more nucleoside modifications comprises a 2′-O-methyl (2′-OMe) modification, 2′-fluoro modification, or a locked nucleic acid (LNA) modification. In some embodiments, the one or more sugar-phosphate backbone modifications comprises a phosphorothioate backbone modification. In some embodiments, the isolated nucleic acid is fully chemically modified (e.g., contains a fully modified sugar-phosphate backbone, and all nucleotides of the isolated nucleic acid are chemically modified).

In some embodiments, the isolated nucleic acid comprises one or more deoxyribonucleotides. In some embodiments, the isolated nucleic acid is a gapmer.

In some embodiments, the region of complementarity is located in an untranslated region of the GLS1 mRNA transcript. In some embodiments, the untranslated region comprises a 5′UTR, intron, or 3′UTR of the GLS1 mRNA transcript.

In some embodiments, the region of complementarity is located in a protein coding region of the GLS1 mRNA transcript.

In some embodiments, the region of complementarity is located on an intron-exon boundary (e.g., the region of complementarity spans an intron exon boundary, such that the isolated nucleic acid hybridizes binds to both an intron and an exon at the same time) of the GLS1 mRNA transcript.

In some embodiments, the region of complementarity comprises at least 10, 11, 12, 13, 14, 15, 16, 17, 18, 19, or 20 continuous nucleotides of the sequence set forth in SEQ ID NO: 304.

In some embodiments, the nucleotide sequence comprises the nucleic acid sequence set forth in any one of the nucleotide sequences set forth in Table 1.

In some aspects, the disclosure provides a method for decreasing glutamate signaling in a cell or subject, the method comprising administering an isolated nucleic acid as described herein to a subject in need thereof.

In some embodiments, the cell is a neuronal cell. In some embodiments, the neuronal cell is a presynaptic neuronal cell.

In some embodiments, the subject comprises one or more mutations in a gene that is associated with glutamate signaling. In some embodiments, the gene is GLS1.

In some embodiments, the cell or subject is a human cell or subject.

In some embodiments, the subject has or is suspected of having a psychiatric disease or disorder. In some embodiments, the disease or disorder is schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.). In some embodiments, the subject has neuroinflammation.

In some embodiments, the administration is systemic administration. In some embodiments, the systemic administration comprises intravenous injection.

In some embodiments, the administration comprises direct administration to a target tissue of the subject. In some embodiments, the direct administration comprises direct injection to the central nervous system (CNS) of the subject. In some embodiments, the direct administration comprises direct injection to the peripheral nervous system (PNS) of the subject.

In some embodiments, the administration comprises placing the subject in a Trendelenburg position during the administration.

In some aspects, the disclosure provides a method for reducing neuroinflammation in a subject, the method comprising administering an isolated nucleic acid as described herein, to a subject in need thereof.

In some embodiments, the subject does not comprise one or more mutations in a gene associated with glutamate signaling. In some embodiments, a subject does not have a mutation in a GLS1 gene.

In some embodiments, the subject comprises one or more mutations in a gene that is associated with glutamate signaling. In some embodiments, the gene is GLS1.

In some embodiments, the subject is a human subject.

In some embodiments, the subject has or is suspected of having a psychiatric disease or disorder. In some embodiments, the psychiatric disease or disorder is schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.).

In some embodiments, the administration is systemic administration. In some embodiments, the systemic administration comprises intravenous injection.

In some embodiments, the administration comprises direct administration to a target tissue of the subject. In some embodiments, the direct administration comprises direct injection to the central nervous system (CNS) of the subject. In some embodiments, the direct administration comprises direct injection to the peripheral nervous system (PNS) of the subject.

In some embodiments, the administration comprises placing the subject in a Trendelenburg position during the administration.

In some aspects, the disclosure provides a method for preventing or treating a psychiatric disease or disorder in a subject in need thereof, the method comprising administering to the subject an isolated nucleic acid as described herein.

In some embodiments, the subject is a human.

In some embodiments, the psychiatric disease or disorder is schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.).

In some embodiments, the administration comprises direct administration to a target tissue of the subject. In some embodiments, the direct administration comprises direct injection to the central nervous system (CNS) of the subject. In some embodiments, the direct administration comprises direct injection to the peripheral nervous system (PNS) of the subject.

### BRIEF DESCRIPTION OF DRAWINGS

FIG. 1 shows a schematic depicting modulation of RNA (e.g., mRNA, such as mature mRNA or pre-mRNA) translation by antisense oligonucleotides (ASOs). Composition “A” represents an ASO that binds to the 5′ untranslated region (5′ UTR) of an RNA. Composition “B” represents an ASO that binds to an intron of an RNA. Composition “C” represents an ASO that binds to a splice boundary (e.g., a splice junction) between an exon and intron of an RNA. Composition “D” represents an ASO that binds to an exon (e.g., protein coding region) of an RNA. Composition “E” represents a combination of an ASO binding to a 3′ UTR of an RNA, alone or with a trans-regulator. Composition “F” represents a “gapmer” ASO that binds to an exon (e.g., protein coding region) of an RNA and mediates RNaseH decay. Composition “G” represents a “gapmer” ASO that binds to a 3′ UTR of an RNA, alone or with a trans-regulator, and mediates RNaseH decay. In some embodiments, ASOs binding to an RNA result in translation of a truncated protein that has a dominant negative effect on the wild-type, full-length protein.

FIGS. 2A-2C show representative data regarding expression profiling of human glutaminase (GLS1). FIG. 2A shows bulk tissue gene expression of human GLS1; data indicate GLS1 mRNA is expressed in various tissues. FIG. 2B shows a schematic depicting exons and introns present in the GLS1 gene. FIG. 2C shows representative data for exon expression analysis of human GLS1 splice variants in tissue.

FIG. 3 is a schematic depicting the primary AUG, and several exon-exon junctions of GLS1 mRNA transcript (SEQ ID NO: 305).

FIGS. 4A-4C show representative in vitro data for ASOs targeting GLS1 RNA. FIG. 4A shows a diagram of GLS1 RNA within which exons are indicated as boxes and introns are indicated as dashed lines. The 192 designed ASOs (grey and dark grey rectangles below GLS1 mRNA diagram; Table 1) are shown based on the location of their target region on GLS1 RNA. Sixteen of the most potent ASOs of each chemistry, resulting in at least 50% reduction in GLS1 RNA at a 5 nM dose, are indicated by dark and light shading. FIG. 4B shows GLS1 RNA levels after ASO treatment. Normalized levels of GLS1 RNA in U-251 MG cells as measured by branched DNA (bDNA) in a bDNA signal amplification assay 48 hours after transfection with mock and non-targeting control ASOs (grey) or ASOs targeting GLS1 at both 5 nM and 20 nM doses (chemistry 1: light and dark shaded bars to the right of controls, respectively; chemistry 2: light and dark shaded bars at the far right, respectively) are presented. GLS1 expression was normalized to GAPDH expression and presented as a percentage of the negative mock and non-targeting controls. Means across biological replicates (N=4) are presented; error bars are standard error of the mean. FIG. 4C shows GLS1 RNA down-regulation by select ASOs. Normalized levels of GLS1 RNA in U-251 MG cells, as measured by bDNA 48 hours after transfection with mock and non-targeting control ASOs (grey) or 16 of the most potent GLS1 ASOs of each chemistry at both 5 nM and 20 nM doses (chemistry 1: light and dark shaded bars to right of controls, respectively; chemistry 2: rightmost set of two 1 light and dark shaded bars, respectively) are presented. GLS1 expression was normalized to GAPDH expression and presented as a percentage of the negative mock and non-targeting mock controls. Means across biological replicates (N=4) are presented; error bars are standard error of the mean.

FIG. 5 shows high concordance of GLS1 knockdown in cells treated during separate two-dose and ten-dose series. 32 ASOs targeting GLS1 were tested in U-251 MG cells as part of a ten-dose series and knockdown was compared with that observed previously in U-251 MG cells treated in a two-dose series. U-251 MG cells were reverse transfected with ASOs at either a 5 nM dose or a 20 nM dose (chemistry 1 (skipmer, alternatively referred to as an “exon-skipper” or “skipper” chemistry herein): light and dark shaded circles and triangles, respectively; chemistry 2 (gapmer): light and dark shaded circles and triangles, respectively) and GLS1 expression was measured after 48 hours by RT-qPCR. The effect of tested ASOs on cells treated as part of either series was determined to be highly correlated (R=0.88).

FIG. 6 shows dose response of GLS1-targeting ASOs in U-251 MG cells. Seven distinct ASOs were assayed in U-251 MG cells in vitro at 10 doses: 40 nM, 20 nM, 10 nM, 5 nM, 2.5 nM, 1.25 nM, 0.625 nM, 0.3125 nM, 0.15625 nM, and 0.078125 nM. U-251 MG cells were treated in 96 well plate format by forward transfection and GLS1 expression was assayed after 48 hours by bDNA assay. ASO treatments were normalized to control transfected cells. 0% knockdown and 50% knockdown are indicated by dashed lines (black and grey, respectively). Means across biological replicates (N=4) are presented; error bars are standard deviation.

FIG. 7 shows representative data for in vitro dose-response reduction of GLS1 mRNA in U251-MG glioma cells. The top panel shows GLS1 mRNA levels presented relative to mock-transfected controls in function of the dose of transfected ASOs for the 15 tested GLS1 ASOs (Skippers (alternatively referred to as an “exon-skipper” or “skipmer” chemistry herein) and Gapmers shown). Means are presented, error bars are Standard Error for N=2 biological replicates by group. The bottom panel shows maximum inhibition (log 2, Y-axis) plotted in function of the observed EC50 (X-axis); the most potent ASOs are in the lower left part of the dot-plot.

FIGS. 8A-8E show representative data for in vivo reduction of GLS1 mRNA levels in mouse brain. FIG. 8A shows relative GLS1 mRNA levels 3 weeks after a single bilateral stereotaxic ICV injection of vehicle (artificial CSF) or 100 ug of a GLS1 ASO comprising the nucleotide sequence of SEQ ID NO: 34 as set forth in Column A of row 35 in Table 1 and a skipmer chemistry (alternatively referred to as an “exon-skipper” or “skipper” chemistry herein) according to the chemical modifications as set forth in Column C of row 35 in Table 1. FIG. 8B shows relative GLS1 mRNA levels one week after the last of three ICV injections through a canula, with 1 week interval, of vehicle (artificial CSF), 100 ug+100 ug+100 ug or 200 ug+200 ug+100 ug of a GLS1 ASO comprising the nucleotide sequence of SEQ ID NO: 34 as set forth in Column A of row 35 in Table 1 and a skipmer chemistry (alternatively referred to as an “exon-skipper” or “skipper” chemistry herein) according to the chemical modifications as set forth in Column C of row 35 in Table 1. FIG. 8C shows relative GLS1 mRNA levels in hippocampus tissues of mouse subjects 7 days after a series of three weekly ICV injections of vehicle (artificial CSF), 3 ug of myriocin, a non-specific ASO at a dose of 200 ug (100 ug+50 ug+50 ug) or 300 ug (100 ug+100 ug+100 ug), or GLS1 ASO comprising the nucleotide sequence of SEQ ID NO: 233, a gapmer structure, and the chemical modifications as set forth in Columns A and C of row 234 of Table 1 at a dose of 300 ug (100 ug+100 ug+100 ug). FIG. 8D shows relative GLS1 mRNA levels in cortex tissues of mouse subjects seven days after a series of three weekly ICV injections of vehicle (artificial CSF), 3 ug of myriocin, a non-specific ASO at a dose of 200 ug (100 ug+50 ug+50 ug) or 300 ug (100 ug+100 ug+100 ug), or GLS1 ASO comprising the nucleotide sequence of SEQ ID NO: 233, a gapmer structure, and the chemical modifications as set forth in Columns A and C of row 234 of Table 1 at a dose of 300 ug (100 ug+100 ug+100 ug). FIG. 8E shows relative GLS1 mRNA levels one week after the last of three ICV injections through a canula, with 1 week interval, of vehicle (artificial CSF) or 100 ug+100 ug+100 ug of a GLS1 ASO comprising the nucleotide sequence of SEQ ID NO: 233, a gapmer structure, and the chemical modifications as set forth in Columns A and C of row 234 of Table 1.

FIGS. 9A-9K show representative data for GLS1 ASO pharmacokinetics (ASO levels) and pharmacodynamics (GLS1 mRNA expression) in frontal cortex (“Cortex”), hippocampus, and lumbar spinal (“Lumbar SC”) cord tissue samples obtained from injected non-human primate subjects. “GLS1 ASO” comprises the nucleotide sequence of SEQ ID NO: 34 as set forth in Column A of row 35 in Table 1 and a skipmer chemistry (alternatively referred to as an “exon-skipper” or “skipper” chemistry herein) according to the chemical modifications as set forth in Column C of row 35 in Table 1. N=2-3 and each dot on the graph represents a respective non-human primate subject. FIG. 9A shows a non-limiting example of a study design wherein non-human primate subjects were administered a series of two intrathecal (IT) injections performed on days 1 and 14 of either vehicle (artificial CSF) or GLS1 ASO at a dose of 40 mg (20 mg+20 mg). FIG. 9B shows GLS1 ASO levels in frontal cortex, hippocampus, and lumbar spinal cord tissue samples obtained from injected non-human primate subjects that underwent administration according to FIG. 9A. “pg/g” refers to micrograms of GLS1 ASO per gram of tissue sample. Bars indicate mean+/−standard deviation. FIG. 9C shows RT-qPCR analysis data which was obtained using two different GLS1 probes (dark and light shaded bars) measuring GLS1 mRNA expression in frontal cortex tissue of non-human primate subjects harvested after intrathecal injections with either vehicle (artificial CSF), a non-specific ASO at a dose of 20 mg (10 mg+10 mg) or 40 mg (20 mg+20 mg), or GLS1 ASO at a dose of 40 mg (20 mg+20 mg) according to FIG. 9A. GLS1 mRNA expression was normalized to PGK1 mRNA expression and shown as percent of vehicle group. Bars indicate mean+/−standard deviation. FIG. 9D shows a comparison of the data in FIG. 9C obtained by RT-qPCR analysis of GLS1 mRNA expression in frontal cortex tissue of non-human primate subjects that underwent administration according to FIG. 9A. GLS1 mRNA expression was normalized to PGK1 mRNA expression and shown as percent of vehicle group. *:p<0.05 for treatment vs. vehicle by unpaired T-test. Bars indicate mean+/−standard deviation. FIG. 9E shows RT-qPCR analysis data which was obtained using two different GLS1 probes (dark and light shaded bars) measuring GLS1 mRNA expression in frontal cortex tissue of non-human primate subjects harvested after intrathecal injections with either vehicle (artificial CSF), a non-specific ASO, or a GLS1 ASO. GLS1 mRNA expression was normalized to PGK1 mRNA expression and shown as percent of vehicle group. *p<0.05 for treatment vs. vehicle by unpaired T-test. Bars indicate mean+/−standard deviation. FIG. 9F shows RT-qPCR analysis data for GLS1 mRNA expression in frontal cortex tissue samples obtained from injected non-human primate subjects that that received GLS1 ASO. *p<0.05 for treatment vs. vehicle by unpaired T-test. Bars indicate mean+/−standard error of the mean. FIG. 9G shows RT-qPCR analysis data which was obtained using two different GLS1 probes (dark and light shaded bars) measuring GLS1 mRNA expression in sensory cortex tissue of non-human primate subjects harvested after intrathecal injections with either vehicle (artificial CSF), a non-specific ASO, or a GLS1 ASO. GLS1 mRNA expression was normalized to PGK1 mRNA expression and shown as percent of vehicle group. (*) p<0.1 for treatment vs. vehicle by unpaired T-test. Bars indicate mean+/−standard deviation. FIG. 9H shows RT-qPCR analysis data for GLS1 mRNA expression in sensory cortex tissue samples obtained from injected non-human primate subjects that that received GLS1 ASO. (*) p<0.1 for treatment vs. vehicle by unpaired T-test. Bars indicate mean+/−standard error of the mean. FIG. 9I shows RT-qPCR analysis data which was obtained using two different GLS1 probes (left and right panels) measuring GLS1 mRNA expression in sensory cortex tissue of non-human primate subjects harvested after intrathecal injections with either vehicle (artificial CSF) or a high dose of ASO. GLS1 mRNA expression was normalized to PGK1 mRNA expression and shown as percent of vehicle group. Bars indicate mean+/−standard deviation. (*) p<0.1 for treatment vs. vehicle by unpaired T-test. FIG. 9J shows a comparison of pharmacodynamics in frontal cortex (left panel) and sensory cortex (right panel) tissue samples as a result of intrathecal administration of GLS1 ASO (*) p<0.1; *p<0.05 for treatment vs. vehicle by unpaired T-test. Bars indicate mean+/−standard error of the mean. FIG. 9K shows a table representation of the data shown in FIGS. 9C-9J. Mean knockdown levels are shown as the percent difference from the vehicle group. Mean knockdown levels were analyzed over a 95% confidence interval (“CI”) (lower and upper limits shown in brackets). P-values associated with the mean knockdown levels as determined from confidence interval analysis are shown in scientific notation. The “IDT” probe and the “Thermo” probe corresponds to the dark and light shaded bars, respectively, in FIGS. 9C-9H and 9J.

FIGS. 10A-10J show representative immunostimulatory effects of GLS1 ASO on human peripheral blood mononuclear cells (huPBMCs) that were harvested from healthy donors. huPBMCs were either untreated (“mock” and “media”), treated with a cytokine/chemokine response control agent (XD-01024, XD00366 transfection, poly(l:c) transfection, CL097, R837, TL8-506, ODN2395 transfection, ODN2395 gymnotic, ODN2216 transfection, ODN2216 gymnotic, ODN2006 transfection, or ODN2006 gymnotic), or treated with ASO at a concentration of 1 μM, 3 μM, or 10 μM for 24 hours (indicated on x-axes). Cytokine/chemokine levels were then analyzed using the MSD-U-Plex platform (indicated by y-axes). “GLS1 ASO” comprises the nucleotide sequence of SEQ ID NO: 233, a gapmer structure, and the chemical modifications as set forth in Columns A and C of row 234 of Table 1. Plots show mean+/−standard error. Each dot represents an individual donor. N=4 donors (2 male and 2 female). FIG. 10A shows analyses of IFN-a2a levels. FIG. 10B shows analyses of IFN-b levels. FIG. 10C shows analyses of IL-1B levels. FIG. 10D shows analyses of IL-6 levels. FIG. 10E shows analyses of IL-10 levels. FIG. 10F shows analyses of IP-10 levels. FIG. 10G shows analyses of MCP-1 levels. FIG. 10H shows analyses of MIP-1a levels. FIG. 10I shows analyses of MIP-1b levels. FIG. 10J shows analyses of TNF-a levels.

FIG. 11 shows a non-limiting example of a study design wherein non-human primate subjects were administered a series of four intrathecal injections of either vehicle (artificial CSF) or ASO at a dose of 80 mg (20 mg+20 mg+20 mg+20 mg). Each intrathecal injection was performed two weeks apart (days 0, 14, 28, and 42).

FIG. 12 shows ASO levels in dorsal root ganglion (DRG), hippocampus, lumbar spinal cord, motor cortex, prefrontal cortex, and temporal cortex samples obtained from injected non-human primate subjects and assessed by liquid chromatography-tandem mass spectrometry (LC-MS/MS). Non-human primate subjects received ASO at a dose of 80 mg (20 mg+20 mg+20 mg+20 mg) by intrathecal injection as illustrated in FIG. 11. The indicated samples were obtained at two weeks post-final injection of ASO (day 56). Each dot represents a sample from obtained from a different non-human primate subject. N=2-3 for each of the indicated groups of samples. Bars show mean+/−standard error of the mean. “GLS1 ASO” comprises the nucleotide sequence of SEQ ID NO: 233, a gapmer structure, and the chemical modifications as set forth in Columns A and C of row 234 of Table 1.

### DETAILED DESCRIPTION

Aspects of the disclosure relate to compositions and methods for modulating a level, transcription, splicing, and/or translation of one or more RNA transcripts (e.g., mRNA transcripts) in a cell or subject. The disclosure is based, in part, on isolated nucleic acids that bind to mRNA transcripts of genes involved in glutamate signaling, for example a gene encoding glutaminase (GLS1). In some embodiments, compositions of the disclosure are useful for treating diseases or disorders associated with dysregulation of glutamate signaling, such as schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.). In some embodiments, compositions of the disclosure are useful for reducing neuroinflammation in a subject in need thereof.

### Glutamate Signaling

Glutamate is the anion of glutamic acid, and acts as an excitatory neurotransmitter that is involved in modulating a variety of biological processes. Glutamate signals through three different receptor types: AMPA receptors, NMDA receptors, and metabotropic glutamate receptors. Glutamate signaling through NMDA receptors is important for controlling synaptic plasticity and mediating learning and memory functions.

After binding to a receptor (e.g., an NMDA receptor) in the synaptic junction, glutamate is taken up by astrocytes where it is converted to glutamine by the glutamine synthetase pathway. Glutamine is then transported back into presynaptic neurons, where glutaminase (GLS1) converts the glutamine to glutamate, which can then subsequently be released by synaptic vesicles. There are two different types of glutaminase: “kidney-type” (also referred to as “GLS1”), and “liver-type” (also referred to as GLS2), although both types are expressed in CNS (e.g., brain) tissue. In some embodiments, a glutaminase protein is a GLS1 glutaminase protein.

Glutamate signaling also plays a major role in psychiatric diseases and disorders, and genes associated with glutamate signaling have traditionally served as an important therapeutic target for managing these indications. Accordingly, aspects of the disclosure relate to compositions for altering a level, transcription, splicing, and/or translation of genes associated with glutamate signaling.

A “gene associated with glutamate signaling” refers to a gene encoding a gene product (e.g., an mRNA, protein, etc.) that is genetically, biochemically, or functionally associated with the release, reuptake, and or interaction between glutamate and a glutamate receptor (e.g., an NMDA receptor, for example an NMDA receptor) in a cell or subject. In some embodiments, a gene associated with glutamate signaling is GLS1.

In some embodiments, a gene associated with glutamate signaling encodes an mRNA encoding a glutaminase (GLS1) protein. In humans, GLS1 is encoded by the GLS1 gene, located on chromosome 2 (e.g., encoded by Ensembl ID NO: ENSG00000115419, Chromosome 2: 190,880,821-190,965,552 forward strand). In some embodiments, GLS1 encodes a peptide that is represented by NCBI Reference Sequence NP_055720.3. In some embodiments, a GLS1 gene encodes an mRNA comprising the sequence set forth in NCBI Reference Sequence NM_014905.5. In some embodiments, an mRNA is encoded by a GLS1 gene and comprises one of the sequences set forth below:

NM_014905.5
(SEQ ID NO: 304)
AGTGCGGAGCCTTAGGCGGAGCGAAGAGAACCGGTCGCGGCAATCCTAGCGCGCAGCAGCAGCAGCAGCAGCAGCAG
CAGCAGCAGCAGCAGCAGCAGCACCCGCATCCGCTGCGGGAGTCCGAGCCGGAACCACACCCAAGTAGCTGCCCTTT
CCTCTTCTGTCATCTCACCGCCCCACCACAGACCGCGTTCCCCGAGGAAACCGGCCGCCCACGCCCGGAGCATCCTC
CCCTGTTGAGCGGGCGCTGACGGACCCGGCGGCATGATGCGGCTGCGAGGCTCGGGGATGCTGCGGGACCTGCTCCT
GCGGTCGCCCGCCGGCGTGAGCGCGACTCTGCGGCGGGCACAGCCCTTGGTCACCCTGTGCCGGCGTCCCCGAGGCG
GGGGACGGCCGGCCGCGGGCCCGGCTGCCGCCGCGCGACTCCACCCGTGGTGGGGCGGGGGCGGCTGGCCGGCGGAG
CCCCTCGCGCGGGGCCTGTCCAGCTCTCCTTCGGAGATCTTGCAGGAGCTGGGCAAGGGGAGCACGCATCCGCAGCC
CGGGGTGTCGCCACCCGCTGCCCCGGCGGCGCCCGGCCCCAAGGACGGCCCCGGGGAGACGGACGCGTTTGGCAACA
GCGAGGGCAAAGAGCTGGTGGCCTCAGGTGAAAATAAAATAAAACAGGGTCTGTTACCTAGCTTGGAAGATTTGCTG
TTCTATACAATTGCTGAAGGACAAGAGAAAATACCTGTTCATAAATTTATTACAGCACTCAAATCTACAGGATTGCG
AACGTCTGATCCCAGGTTGAAAGAGTGTATGGATATGTTAAGATTAACTCTTCAAACAACATCAGATGGTGTCATGC
TAGACAAAGATCTTTTTAAAAAATGTGTTCAGAGCAACATTGTTTTGTTGACACAAGCATTTAGAAGAAAGTTTGTG
ATTCCTGACTTTATGTCTTTTACCTCACACATTGATGAGTTATATGAAAGTGCTAAAAAGCAGTCTGGAGGAAAGGT
TGCAGATTATATTCCTCAACTGGCCAAATTCAGTCCCGATTTGTGGGGTGTGTCTGTTTGTACAGTAGATGGACAGA
GGCATTCTACTGGAGATACCAAAGTTCCCTTCTGTCTTCAGTCCTGTGTAAAACCTTTGAAATATGCCATTGCTGTT
AATGATCTTGGAACTGAATATGTGCATCGATATGTTGGAAAAGAGCCGAGTGGACTAAGATTCAACAAACTATTTTT
GAATGAAGATGATAAACCACATAATCCTATGGTAAATGCTGGAGCAATTGTTGTGACTTCACTAATAAAGCAAGGAG
TAAATAATGCTGAAAAATTTGACTATGTCATGCAGTTTTTGAATAAGATGGCTGGTAATGAATATGTTGGATTCAGT
AATGCAACGTTTCAGTCTGAAAGAGAAAGTGGAGATCGAAATTTTGCAATAGGATATTACTTAAAAGAAAAGAAGTG
TTTTCCAGAAGGCACAGACATGGTTGGTATATTAGACTTCTACTTCCAGCTGTGCTCCATTGAAGTGACTTGTGAAT
CAGCCAGTGTGATGGCTGCGACACTGGCTAATGGTGGTTTCTGCCCAATTACTGGTGAAAGAGTACTGAGCCCTGAA
GCAGTTCGAAATACATTGAGTTTGATGCATTCCTGTGGCATGTATGACTTCTCAGGGCAGTTTGCTTTCCATGTTGG
TCTTCCTGCAAAATCTGGAGTTGCTGGGGGCATTCTTTTAGTTGTCCCCAATGTTATGGGTATGATGTGCTGGTCTC
CTCCTCTGGATAAGATGGGCAACAGTGTTAAGGGAATTCACTTTTGTCACGATCTTGTTTCTCTGTGTAATTTCCAT
AACTATGATAATTTGAGACACTTTGCAAAAAAACTTGATCCTCGAAGAGAAGGTGGTGATCAAAGGGTAAAGTCAGT
GATAAATCTTTTGTTTGCTGCATATACTGGAGATGTGTCTGCACTTCGAAGATTTGCTTTGTCAGCTATGGACATGG
AACAGCGGGACTATGATTCTAGAACAGCACTCCATGTAGCTGCTGCAGAGGGTCATGTTGAAGTIGTTAAATTTTTG
CTGGAAGCCTGCAAAGTAAACCCTTTCCCCAAGGACAGGTGGAATAACACTCCCATGGATGAAGCACTGCACTTTGG
ACACCATGATGTATTTAAAATTCTCCAAGAATACCAAGTCCAGTACACACCTCAAGGAGATTCTGACAACGGGAAGG
AAAATCAAACCGTCCATAAGAATCTTGATGGATTGTTGTAATGGTCTCAAATCCCAAGATTTAAATCACTTACCTAT
TTAATTGTGGAAAATGATTATGAAGAACATGTGTATTTCTATCTGGTAGTGATGTATATTTTACATTTGTCATTTCA
GTGTTACTGGAGTTTTCTTCATTGTGCACACAGGACAAATCTGATCTCTTTGGGAAAAAATAGAAATAAAACAATCT
CCCTCCATAATGTGAGCAATATTACCTCGTGCATTGTATAATTTGATGTAAAAGAAATAGTTACCAATGCTAGCTTG
TGTGGTCTTCCATGATTTATTTGTGTTTTGTGAATTTTCAATTTATGGTGATGATCTGCTGATATGCATTTATAAAG
TAAGCTCTGTTGTACAGTCTGTCCAAATGGGTCAAGGTTGCCTTTAGAAGCAAATAGTGTGATTTTCAAGACTTCAA
ATACAAATTTAGTTTAAGTGTTTGAACAACTATATGCACTTACGGTTGTGTGTTTAAAATGTCTCTCTCACCCCCTA
GCTTCATGATGTGACTCTTAAAAAACTATAATAGTTAACAACTGTTAGTAAGATAGACCAATTCTGATTAGACTTTA
TCAGGGAATCTGTTTAAGATATGTTTGGTGACCAAAACGTATGTGTGAATGTAGTTATAATGCTTTTGAAAAATTTT
CCTTTTTCTATATCCCCTTAGTCCAGCCTCTCTTCTCAGACATTTAGCTATCTGCCTCTTTCCTTTAGCTGGGAAAG
TGAGAGCTGGCATACTATGCAGTTTTTATGTTTTCCATAGTAAGTCAGAAAATGCCTCCTATTTCTGGCATCAGAAC
TTTGCCATTTGTCTACAGAAGACGAACCAGAGACAAAATTACTAAGTATAAATTAGTCAAGTTTATCAGTCTAAAAA
ACGAAGGGATGTGCAACTGCAGCTCTTTAAGAAGTTTTTTTTTTTTAGCTTCTAGGGTAAAGATAAATTCAGAAATG
CTCTAAGCTACCAAAGTTATTCTGAAAGTATGGGAACTGCTACAACTAACAAACATTTGTTTCCAAGCCTGTCATTA
AGAGTCTGCATCAAGAGATTTGTCCTCCTTGGGGGACCACTGGATCATTCCAGATTTCTTGTGATTTTTCTATTGTG
TAATTCTTGGTGGGCTCTGTAGTTTAATAATAAGAAAAAGGCCATTTCATTTTAAATTGTGACCTATAATTCTTTGT
CTTGGGTTGGTAATTCAGGATTCATTTGGAAAGTGGGTAAAAGGGGCTTCAAAAAACGGATAGAACAGGATTTTCTA
GGAGTTACACATACATTTTATCCTGTCATACCTCGAGATAAAGTGGCATGTTAGTGAGGAGTTCTGATATTAAGCAC
ACACACACATGCACACAAATGGACTTCTCTGAAGCTGTGTTTAGTGAAATGAGCTCAAGTACATGAATGTTAGTTGT
TATCACATACAGCAAATTCCTTTTTTTTTCTTTTTCTATGAGCACACTCTGCTGCTTCTAAACTTTACATGCCTGAT
GGCACCTTACTCCAGCAGCCTCCAGGTGCTTTCATTTTCACTTCCAGTCTAAGCCAGTGGCTCCTGCCACTGCCCTC
CCATTACCTAGATGGCACCTCCTTTGGTGAAACCACGGCCAATGTTCCTTAGCTGCACCAGGCCCGAAGCTGTTCCC
ATGCTTGAGCTTCCATGGGGAGGATGCTGAGTGAGCAGTTTCCTACCCCGTGGATCTAGCAAGCCATGGAGACAGGT
AGCATTTGTAAGATGCTGCACAGGAGCAGCATTATCCCCAAAGATATTACAGGGTAGACACGTTTTAACTGAAATCA
ATCAAGATAACTTTATTCAAAGAGCAGCCCGCTTTGTGTGACTAAAATGAAACAAGACAGTTGAATTGTGTGACTTG
AAGATTACCAATGATTTTGAGGCTTTTCTATAATAAAAAGAGGTTCTAACCATTATTTGGGAACAAAGAGAGTTTTC
ATCTTTTTTCAGATCAAAACCATTCTGTAAAATCTTTGTTGTTTAATTAAATGTGCCGTTATTTACCCCTGATGTTA
TTTATGACTATGTGCCGATTCCTGCTCGGGCTGTTTGCTGTTGGCTGGTAATAATATATTTGATTTAAATGCTGTTG
ACTGTGCTATTAACTGCTGCCGTCAGTAAACTCCAAAGATCTTTTTGTTTTGGCTTTAGTATCATATGTGCTTTTTC
TGTATCCTGAGCGCTCTATATGATCATGTTAATTTAAAGCTTTATACACATTGTTGTTTTTGCTGGTCTCATCTTTG
GTAATATGCTATACCCCACTGCTGCCCGACACTGCCCTTTAGCTGCAGAGCTGGATTAGCTGTTGACCATTIGATGC
TGTTGTCTGTCTGGCAGGGACTGAATGACCTGATGTCAGATTTAGATTCTTCCTGGGGATTACACAGCTATGAATGT
ATTTGCTTCTAAAACCTCCCAAAGTGAATCTAATCTTAAAACTACAAGTTGTAAGTATTCTGAAATTGGGAAACATT
TATTTTAAATGCAATCAGGTAGTGTTGCTTTTTACAGCATAATAAATATATGTATCAAAAAAAAAA

The skilled artisan recognizes that when referring to a gene sequence encoding an mRNA, the sequence of the mRNA is identical to the recited gene sequence, except that each instance of “T” is replaced with “U”.

Aspects of the disclosure relate to methods for reducing glutamate signaling in subjects having certain psychiatric diseases and disorders. In some embodiments, the subjects do not have any mutations in either allele of their GLS1 gene (e.g., the subjects have wild type GLS1 protein).

However, in some embodiments, a GLS1 gene (or an mRNA encoded by a GLS1 gene) comprises one or more nucleotide substitutions, one or more nucleotide insertions, and/or one or more nucleotide deletions relative to a wild type GLS1 gene (or mRNA encoded by a wild type GLS1 gene), and may be referred to as a “mutant” GLS1 gene or a GLS1 variant. The number of nucleotide substitutions, nucleotide insertions, and/or nucleotide deletions in a GLS1 variant may vary. In some embodiments, a GLS1 variant comprises between 1 and 20, 5 and 10, 2 and 15, 10 and 30, or 20 and 100 nucleotide substitutions, nucleotide insertions, and/or nucleotide deletions relative to a wild type GLS1 gene (or mRNA encoded by a wild type GLS1 gene). In some embodiments, the one or more nucleotide substitutions, one or more nucleotide insertions, and/or one or more nucleotide deletions results in an amino acid substitution in the protein encoded by the GLS1 variant. In some embodiments, the one or more nucleotide substitutions, one or more nucleotide insertions, and/or one or more nucleotide deletions results in a nonsense mutation (e.g., insertion of a premature stop codon) in an mRNA encoded by the GLS1 variant.

In some embodiments, the one or more nucleotide substitutions, one or more nucleotide insertions, and/or one or more nucleotide deletions results in a frameshift mutation of the GLS1 variant relative to a wild type GLS1 gene. In some embodiments, a mutation or mutations present in a GLS1 variant result in the production of one or more splice variants of GLS1 mRNA. A “splice variant” may refer to a mRNA resulting from one or more mutations in a DNA sequence that occur at the boundary of an exon and an intron (splice site) of a gene. Splice site mutations generally disrupt RNA splicing and result in the loss of exons or the inclusion of introns and an altered protein-coding sequence (e.g., a “splice variant”).

Aspects of the disclosure relate to isolated nucleic acids, for example RNA processing modulators (e.g., ASOs) that bind to one or more target regions of an mRNA encoded by a gene associated with glutamate signaling. In some embodiments, the isolated nucleic acids bind to more or more splice variants of a GLS1 gene (e.g., a human GLS1 splice variant). In some embodiments, an isolated nucleic acid described by the disclosure binds to a region of a GLS1 splice variant (e.g., mRNA encoded by a GLS1 variant) selected from an untranslated region (UTR). In some embodiments, the UTR is a 5′ UTR. In some embodiments, the UTR is a 3′ UTR. In some embodiments, the UTR is an intron. In some embodiments, an isolated nucleic acid described by the disclosure binds to an intron-exon boundary of a GLS1 splice variant (e.g., mRNA encoded by a GLS1 variant). An intron-exon boundary refers to a contiguous nucleotide sequence that includes portions of an intron and exon that are adjacent to one another in the mRNA transcript. In some embodiments, an isolated nucleic acid (e.g., antisense oligonucleotide) binds to an mRNA expressed from a particular allele of GLS1 (e.g., binds to a target mRNA in an allele-specific manner).

### Isolated Nucleic Acids

In some embodiments of the present disclosure, a nucleic acid is an isolated nucleic acid. In some cases, nucleic acids are alternatively referred to as oligonucleotides. In some embodiments, an isolated nucleic acid comprises DNA (e.g., deoxyribonucleotides). In some embodiments, an isolated nucleic acid comprises RNA (e.g., ribonucleotides). In some embodiments, an isolated nucleic acid comprises both DNA (e.g., deoxyribonucleotides) and RNA (e.g., ribonucleotides), such as an isolated nucleic acid comprising a gapmer structure that comprises a region of deoxyribonucleotides which are flanked by regions of ribonucleotides. An isolated nucleic acid may be single stranded or double stranded. In some embodiments, the isolated nucleic acid is an RNA oligonucleotide. In some embodiments, the isolated nucleic acid is a single stranded RNA oligonucleotide (which may also be referred to as a single stranded RNA polynucleotide).

As used herein, the term “isolated” means artificially produced. Artificial production of an isolated nucleic acid may be achieved, for example, through amplification in vitro through polymerase chain reaction (PCR), recombinant cloning, or chemical synthesis. Methods of synthesizing isolated nucleic acids, for example RNAs, are known in the art, for example as described by Soukchareun et al. Preparation and characterization of antisense oligonucleotide-peptide hybrids containing viral fusion peptides. Bioconjug Chem. 1995 January-February; 6(1):43-53. doi: 10.1021/bc00031a004. PMID: 7711103.

The length of an isolated nucleic acid may vary. In some embodiments, an isolated nucleic acid (e.g., a single stranded RNA) is 10, 15, 20, 25, 30, 40, 45, 50, 55, 60, 65, 70, 75, 80, 85, 90, 95, or up to 120 nucleotides in length. In some embodiments, an isolated nucleic acid ranges from about 1 to 100, 2 to 30, 5 to 20, 10 to 40, or 20 to 80 nucleotides in length. In some embodiments, an isolated nucleic acid is between 10 and 50 nucleotides in length. In some embodiments, an isolated nucleic acid comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, an isolated nucleic acid is more than 50 nucleotides in length (e.g., 60, 70, 80, 90, 100, etc., nucleotides in length). In some embodiments, an isolated nucleic acid is no greater than 200 nucleotides in length. In some embodiments, an isolated nucleic acid comprises a nucleotide sequence that encodes a full length, wild type GLS1 protein.

In some embodiments, an isolated nucleic acid of the disclosure comprises an antisense oligonucleotide comprising the sequence set forth in any one of SEQ ID NOs: 1-303 (provided in Column A of Table 1). In some embodiments, an isolated nucleic acid of the disclosure comprises an antisense oligonucleotide comprising at least 15 nucleotides (e.g., at least 15, at least 16, at least 17, at least 18, at least 19, or at least 20 nucleotides) of the any one of the sequences set forth in SEQ ID NOs: 1-303 (provided in column A of Table 1).

In some embodiments of the present invention, an isolated nucleic acid is modified (e.g., comprises one or more modifications, for example chemical modifications). A modified nucleic acid may refer to an oligonucleotide that has been structurally altered in a non-natural manner (e.g., a manner that does not occur in nature). Nucleic acid modifications may be used to endow the nucleic acid with specific functional characteristics relative to unmodified nucleic acids. In some embodiments, modification of an isolated nucleic acid promotes binding of the isolated nucleic acid to a target molecule or increases stability of the isolated nucleic acid (e.g., makes the isolated nucleic acid resistant to enzymatic degradation).

In some embodiments, the one or more modifications is between 1 and 50 modifications, 2 and 20, 5 and 30, 10 and 40, or 15 and 50 modifications. In some embodiments, an isolated nucleic acid comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications. In some embodiments, an isolated nucleic acid comprises more than 50 modifications (e.g., up to 60, 70, 80, 90, or 100, etc., modifications). In some embodiments, an isolated nucleic acid comprises chemical modifications on each nucleotide and each sugar-phosphate backbone linkage. Such a modified isolated nucleic acid may be referred to as a “fully modified” isolated nucleic acid. In some embodiments, not all nucleotides of an isolated nucleic acid are modified.

A chemical modification may comprise a modification of a nucleobase or a nucleotide, and/or a modification of a sugar-phosphate backbone (e.g., modification of one or more sugar-phosphate backbone linkages).

In some embodiments, an isolated nucleic acid of the disclosure comprises one or more chemical modification(s) listed in Column C of Table 1.

In some embodiments, an isolated nucleic acid comprises one or more modifications to a 5′ carbon atom (e.g., a 5′-carbon atom of a sugar) and/or one or more modifications to a 5-carbon of a nucleobase. Examples of modifications include, but are not limited to, 5-(2-amino)propyl uridine, 5-bromo uridine, 5-propyne uridine, 5-propenyl uridine, 5-carboxymethylaminomethyl-2-thiouracil, and 5-carboxymethylaminomethyl uracil. In other embodiments, the nucleic acid modification is targeted to the 6-carbon atom of a nucleobase. In some embodiments, an isolated nucleic acid comprises one or more modifications to a 6-carbon atom (e.g., a 6-carbon atom of a nucleobase) for example a 6-(2-amino)propyl uridine. In some embodiments, an isolated nucleic acid comprises one or more modifications to an 8-carbon atom (e.g., an 8-carbon atom of a nucleobase). Examples of 8 modifications include, but are not limited to, 8-bromo guanosine, 8-chloro guanosine, and 8-fluoroguanosine.

In some embodiments, an isolated nucleic acid comprises one or more modifications to a 2′ carbon of the sugar group. Examples of modified sugar groups include, but are not limited to, D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-2-O-methoxyethoxy, 2′-allyloxy (—OCH2CH═CH2), 2′-propargyl, 2′-propyl, ethynyl, ethenyl, propenyl, and cyano and the like. In some embodiments, a modified sugar moiety comprises a hexose and incorporated into an oligonucleotide as described (Augustyns, K., et al., Nucl. Acids. Res. 18:4711 (1992)). Other examples of 2′ modifications include, but are not limited to, substitutions of the bound OH group with H, OR, R, F, Cl, Br, I, SH, SR, NH, NHR, NR, COOR, or OR, wherein R is a substituted or unsubstituted aliphatic group. Other 2′ modifications are found in the art. The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties.

In some embodiments, an isolated nucleic acid modification a sugar-phosphate backbone modification. One example of a phosphate group modifications is substitution of an oxygen atom with a sulfur atom. In other embodiments, the backbone of the nucleic acid is modified. Examples of backbone modifications include, but are not limited to, phosphorothioate, borano-phosphate, alkyl phosphonate nucleic acid, peptide nucleic acid, and morpholino. Morpholino backbones are described, for example by Corey and Abrams Genome Biol. 2001; 2(5): reviews1015.1-reviews1015.3.

Other examples of modified bases include N4,N4-ethanocytosine, 7-deazaxanthosine, 7-deazaguanosine, 8-oxo-N6-methyladenine, 4-acetylcytosine, dihydrouracil, inosine, N6-isopentenyl-adenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 2-methylthio-N6-isopentenyladenine, pseudouracil, 5-methyl-2-thiouracil, 2-thiouracil, 4-thiouracil, 2-thiocytosine, and 2,6-diaminopurine. Other examples of nucleic acid modifications are described for example by Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, Duffy. BMC Bio. 2020 Sep. 2(8):112, and U.S. Pat. No. 5,684,143.

Additional modifications of isolated nucleic acids (e.g., ASOs) are described by Duffy et al. BMC Biology volume 18, Article number: 112 (2020), the entire contents of which are incorporated herein by reference.

In some embodiments, an isolated nucleic acid of the disclosure comprises a nucleic acid sequence from Column A of Table 1 and one or more chemical modifications (or combinations of chemical modifications) from Column C of Table 1, optionally where Columns A and C are from the same row of Table 1.

### RNA Processing Modulators

Aspects of the disclosure relate to compositions (e.g., isolated nucleic acids, agents, etc.) that modulate mRNAs encoded by genes associated with glutamate signaling. In some embodiments, the gene associated with glutamate signaling is GLS1 (e.g., a human GLS1 gene). In some embodiments, a composition comprises an RNA processing modulator. As used herein, an “RNA processing modulator” or “RPM” refers to an agent that binds to, and up-regulates, down-regulates, or otherwise change function or activity, of a target mRNA (e.g., an mRNA encoded by a gene associated with glutamate signaling, such as GLS1, or a gene product, such as a protein encoded by the mRNA) by affecting transcription, levels, splicing, and/or translation of the mRNA. An RNA processing modulator may be an isolated nucleic acid or ASO as described herein. In some embodiments, an RNA processing modulator is an isolated nucleic acid that affects transcription, levels, splicing, and/or translation of a target mRNA (e.g., an mRNA encoded by a GLS1 gene). In some embodiments, an RNA processing modulator is an antisense oligonucleotide that affects transcription, levels, splicing, and/or translation of a target mRNA (e.g., an mRNA encoded by a GLS1 gene). In some embodiments, an mRNA (e.g., a target mRNA, such as an mRNA encoded by a GLS1 gene) is a pre-mRNA (e.g., an RNA that has been transcribed from a gene, such as a GLS1 gene, but has not been processed to remove introns, for example by splicing). In some embodiments, an mRNA is a mature mRNA that has been processed (e.g., an mRNA transcribed from a GLS1 gene and that has undergone processing).

In some embodiments, an RNA processing modulator upregulates transcription, levels, splicing, and/or translation of a target mRNA. Upregulation of transcription, levels, splicing, and/or translation may comprise binding to a regulatory region (e.g., an untranslated region, such as a 5′ UTR or 3′ UTR) of a target mRNA and reducing non-productive splicing or translation initiation from alternative start codons present in the target mRNA, for example through steric blocking of non-productive splice site(s) or alternative start codons (such as “upstream alternative start codons” located in the 5′ UTR of the target mRNA), or causing a mRNA frameshift (e.g., a splice variant) resulting in translation of a protein variant from the target mRNA that lacks one or more inhibitory domains.

The amount of upregulation of transcription, levels, splicing, and/or translation mediated by an RNA processing modulator may vary. In some embodiments, an RNA processing modulator increases transcription, levels, splicing, and/or translation of a target mRNA transcript (e.g., increases relative to a cell or subject prior to the administration of the RPM, or increases relative to a control cell or subject) between 1-fold and 100-fold, 2-fold and 10-fold, 5-fold and 20-fold, 10-fold and 30-fold, 20-fold and 50-fold, or 25-fold and 100-fold, or any value therebetween. In some embodiments, an RNA processing modulator increases transcription, levels, splicing, and/or translation of a target mRNA transcript more than 100-fold, for example at least 200-fold, 400-fold, 500-fold, or 1000-fold. In some embodiments, an RNA processing modulator increases transcription, levels, splicing, and/or translation of a target mRNA transcript no more than 1000-fold. In some embodiments, upregulation of a level, transcription, splicing, and/or translation of a target mRNA is useful to increase expression of a desired (e.g., wild-type) allele encoding the target mRNA.

In some embodiments, an RNA processing modulator downregulates transcription, levels, splicing, and/or translation of a target mRNA. Downregulation of transcription, levels, splicing, and/or translation may comprise binding to a regulatory region (e.g., an untranslated region, such as a 5′ UTR or 3′ UTR) of a target mRNA and blocking transcription the target mRNA, for example through steric blocking of a transcription initiation site, binding to an mRNA and subsequently initiating RNAse H-mediated degradation (e.g., in the context of a ‘gapmer’ RNA processing modulator), or causing an mRNA frameshift (e.g., a splice variant) resulting in translation of a protein variant from the target mRNA that is inactive, or has reduced function or activity (e.g., enzymatic activity, the ability to interact with other proteins to form protein complexes, etc.). In some embodiments, the resulting protein variant is a dominant negative protein variant. In some embodiments, downregulation of a level, transcription, splicing, and/or translation of a target mRNA is useful to increase expression of an undesirable (e.g., mutant, or disease-associated) allele encoding a target mRNA.

The amount of downregulation of transcription, levels, splicing, and/or translation mediated by an RNA processing modulator may vary. In some embodiments, an RNA processing modulator decreases transcription, levels, splicing, and/or translation of a target mRNA transcript between 1-fold and 100-fold, 2-fold and 10-fold, 5-fold and 20-fold, 10-fold and 30-fold, 20-fold and 50-fold, or 25-fold and 100-fold, or any value therebetween. In some embodiments, an RNA processing modulator decreases transcription, levels, splicing, and/or translation of a target mRNA transcript more than 100-fold, for example at least 200-fold, 400-fold, 500-fold, or 1000-fold. In some embodiments, an RNA processing modulator decreases transcription, levels, splicing, and/or translation of a target mRNA transcript no more than 1000-fold.

An RNA processing modulator may alter the number and/or character of splice variants of a target mRNA. In some embodiments, an RNA processing modulator increases (relative to natural transcription or translation of a target mRNA) the number of different splice variants of an mRNA, or the ratio between different splice variants of an mRNA. In some embodiments, an RNA processing modulator decreases (relative to natural transcription or translation of a target mRNA) the number of different splice variants of an mRNA, or the ratio between different splice variants of an mRNA. In some embodiments, contacting a target mRNA with an RNA processing modulator results in 1, 2, 3, 4, 5, 6, 7, 8, 9, 10, or more splice variants of the target mRNA being transcribed and/or translated. In some embodiments, contacting a target mRNA with an RNA processing modulator results in a single splice variant of the target mRNA being transcribed and/or translated.

The binding location of an RNA processing modulator may vary. In some embodiments, an RNA processing modulator affects splicing of the target mRNA. For example, an RNA processing modulator may bind to the target mRNA at a splice junction (e.g., a location spanning an intron-exon boundary) and mediate skipping of one or more exons in the mRNA transcript. In some embodiments, skipping of one or more exons in the target mRNA results in production of a truncated protein variant of the protein encoded by the target mRNA. In another example, an RNA processing modulator may bind to the target mRNA at a splice junction and mediate alternative splicing in which an intron is translated, and a protein variant of the target gene is produced. In some embodiments, an RNA processing modulator binds a target mRNA at a location comprising a coding sequence (e.g., a protein coding sequence or an exon).

In some embodiments, an RNA processing modulator comprises an agent selected from the group consisting of nucleic acid, peptide (including polypeptide), and small molecule. Examples of small molecule RNA processing inhibitors include but are not limited to translational readthrough-inducing drugs (TRIDs), such as certain aminoglycosides, non-aminoglycoside antibiotics (e.g., negamycin), ataluren (PTC124), and amlexanox. Examples of peptides include but are not limited to activator proteins (e.g., transcription factors), suppressor proteins (e.g., inducible cAMP early repressor (ICER), bZIP repressor, SP1 repressor, certain histone deacetylases, etc.), antibodies, etc. Examples of nucleic acids include but are not limited to suppressor tRNAs, dsRNA, siRNA, micro-RNA (miRNA), artificial miRNA (ami-RNA), aptamers, and antisense oligonucleotides. In some embodiments, an RNA processing modulator comprises an antisense oligonucleotide (ASO).

As used herein, the term, “antisense nucleic acid,” or “ASO” refers to a single stranded nucleic acid that has sequence complementarity to a target sequence and is specifically hybridizable, e.g., under stringent conditions, with a nucleic acid having the target sequence. An antisense nucleic acid is specifically hybridizable when binding of the antisense nucleic acid to the target nucleic acid is sufficient to produce complementary base pairing between the antisense nucleic acid and the target nucleic acid, and there is a sufficient degree of complementarity to reduce or avoid non-specific binding of the antisense nucleic acid to non-target nucleic acid under conditions in which specific binding is desired, e.g., under physiological conditions in the case of in vivo assays or therapeutic treatment, and in the case of in vitro assays, under conditions in which the assays are performed. In some embodiments, an ASO is chemically synthesized. An ASO may be a DNA polynucleotide, an RNA polynucleotide, or a DNA/RNA polynucleotide (e.g., an ASO comprising a gapmer structure that comprises a region of deoxyribonucleotides flanked by regions comprising ribonucleotides).

Complementary refers to the capacity for precise pairing between two nucleotides. For example, if a nucleotide at a certain position of an antisense nucleic acid is capable of hydrogen bonding with a nucleotide at the corresponding position of a target nucleic acid (e.g., target RNA), then the antisense nucleic acid and target nucleic acid are considered to be complementary to each other at that position. The antisense nucleic acid and target nucleic acid are complementary to each other when a sufficient number of corresponding positions in each molecule are occupied by nucleotides that can hydrogen bond with each other through their bases. Thus, “complementary” is a term that is used to indicate a sufficient degree of complementarity or precise pairing such that stable and specific binding occurs between the antisense nucleic acid and target nucleic acid. However, it should be appreciated that 100% complementarity is not required. For example, in some embodiments, an antisense nucleic acid (e.g., an oligonucleotide) may be at least 80% complementary to (e.g., at least 85%, 90%, 91%, 92%, 93%, 94%, 95%, 96%, 97%, 98%, 99% or 100% complementary to) the consecutive nucleotides of a target nucleic acid (e.g., a target nucleic acid comprising an mRNA sequence encoded by SEQ ID NO: 304).

Sequence identity, including determination of sequence complementarity for nucleic acid sequences, may be determined by sequence comparison and alignment algorithms known in the art. To determine the percent identity of two nucleic acid sequences, the sequences are aligned for optimal comparison purposes (e.g., gaps can be introduced in the first sequence or second sequence for optimal alignment). The nucleotides at corresponding nucleotide positions are then compared. When a position in the first sequence is occupied by the same residue as the corresponding position in the second sequence, then the molecules are identical at that position. In some embodiments, the percent identity between the two sequences is a function of the number of identical positions shared by the sequences (e.g., % homology=# of identical positions/total # of positions×100), optionally penalizing the score for the number of gaps introduced and/or length of gaps introduced.

In some embodiments, an antisense oligonucleotide has a length in a range of 5 to 40 nucleotides, 5 to 30 nucleotides, 10 to 30 nucleotides, 10 to 25 nucleotides, or 15 to 25 nucleotides. In some embodiments of the disclosure, an antisense oligonucleotide comprises a length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides.

In some embodiments, an antisense nucleic acid comprises a region of complementarity that is perfectly complementary to a portion of a target nucleic acid (e.g., 100% of the nucleotides of the ASO hybridize to the nucleotides of the target RNA, such as a target mRNA (e.g., an mRNA sequence encoded by SEQ ID NO: 304)). However, it should be appreciated that in some embodiments, an antisense nucleic acid comprises less than 100% sequence complementarity with a target nucleic acid (e.g., 50%, 60%, 70%, 80%, 90%, 95%, or 99% of the nucleotides of the ASO hybridize to the nucleotides of the target RNA, such as a target mRNA (e.g., an mRNA sequence encoded by SEQ ID NO: 304)). In addition, to minimize the likelihood of off-target effects, an antisense nucleic acid may be designed to ensure that it does not have a sequence (e.g., of 5 or more consecutive nucleotides) that is complementary with an off-target nucleic acid (e.g., an mRNA that is not transcribed from a GLS1 gene).

In some embodiments, an antisense oligonucleotide comprises a region of complementarity with an mRNA encoded by (e.g., transcribed from) a GLS1 gene. In some embodiments, an antisense nucleic acid oligonucleotide comprises a region of complementarity with an mRNA encoded by the sequence as set forth in SEQ ID NO: 304. In some embodiments, the region of complementarity of the antisense nucleic acid hybridizes with at least 6, e.g., at least 7, at least 8, at least 9, at least 10, at least 15 or more consecutive nucleotides of a target nucleic acid (e.g., an mRNA encoded by the sequence set forth in SEQ ID NO: 304). In some embodiments, an antisense oligonucleotide comprises a region of complementarity with a 5′ UTR, 3′ UTR, an exonic sequence, a splice donor sequence, a splice acceptor sequence or a lariat branch point encoded by a human GLS1 gene. In some embodiments, an oligonucleotide binds to an mRNA expressed from a particular allele of GLS1 (e.g., binds to a target mRNA in an allele-specific manner).

In some embodiments, an antisense oligonucleotide comprises a region of complementarity with an mRNA encoded by (e.g., transcribed from) a GLS1 gene. In some embodiments, an antisense oligonucleotide comprises a region of complementarity with a pre-mRNA sequence encoded by a human GLS1 gene, for example (e.g., Ensembl ID NO: ENSG00000115419, Chromosome 2: 190,880,821-190,965,552 forward strand). In some embodiments, the region of complementarity of the antisense nucleic acid hybridizes with at least 6, e.g., at least 7, at least 8, at least 9, at least 10, at least 15 or more consecutive nucleotides of a target nucleic acid (e.g., a pre-mRNA encoded by Ensembl ID NO: ENSG00000115419, Chromosome 2: 190,880,821-190,965,552 forward strand). In some embodiments, the antisense oligonucleotide comprises a region of complementarity with at least 6, e.g., at least 7, at least 8, at least 9, at least 10, at least 15 or more consecutive nucleotides of an intron encoded by Ensembl ID NO: ENSG00000115419, Chromosome 2: 190,880,821-190,965,552 forward strand. The skilled artisan recognizes that the reverse strand of such a nucleic acid encoding a pre-mRNA transcript or mature mRNA transcript may also be targeted.

In some embodiments, an antisense oligonucleotide comprises a region of complementarity that is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 continuous nucleotides complementary with an mRNA encoded by the sequence as set forth in SEQ ID NO: 304. In some embodiments, an antisense oligonucleotide comprising a region of complementarity with an mRNA transcript encoded by SEQ ID NO: 304 comprises at least 60% sequence identity (e.g., 60-70%, 70-80%, 80-90%, 90-95%, or more than 95% sequence identity) to a nucleic acid sequence set forth in any one of SEQ ID NOs: 1-303, as recited in Column A of Table 1. In some embodiments, an antisense oligonucleotide comprises a sequence of 10 or more contiguous nucleotides (e.g., 10, 11, 12, 13, 14, 15, 16, 17, 18, or more contiguous nucleotides) of any one of the sequences set forth in SEQ ID NOs: 1-303, as recited in Column A of Table 1. In some embodiments, an antisense oligonucleotide comprises the nucleic acid sequence set forth in any one of SEQ ID NOs: 1-303, as recited in Column A Table 1. In some embodiments, an antisense oligonucleotide comprises a nucleotide sequence having one or more mismatches (e.g., one or more bases that is not complementary to the nucleotide at a given position of the target mRNA) relative to an mRNA transcript encoded by the sequence set forth in SEQ ID NO: 304. In some embodiments, an antisense oligonucleotide comprises a sequence having 1, 2, 3, 4, 5, 6, 7, 8, 9, or 10 mismatches relative to an mRNA transcript encoded by the sequence set forth in SEQ ID NO: 304. In some embodiments, an antisense oligonucleotide comprising one or more mismatches relative to an mRNA transcript encoded by SEQ ID NO: 304 comprises at least 60% sequence identity (e.g., 60-70%, 70-80%, 80-90%, 90-95%, or more than 95% sequence identity) to a sequence of 10 or more contiguous nucleotides of any one of the sequences set forth in SEQ ID NOs: 1-303, as recited in Column A of Table 1. In some embodiments, an antisense oligonucleotide comprising at least 60% sequence identity to a sequence of 10 or more contiguous nucleotides (e.g., 10, 11, 12, 13, 14, 15, 16, 17, 18, or more contiguous nucleotides) of any one of the sequences set forth in SEQ ID NOs: 1-303 differs at one or more nucleotide positions (e.g., 1, 2, 3, 4, 5, 6, 7, 8, 9, 10, or more nucleotide positions comprising a substitution, an insertion, or a deletion) relative to the sequence of 10 or more contiguous nucleotides of any one of the sequences set forth in SEQ ID NOs: 1-303, as recited in Column A of Table 1. In some embodiments, an antisense oligonucleotide comprising one or more mismatches relative to an mRNA transcript encoded by SEQ ID NO: 304 comprises at least 60% sequence identity (e.g., 60-70%, 70-80%, 80-90%, 90-95%, or more than 95% sequence identity) to a nucleic acid sequence set forth in any one of SEQ ID NOs: 1-303. In some embodiments, an antisense oligonucleotide comprising at least 60% sequence identity to a nucleic acid sequence set forth in any one of SEQ ID NOs: 1-303 differs at one or more nucleotide positions (e.g., 1, 2, 3, 4, 5, 6, 7, 8, 9, 10, or more nucleotide positions comprising a substitution, an insertion, or a deletion) relative to the nucleic acid sequence set forth in any one of SEQ ID NOs: 1-303.

In some embodiments, RNA processing modulators (e.g., antisense oligonucleotides) are provided in a homogeneous preparation, e.g., in which at least 85%, at least 90%, at least 95%, or at least 99% of the RNA processing modulators (e.g., antisense oligonucleotides) are identical. In some embodiments, a homogeneous preparation is stereo-pure (e.g., diastereomeric). For example, in some embodiments, homogeneous preparations of antisense oligonucleotides are provided in which at least 85%, at least 90%, at least 95%, or at least 99% of the oligonucleotides in the preparation are 10 to 25 nucleotides in length and comprise a region of complementarity that is complementary with at least 6 contiguous nucleotides of an mRNA transcript encoded by a GLS1 gene (e.g., a GLS1 gene encoding an mRNA comprising the nucleic acid sequence set forth in SEQ ID NO: 304). In some embodiments, RNA processing modulators (e.g., antisense oligonucleotides) are provided in a heterogeneous preparation, e.g., comprising at least 2, 3, 4, 5, 6, 7, 8, 9, 10, or more different RNA processing modulators (e.g., antisense oligonucleotides each targeting a different sequence of a GLS1 mRNA transcript).

RNA processing modulators (e.g., antisense oligonucleotides) of the disclosure may be modified to achieve one or more desired properties, such as, for example, improved cellular uptake, improved stability, reduced immunogenicity, improved potency, improved target hybridization, susceptibility to RNAse cleavage, etc. In some embodiments, an antisense nucleic acid is modified such that when present in a cell that contains a GLS1 gene, it is capable of hybridizing with RNA transcribed from the GLS1 gene without inducing cleavage of the RNA by an RNase. In some embodiments, an antisense nucleic acid is modified such that when present in a cell that contains a GLS1 gene, it is capable of hybridizing with RNA transcribed from the GLS1 gene and inducing cleavage of the RNA by an RNase.

RNA processing modulators (e.g., antisense oligonucleotides, e.g. a nucleic acid sequence set forth in any one of SEQ ID NOs: 1-299, as recited in column A of Table 1) can be modified at a base moiety, sugar moiety and/or phosphate backbone. Accordingly, RNA processing modulators (e.g., antisense oligonucleotides) may have one or more modified nucleotides (e.g., a nucleotide analog) and/or one or more backbone modifications (e.g., a modified internucleotide linkage). RNA processing modulators (e.g., antisense oligonucleotides) may have a combination of modified and unmodified nucleotides. RNA processing modulators (e.g., antisense oligonucleotides) may also have a combination of modified and unmodified internucleotide linkages. RNA processing modulators (e.g., antisense oligonucleotides) may comprise one or more chemical modifications (or combinations of chemical modifications) from Column C of Table 1. In some embodiments, an RNA processing modulator comprises a nucleic acid sequence from Column A of Table 1 and one or more chemical modifications (or combinations of chemical modifications) from Column C of Table 1, where Columns A and C are from the same row of Table 1.

In some embodiments, the one or more modifications is between 1 and 50 modifications, 2 and 20, 5 and 30, 10 and 40, or 15 and 50 modifications. In some embodiments, an RNA processing modulator (e.g., antisense oligonucleotide) comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications. In some embodiments, an RNA processing modulator (e.g., antisense oligonucleotide) comprises more than 50 modifications (e.g., 60, 70, 80, 90, 100, etc., modifications). In some embodiments, an RNA processing modulator (e.g., antisense oligonucleotide) comprises no more than 100 modifications. In some embodiments, an RNA processing modulator (e.g., antisense oligonucleotide) comprises chemical modifications on each nucleotide and each sugar-phosphate backbone linkage. Such a modified RNA processing modulator (e.g., antisense oligonucleotide) may be referred to as a “fully modified” RNA processing modulator (e.g., antisense oligonucleotide). In some embodiments, a fully modified antisense oligonucleotide comprises the nucleic acid sequence of any one of SEQ ID NOs: 1-303. In some embodiments, not all of the nucleotides of an antisense oligonucleotide are modified.

RNA processing modulators (e.g., antisense oligonucleotides) may include ribonucleotides, deoxyribonucleotides, and combinations thereof (e.g., RNA processing modulators comprising a gapmer structure). Examples of modified nucleotides which can be used in antisense nucleic acids include, for example, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, and 2,6-diaminopurine.

In some embodiments, a modified nucleotide is a 2′-modified nucleotide. For example, the 2′-modified nucleotide may be a 2′-deoxy, 2′-fluoro, 2′-O-methyl, 2′-O-methoxyethyl, 2′-amino and 2′-aminoalkoxy modified nucleotide. In some embodiments, the 2′-modified nucleotide comprises a 2′-O-4′-C methylene bridge, such as a locked nucleic acid (LNA) nucleotide. In some embodiments of a 2′ modified nucleotide the 2′-hydroxyl group is linked to the 3′ or 4′ carbon atom of the sugar ring thereby forming a bicyclic sugar moiety. In such embodiments, the linkage may be a methylene (~CH2-)ₙ group bridging the 2′ oxygen atom and the 3′ or 4′ carbon atom wherein n is 1 or 2. In some embodiments, a linkage comprises a cEt modification (e.g., a —CH3 replacing a hydrogen in the methylene group of the bridge).

RNA processing modulators (e.g., antisense oligonucleotides) may include combinations of LNA nucleotides and unmodified nucleotides. Antisense nucleic acids may include combinations LNA and RNA nucleotides. Antisense nucleic acids may include combinations LNA and DNA nucleotides. A further preferred oligonucleotide modification includes Locked Nucleic Acids (LNAs) in which the 2′-hydroxyl group is linked to the 3′ or 4′ carbon atom of the sugar ring thereby forming a bicyclic sugar moiety.

RNA processing modulators (e.g., antisense oligonucleotides) acids may also include nucleobase-modified nucleotides, e.g., nucleotides containing a non-naturally occurring nucleobase instead of a naturally occurring nucleobase. Bases may be modified to block the activity of adenosine deaminase, for example. Examples of modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

Within antisense nucleic acids (e.g., antisense oligonucleotides) of the disclosure, as few as one and as many as all nucleotides can be modified. In some embodiments, a modified RNA processing modulator (e.g., antisense oligonucleotide) will contain as few modified nucleotides as are necessary to achieve a desired level of in vivo stability and/or bioaccessibility or other desired property.

Certain antisense oligonucleotides may include non-ionic DNA analogs, such as alkyl- and aryl-phosphonates (in which the charged non-bridging oxygen is replaced by an alkyl or aryl group), phosphodiester and alkylphosphotriesters, in which the charged oxygen moiety is alkylated. Nucleic acids which contain a diol, such as tetraethyleneglycol or hexaethyleneglycol, at either or both termini have also been shown to be substantially resistant to nuclease degradation and may be used herein. In some embodiments, antisense nucleic acids may include at least one lipophilic substituted nucleotide analog and/or a pyrimidine-purine dinucleotide.

In some embodiments, RNA processing modulators (e.g., antisense oligonucleotides) may have one or two accessible 5′ ends. It is possible to create modified oligonucleotides having two such 5′ ends, for instance, by attaching two oligonucleotides through a 3′-3′ linkage to generate an oligonucleotide having one or two accessible 5′ ends. The 3′-3′-linkage may be a phosphodiester, phosphorothioate, or any other modified internucleoside bridge. Additionally, 3′-3′-linked oligonucleotides where the linkage between the 3′ terminal nucleosides is not a phosphodiester, phosphorothioate, or other modified bridge, can be prepared using an additional spacer, such as tri- or tetra-ethylenglycol phosphate moiety.

A phosphodiester internucleotide linkage of an RNA processing modulator (e.g., antisense oligonucleotide) can be replaced with a modified linkage. The modified linkage may be selected from, for example, phosphorothioate, phosphorodithioate, NR1R2-phosphoramidate, borano-phosphate, a-hydroxybenzyl phosphonate, phosphate-(C1-C21)-O-alkyl ester, phosphate-[(C6-C12)aryl-(C1-C21)-O-alkyl]ester, (C1-C8)alkylphosphonate and/or (C6-C12)arylphosphonate bridges, and (C7-C12)-α-hydroxymethyl-aryl. In some embodiments, a triazole ring is used.

A phosphate backbone of the RNA processing modulators (e.g., antisense oligonucleotides) can be modified to generate peptide nucleic acid molecules. As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols, for example.

RNA processing modulators (e.g., antisense oligonucleotides) also be formulated as morpholino oligonucleotides. In such embodiments, the riboside moiety of each subunit of an oligonucleotide of the oligonucleotide reagent is converted to a morpholine moiety. Morpholinos may also be modified, e.g., as peptide conjugated morpholino, etc.

Aspects of the disclosure relate to RNA processing modulators (e.g., antisense oligonucleotides) comprising a “gapmer” structure. A “gapmer” refers to an antisense oligonucleotide comprising the following formula Xₙ₁—(Y)ₙ₂—(X)ₙ₃, where (X) is a ribonucleotide (e.g., an RNA base) and (Y) is a deoxyribonucleotide (e.g., DNA base), and where each of n1, n2, and n3 are an integer ranging from 1 to 50 (inclusive of all integers therebetween). In some embodiments, antisense oligonucleotides having a gapmer structure bind (e.g., hybridize) to a target mRNA (e.g., an mRNA encoded by a GLS1 gene) and induce ribonuclease H1 (RNAseH1)-mediated degradation of the target mRNA. Gapmer antisense oligonucleotides are known in the art, for example as described by Kasuya et al. Sci Rep. 2016; 6: 30377.

The number of DNA bases in a gapmer may vary. In some embodiments, a gapmer comprises between 1 and 10 DNA bases (e.g., 1, 2, 3, 4, 5, 6, 7, 8, 9, or 10 DNA bases). In some embodiments, a gapmer comprises between 2 and 6 DNA bases (e.g., 2, 3, 4, 5, or 6 DNA bases). The DNA bases of a gapmer antisense oligonucleotide may be positioned toward to 5′ end of the ASO (e.g., within 1, 2, 3, 4, 5, etc. nucleotides of the 5′ terminal nucleotide of the ASO), toward the 3′ end of the ASO (e.g., within 1, 2, 3, 4, 5, etc. nucleotides of the 3′ terminal nucleotide of the ASO), or in the middle of the ASO (e.g., having an equal number of RNA bases flanking the DNA bases).

In other embodiments, an RNA processing modulator (e.g., antisense oligonucleotide) can be linked to functional groups, such as peptides (e.g., for targeting host cell receptors in vivo), or agents facilitating transport across the cell membrane or the blood-brain barrier. For example, oligonucleotide reagents of the disclosure also may be modified with chemical moieties (e.g., cholesterol) that improve the in vivo pharmacological properties of the RNA processing modulator. In some embodiments, a functional group comprises a peptide, small molecule, sugar, lipid, nucleic acid, or combination of any of the foregoing.

Sequences and chemical modifications of representative RNA processing modulators (e.g., antisense oligonucleotides) targeting GLS1 (e.g., an mRNA encoded by a GLS1 gene, such as a pre-mRNA or mature mRNA) are shown in Columns A and C, respectively, of Table 1.

TABLE 1
Representative RPMs targeting GLS1

| Seq. | Column A | Column B | Column C |
|---|---|---|---|
| No. | Sequence (5′ to 3′) | Region | Chemistry |
| | | | *Gapmers are indicated by structures |
| | | | denoted (X)-(Y)-(X), wherein “(X)” refers to |
| | | | regions comprising an ‘X’ number of |
| | | | nucleotide positions having ribose and which |
| | | | flank a region referred to as “(Y)” |
| | | | comprising a ‘Y’ number of nucleotide |
| | | | positions having deoxyribose |
| | | | **Skipmers (alternatively referred to herein |
| | | | as or exon-skippers skippers) are indicated |
| | | | by the lack of notation reciting “(X)-(Y)- |
| | | | (X)” and each nucleotide position comprises |
| | | | ribose |
| | | | Abbreviations: |
| | | | “Full PS” refers to each nucleotide position |
| | | | linked by an internucleotide linkage |
| | | | comprising a phosphorothioate modification |
| | | | unless otherwise indicated by nucleotide |
| | | | positions comprising a phosphodiester group |
| | | | (PO) at the 3′ carbon of ribose |
| | | | “2′MOE” refers to the 2′ carbon of ribose |
| | | | bonded to an oxygen atom which is bonded |
| | | | to a methoxyethyl group |
| | | | “#mer” refers to the number of nucleotide |
| | | | positions |
| | | | “PO” refers to a nucleotide position, |
| | | | wherein the 3′ carbon of ribose is linked |
| | | | through a phosphodiester bond which links |
| | | | the nucleotide position at its 5′ carbon to an |
| | | | immediately adjacent nucleotide position in |
| | | | the 3′ direction |
| 1 | CACTCTTTCAACCTGGGA | Exon_3 | Full PS; 2′MOE; 18mer |
| 2 | CTAAGGCTCCGCACTGAGGC | Exon-1 + Intron | Full PS; 2′MOE; 20mer |
| 3 | ATAGAACAGCAAATCTTC | Exon_2 | Full PS; 2′MOE; 18mer |
| 4 | GGCAGAAACTTTTACTTT | Intron+Exon-3 | Full PS; 2′MOE; 18mer |
| 5 | AAGATCATTAACAGCAAT | | Full PS; 2′MOE; 18mer; 4-10-4, PO after |
| | | | 2nd from 5′ end, PO after 3rd position from |
| | | | 3′ end |
| 6 | ACACCATCTGATGTTGTT | | Full PS; 2′MOE; 18mer |
| 7 | TATTCATTACCAGCCATC | Exon_9 | Full PS; 2′MOE; 18mer |
| 8 | CTTGTGTCAACAAAACAA | | Full PS; 2′MOE; 18mer; 4-10-4, PO after |
| | | | 2nd from 5′ end, PO after 3rd position from |
| | | | 3′ end |
| 9 | CCTGAGAAGTCATACATG | Exon_12 | Full PS; 2′MOE; 18mer |
| 10 | AAGATCATTAACAGCAATGG | Exon | Full PS; 2′MOE; 20mer; 5-10-5 |
| 11 | CTCTTTCAACCTGGGATCAG | | Full PS; 2′MOE; 20mer |
| 12 | CTTACATAGTCAAATTTT | Exon_8 | Full PS; 2′MOE; 18mer |
| 13 | GAAAGGAACGAGGCCTCCTG | Intron | Full PS; 2′MOE; 20mer |
| 14 | CCACAAATCGGGACTGAATT | Exon-5 | Full PS; 2′MOE; 20mer |
| 15 | GTGTCAACAAAACAATGTTG | Exon | Full PS; 2′MOE; 20mer; 5-10-5 |
| 16 | CCATACACTCTTTCAACCTG | | Full PS; 2′MOE; 20mer |
| 17 | CTCGGACTCCCGCAGCGGAT | 5′ UTR | Full PS; 2′MOE; 20mer |
| 18 | ATTAACAGCAATGGCATA | | Full PS; 2′MOE; 18mer; 4-10-4, PO after |
| | | | 2nd from 5′ end, PO after 3rd position from |
| | | | 3′ end |
| 19 | CATACACTCTTTCAACCT | | Full PS; 2′MOE; 18mer |
| 20 | CAAGCTAGGTAACAGACC | Exon_2 | Full PS; 2′MOE; 18mer; 4-10-4 |
| 21 | GCAAAGTGTCTCAAATTA | Exon_14 | Full PS; 2′MOE; 18mer |
| 22 | TATAGAACAGCAAATCTT | | Full PS; 2′MOE; 18mer |
| 23 | TTTTCACCTGAGGCCACCAG | Exon-1 | Full PS; 2′MOE; 20mer |
| 24 | GCTAGGTAACAGACCCTG | Exon_2 | Full PS; 2′MOE; 18mer |
| 25 | CAAGCTAGGTAACAGACC | | Full PS; 2′MOE; 18mer |
| 26 | ATGTTGTTTGAAGAGTTA | Exon_3 | Full PS; 2′MOE; 18mer |
| 27 | CAACTTCAACATGACCTA | Exon_17_Alt | Full PS; 2′MOE; 18mer |
| 28 | CTCTTTGCCCTCGCTGTTGC | Exon-1 | Full PS; 2′MOE; 20mer |
| 29 | ATACACTCTTTCAACCTG | | Full PS; 2′MOE; 18mer |
| 30 | GATCATTAACAGCAATGG | | Full PS; 2′MOE; 18mer; 4-10-4, PO after |
| | | | 2nd from 5′ end, PO after 3rd position from |
| | | | 3′ end |
| 31 | TAACATATCCATACACTCTT | Exon-3 | Full PS; 2′MOE; 20mer |
| 32 | ACTTTTCACCTGAGGCCA | Exon_1 | Full PS; 2′MOE; 18mer |
| 33 | AAAGATCTTTGTCTAGCA | | Full PS; 2′MOE; 18mer; 4-10-4, PO after |
| | | | 2nd from 5′ end, PO after 3rd position from |
| | | | 3′ end |
| 34 | ATACACTCTTTCAACCTGGG | Exon-3 | Full PS; 2′MOE; 20mer |
| 35 | TCTGATGTTGTTTGAAGA | | Full PS; 2′MOE; 18mer |
| 36 | ATAGAACAGCAAATCTTCCA | Exon | Full PS; 2′MOE; 20mer; 5-10-5 |
| 37 | CCAGCACCCAAGATTGAGAA | ENST00000409626.5_ | Full PS; 2′MOE; 20mer; 5-10-5; PO after |
| | | utr3_3_0_ | 2nd from 5′ end, PO after 3rd position from |
| | | chr2_190932863_f | 3′ end |
| 38 | GTTGTTTGAAGAGTTAATCT | Exon-3 | Full PS; 2′MOE; 20mer |
| 39 | CACTCACTTTTCACCTGA | Exon_1 | Full PS; 2′MOE; 18mer |
| 40 | TTATGAACAGGTATTTTCTC | Exon-2 | Full PS; 2′MOE; 20mer |
| 41 | GTGTCAACAAAACAATGT | | Full PS; 2′MOE; 18mer; 4-10-4, PO after |
| | | | 2nd from 5′ end, PO after 3rd position from |
| | | | 3′ end |
| 42 | ATTAAAGGTTTGGCAGAA | Exon_3 | Full PS; 2′MOE; 18mer |
| 43 | ATCAATGTGTGAGGTAAA | Exon_4 | Full PS; 2′MOE; 18mer |
| 44 | CTAGGTAACAGACCCTGTTT | Exon-2 | Full PS; 2′MOE; 20mer |
| 45 | CAGCATTTACCATAGGAT | Exon_7 | Full PS; 2′MOE; 18mer; 4-10-4 |
| 46 | TGTCAACAAAACAATGTT | | Full PS; 2′MOE; 18mer; 4-10-4, PO after |
| | | | 2nd from 5′ end, PO after 3rd position from |
| | | | 3′ end |
| 47 | TACACTCTTTCAACCTGGGA | | Full PS; 2′MOE; 20mer |
| 48 | CATATCGATGCACATATTCA | Exon | Full PS; 2′MOE; 20mer; 5-10-5 |
| 49 | GACACCATCTGATGTTGTTT | Exon | Full PS; 2′MOE; 20mer; 5-10-5 |
| 50 | CAATTGTATAGAACAGCA | | Full PS; 2′MOE; 18mer |
| 51 | ACCTCAAATAAATATTTATA | ENST00000409626.5_ | Full PS; 2′MOE; 20mer; 5-10-5; PO after |
| | | utr3_3_0_chr2_ | 2nd from 5′ end, PO after 3rd position from |
| | | 190932863_f | 3′ end |
| 52 | CGCCTCGCGGAGACACTCAC | Intron | Full PS; 2′MOE; 20mer |
| 53 | GGGCAGAAACCACCATTA | Exon_12 | Full PS; 2′MOE; 18mer; 4-10-4 |
| 54 | GCTGCTGCTGCTGCTGCTGC | 5′ UTR | Full PS; 2′MOE; 20mer |
| 55 | CTCTTTCAACCTGGGATC | Exon_3 | Full PS; 2′MOE; 18mer; 4-10-4 |
| 56 | GTAATTGGGCAGAAACCA | Exon_12 | Full PS; 2′MOE; 18mer |
| 57 | GGCCAGTTGAGGAATATA | Exon_5 | Full PS; 2′MOE; 18mer |
| 58 | TACACTCTTTCAACCTGGGA | | Full PS; 2′MOE; 20mer; 5-10-5; PO after |
| | | | 2nd from 5′ end, PO after 3rd position from |
| | | | 3′ end |
| 59 | ACTCTTTCAACCTGGGAT | | Full PS; 2′MOE; 18mer |
| 60 | CTCACTTTTCACCTGAGGCC | Intron + Exon-1 | Full PS; 2′MOE; 20mer |
| 61 | CCATCTGATGTTGTTTGA | | Full PS; 2′MOE; 18mer |
| 62 | TTGGTCACCAAACATATC | Exon_18_Alt | Full PS; 2′MOE; 18mer; 4-10-4 |
| 63 | TTCTAAATGCTTGTGTCAAC | Exon-4 | Full PS; 2′MOE; 20mer |
| 64 | GCTGCTGCGCGCTAGGATTG | 5′ UTR | Full PS; 2′MOE; 20mer |
| 65 | CTCTGTCCATCTACTGTACA | Exon-5 | Full PS; 2′MOE; 20mer |
| 66 | TCTTTCAACCTGGGATCA | | Full PS; 2′MOE; 18mer |
| 67 | AGATCTTTGTCTAGCATG | Exon_3 | Full PS; 2′MOE; 18mer; 4-10-4 |
| 68 | AAGATCTTTGTCTAGCAT | | Full PS; 2′MOE; 18mer; 4-10-4, PO after |
| | | | 2nd from 5′ end, PO after 3rd position from |
| | | | 3′ end |
| 69 | CTACTTGGGTGTGGTTCCGG | 5′ UTR | Full PS; 2′MOE; 20mer |
| 70 | TGGACAGACTGTACAACA | Exon_18_Alt | Full PS; 2′MOE; 18mer |
| 71 | GATCTTTGTCTAGCATGA | | Full PS; 2′MOE; 18mer; 4-10-4, PO after |
| | | | 2nd from 5′ end, PO after 3rd position from |
| | | | 3′ end |
| 72 | TTTAAGTTAAGATAGAAA | Exon_2 | Full PS; 2′MOE; 18mer |
| 73 | TTTCATATAACTCATCAA | Exon_4 | Full PS; 2′MOE; 18mer |
| 74 | TGTATAGAACAGCAAATC | | Full PS; 2′MOE; 18mer |
| 75 | AACAGACACACCCCACAAAT | Exon-5 | Full PS; 2′MOE; 20mer |
| 76 | TTAAAAAGATCTTTGTCT | | Full PS; 2′MOE; 18mer; 4-10-4, PO after |
| | | | 2nd from 5′ end, PO after 3rd position from |
| | | | 3′ end |
| 77 | AGCATTATTTACTCCTTGCT | Exon | Full PS; 2′MOE; 20mer; 5-10-5 |
| 78 | CATATAACTCATCAATGTGT | Exon-4 | Full PS; 2′MOE; 20mer |
| 79 | TTGAAGAGTTAATCTTAA | Exon_3 | Full PS; 2′MOE; 18mer |
| 80 | CTGCTGCTGCTGCTGCTGCT | 5′ UTR | Full PS; 2′MOE; 20mer |
| 81 | TAAATGAAGAAACATTCTGA | ENST00000409626.5_ | Full PS; 2′MOE; 20mer; 5-10-5; PO after |
| | | utr3_3_0_ | 2nd from 5′ end, PO after 3rd position from |
| | | chr2_190932863_f | 3′ end |
| 82 | ATAGCATAGCAAATCACC | Exon 3 | Full PS; 2′MOE; 18mer |
| 83 | CATCATGCCGCCGGGTCCGT | Exon- | Full PS; 2′MOE; 20mer |
| | | 1 + AUG + 5′UTR | |
| 84 | AAGACATAAAGTCAGGAA | Exon_4 | Full PS; 2′MOE; 18mer; 4-10-4 |
| 85 | AGACACTCACTTTTCACCTG | Intron + Exon-1 | Full PS; 2′MOE; 20mer |
| 86 | AGATCTTTGTCTAGCATGAC | Exon-3 | Full PS; 2′MOE; 20mer |
| 87 | CGCGGAGACACTCACTTTTC | Intron + Exon-1 | Full PS; 2′MOE; 20mer |
| 88 | TTGTCCTTCAGCAATTGTAT | Exon-2 | Full PS; 2′MOE; 20mer |
| 89 | TGTTGAATCTTAGTCCACTC | Exon | Full PS; 2′MOE; 20mer; 5-10-5 |
| 90 | GCAAATCACCATTAAAGG | Exon_3 | Full PS; 2′MOE; 18mer |
| 91 | CCCTGAGAAGTCATACAT | Exon_12 | Full PS; 2′MOE; 18mer; 4-10-4 |
| 92 | GGAGACACTCACTTTTCA | Exon_1 | Full PS; 2′MOE; 18mer |
| 93 | ACACTCTTTCAACCTGGGAT | | Full PS; 2′MOE; 20mer |
| 94 | GAGACACTCACTTTTCAC | Exon_1 | Full PS; 2′MOE; 18mer |
| 95 | GTATAGCATAGCAAATCA | Exon_3 | Full PS; 2′MOE; 18mer |
| 96 | GTGGTTCCGGCTCGGACTCC | 5′ UTR | Full PS; 2′MOE; 20mer |
| 97 | CATACACTCTTTCAACCTGG | | Full PS; 2′MOE; 20mer; 5-10-5; PO after |
| | | | 2nd from 5′ end, PO after 3rd position from |
| | | | 3′ end |
| 98 | ATCTTTGTCTAGCATGAC | | Full PS; 2′MOE; 18mer; 4-10-4, PO after |
| | | | 2nd from 5′ end, PO after 3rd position from |
| | | | 3′ end |
| 99 | CATACACTCTTTCAACCTGG | | Full PS; 2′MOE; 20mer |
| 100 | GTCACCAAACATATCTTA | Exon_18_Alt | Full PS; 2′MOE; 18mer; 4-10-4 |
| 101 | TGGCCAGTTGAGGAATATAA | Exon-5 | Full PS; 2′MOE; 20mer |
| 102 | CCATACACTCTTTCAACCTG | | Full PS; 2′MOE; 20mer; 5-10-5; PO after |
| | | | 2nd from 5′ end, PO after 3rd position from |
| | | | 3′ end |
| 103 | CCAAGATCATTAACAGCA | | Full PS; 2′MOE; 18mer; 4-10-4, PO after |
| | | | 2nd from 5′ end, PO after 3rd position from |
| | | | 3′ end |
| 104 | CTGATGTTGTTTGAAGAG | | Full PS; 2′MOE; 18mer |
| 105 | TTATGTGGTTTATCTGTA | Exon_7 | Full PS; 2′MOE; 18mer; 4-10-4 |
| 106 | CCAACATATTCATTACCA | Exon_9 | Full PS; 2′MOE; 18mer |
| 107 | GATTATGTGGTTTATCTG | Exon_7 | Full PS; 2′MOE; 18mer |
| 108 | CTTACTGTAATAAATTTATG | Intron + Exon-2 | Full PS; 2′MOE; 20mer |
| 109 | AGTGTCTCAAATTATCAT | Exon_14 | Full PS; 2′MOE; 18mer; 4-10-4 |
| 110 | CACTCTTTCAACCTGGGATC | | Full PS; 2′MOE; 20mer |
| 111 | TTGGGCAGAAACCACCAT | Exon_12 | Full PS; 2′MOE; 18mer |
| 112 | ATAAACTTACCTCTGTCCAT | Intron + Exon-5 | Full PS; 2′MOE; 20mer |
| 113 | GCTAGTTGTTCAAGCAAC | Exon_15_Alt | Full PS; 2′MOE; 18mer |
| 114 | TGATTCACAAGTCACTTCAA | Exon | Full PS; 2′MOE; 20mer; 5-10-5 |
| 115 | AAAGATCTTTGTCTAGCATG | Exon | Full PS; 2′MOE; 20mer; 5-10-5 |
| 116 | ACCATCTGATGTTGTTTG | | Full PS; 2′MOE; 18mer |
| 117 | AAAGCATTACCTTTCCTCCA | Intron + Exon-4 | Full PS; 2′MOE; 20mer |
| 118 | GCAATTGTATAGAACAGC | Exon_2 | Full PS; 2′MOE; 18mer |
| 119 | TAGTGAAGTCACAACAATTG | Exon | Full PS; 2′MOE; 20mer; 5-10-5 |
| 120 | CCGCGACCGGTTCTCTTCGC | 5′ UTR | Full PS; 2′MOE; 20mer |
| 121 | ATTACCTTTCCTCCAGACTG | Intron + Exon-4 | Full PS; 2′MOE; 20mer |
| 122 | GTAACAGACCCTGTTTTATT | Exon | Full PS; 2′MOE; 20mer; 5-10-5 |
| 123 | CCAAGCTAGGTAACAGAC | Exon_2 | Full PS; 2′MOE; 18mer |
| 124 | AGTTAATCTTAACATATC | Exon_3 | Full PS; 2′MOE; 18mer |
| 125 | ATCACCATTAAAGGTTTG | Exon_3 | Full PS; 2′MOE; 18mer |
| 126 | TACTGTAATAAATTTATG | Exon_2 | Full PS; 2′MOE; 18mer |
| 127 | ACACTCTTTCAACCTGGGAT | | Full PS; 2′MOE; 20mer; 5-10-5; PO after |
| | | | 2nd from 5′ end, PO after 3rd position from |
| | | | 3′ end |
| 128 | TTGTGTCAACAAAACAAT | | Full PS; 2′MOE; 18mer; 4-10-4, PO after |
| | | | 2nd from 5′ end, PO after 3rd position from |
| | | | 3′ end |
| 129 | CCATACACTCTTTCAACC | Exon_3 | Full PS; 2′MOE; 18mer |
| 130 | ACAAACTTTCTTCTAAATGC | Exon-4 | Full PS; 2′MOE; 20mer |
| 131 | TTTAGCACTTTCATATAA | Exon_4 | Full PS; 2′MOE; 18mer |
| 132 | TAGCATGACACCATCTGA | Exon_3 | Full PS; 2′MOE; 18mer |
| 133 | AGACATAAAGTCAGGAAT | Exon_4 | Full PS; 2′MOE; 18mer |
| 134 | TAGATTTGAGTGCCTGTTTT | Exon-3 + Intron | Full PS; 2′MOE; 20mer |
| 135 | GACACCATCTGATGTTGT | | Full PS; 2′MOE; 18mer |
| 136 | GTCCCGCAGCATCCCCGAGC | Exon-1 | Full PS; 2′MOE; 20mer |
| 137 | TAGGATTATGTGGTTTAT | Exon_7 | Full PS; 2′MOE; 18mer; 4-10-4 |
| 138 | ATCATTAACAGCAATGGC | | Full PS; 2′MOE; 18mer; 4-10-4, PO after |
| | | | 2nd from 5′ end, PO after 3rd position from |
| | | | 3′ end |
| 139 | CTTTCAACCTGGGATCAG | | Full PS; 2′MOE; 18mer |
| 140 | GTCAACAAAACAATGTTG | | Full PS; 2′MOE; 18mer; 4-10-4, PO after |
| | | | 2nd from 5′ end, PO after 3rd position from |
| | | | 3′ end |
| 141 | CACCATCTGATGTTGTTT | | Full PS; 2′MOE; 18mer |
| 142 | TGCTGCTGCTGCTGCTGCGC | 5′ UTR | Full PS; 2′MOE; 20mer |
| 143 | GCAATCCTGTAGATTTGAGT | Exon | Full PS; 2′MOE; 20mer; 5-10-5 |
| 144 | GCACTTTCATATAACTCA | Exon_4 | Full PS; 2′MOE; 18mer |
| 145 | ATCCCCGAGCCTCGCAGCCG | Exon-1 | Full PS; 2′MOE; 20mer |
| 146 | GCCCTGAGAAGTCATACA | Exon_12 | Full PS; 2′MOE; 18mer |
| 147 | AGATCTTTGTCTAGCATG | | Full PS; 2′MOE; 18mer; 4-10-4, PO after |
| | | | 2nd from 5′ end, PO after 3rd position from |
| | | | 3′ end |
| 148 | CTTACCTCTGTCCATCTACT | Intron + Exon-5 | Full PS; 2′MOE; 20mer |
| 149 | ACACTCTTTCAACCTGGG | | Full PS; 2′MOE; 18mer |
| 150 | ATTGAGAACAAAAAATACCT | ENST00000409626.5_ | Full PS; 2′MOE; 20mer; 5-10-5; PO after |
| | | utr3_3_0_chr2_ | 2nd from 5′ end, PO after 3rd position from |
| | | 190932863_f | 3′ end |
| 151 | TATAGAACAGCAAATCTT | Exon_2 | Full PS; 2′MOE; 18mer; 4-10-4 |
| 152 | CAAATCACCATTAAAGGT | Exon_3 | Full PS; 2′MOE; 18mer; 4-10-4 |
| 153 | TTACCATCTTCATTCAAA | Exon_6 | Full PS; 2′MOE; 18mer |
| 154 | CGCAGCGGATGCGGGTGCTG | 5′ UTR | Full PS; 2′MOE; 20mer |
| 155 | CCAGCATTTACCATAGGA | Exon_7 | Full PS; 2′MOE; 18mer |
| 156 | GGGAAAGGGTTTACTTTG | Exon_17_Alt | Full PS; 2′MOE; 18mer |
| 157 | CAAGATCATTAACAGCAA | | Full PS; 2′MOE; 18mer; 4-10-4, PO after |
| | | | 2nd from 5′ end, PO after 3rd position from |
| | | | 3′ end |
| 158 | TGTAATAAATTTATGAACAG | Exon-2 | Full PS; 2′MOE; 20mer |
| 159 | AGAAACCACCATTAGCCA | Exon_12 | Full PS; 2′MOE; 18mer |
| 160 | ATCAATGTGTGAGGTAAAAG | Exon-4 | Full PS; 2′MOE; 20mer |
| 161 | GCGGTGAGATGACAGAAGAG | 5′ UTR | Full PS; 2′MOE; 20mer |
| 162 | CTTACCATCTTCATTCAA | Exon_6 | Full PS; 2′MOE; 18mer |
| 163 | GAACGCGGTCTGTGGTGG | 5′ UTR | Full PS; 2′MOE; 18mer |
| 164 | TGAAGAAACATTCTGAAACT | ENST00000409626.5_ | Full PS; 2′MOE; 20mer; 5-10-5; PO after |
| | | utr3_3_0_chr2_ | 2nd from 5′ end, PO after 3rd position from |
| | | 190932863_f | 3′ end |
| 165 | CCAGCCATCTTATTCAAA | Exon_9 | Full PS; 2′MOE; 18mer |
| 166 | GTGTCAACAAAACAATGTTG | | Full PS; 2′MOE; 20mer; Gapmer, 5-10-5, PO |
| | | | after 2nd from 5′ end, PO after 3rd position |
| | | | from 3′ end |
| 167 | GAGTTAATCTTAACATATCC | Exon-3 | Full PS; 2′MOE; 20mer |
| 168 | TCAACCTGGGATCAGACGTT | Exon-3 | Full PS; 2′MOE; 20mer |
| 169 | TTTACACAGGACTGAAGA | Exon_6 | Full PS; 2′MOE; 18mer |
| 170 | GACAGAAGAGGAAAGGGCAG | 5′ UTR | Full PS; 2′MOE; 20mer |
| 171 | GTTGAGGAATATAATCTG | Exon_5 | Full PS; 2′MOE; 18mer |
| 172 | AAGACATAAAGTCAGGAATC | Exon-4 | Full PS; 2′MOE; 20mer |
| 173 | TTCACCTGAGGCCACCAG | Exon_1 | Full PS; 2′MOE; 18mer |
| 174 | GCACTTACATAGTCAAAT | Exon_8 | Full PS; 2′MOE; 18mer |
| 175 | TTTCAACCTGGGATCAGA | Exon_3 | Full PS; 2′MOE; 18mer |
| 176 | TAACTCATCAATGTGTGA | Exon_4 | Full PS; 2′MOE; 18mer |
| 177 | CTCTTTCAACCTGGGATCAG | | Full PS; 2′MOE; 20mer; 5-10-5; PO after |
| | | | 2nd from 5′ end, PO after 3rd position from |
| | | | 3′ end |
| 178 | AGGCCTCCTGCGCCTCGCGG | Intron | Full PS; 2′MOE; 20mer |
| 179 | GCGGGTGCTGCTGCTGCTGC | 5′ UTR | Full PS; 2′MOE; 20mer |
| 180 | CAAAAAATACCTCAAATAAA | ENST00000409626.5_ | Full PS; 2′MOE; 20mer; 5-10-5; PO after |
| | | utr3_3_0_chr2_ | 2nd from 5′ end, PO after 3rd position from |
| | | 190932863_f | 3′ end |
| 181 | AGATCATTAACAGCAATG | | Full PS; 2′MOE; 18mer; 4-10-4, PO after |
| | | | 2nd from 5′ end, PO after 3rd position from |
| | | | 3′ end |
| 182 | TTCTCTTCGCTCCGCCTAAG | 5′ UTR | Full PS; 2′MOE; 20mer |
| 183 | TTGGAAAGAAATGAATAG | Exon_5 | Full PS; 2′MOE; 18mer |
| 184 | AACAATGTTGCTCTGAACAC | Exon-4 | Full PS; 2′MOE; 20mer |
| 185 | TACACTCTTTCAACCTGG | | Full PS; 2′MOE; 18mer |
| 186 | AGGAATATAATCTGCAACCT | Exon-5 + Intron | Full PS; 2′MOE; 20mer |
| 187 | CTACTGTACAAACAGACACA | Exon-5 | Full PS; 2′MOE; 20mer |
| 188 | TACCATAGGATTATGTGGTT | Exon | Full PS; 2′MOE; 20mer; 5-10-5 |
| 189 | TGTTTGAAGAGTTAATCT | Exon_3 | Full PS; 2′MOE; 18mer |
| 190 | AACTTACTGTAATAAATT | Exon_2 | Full PS; 2′MOE; 18mer |
| 191 | TGTGTCAACAAAACAATG | | Full PS; 2′MOE; 18mer; 4-10-4, PO after |
| | | | 2nd from 5′ end, PO after 3rd position from |
| | | | 3′ end |
| 192 | AATTGTATAGAACAGCAA | | Full PS; 2′MOE; 18mer |
| 193 | TTGTATAGAACAGCAAAT | Exon_2 | Full PS; 2′MOE; 18mer |
| 194 | ACAACTTCAACATGACCT | Exon_17_Alt | Full PS; 2′MOE; 18mer; 4-10-4 |
| 195 | AATTATCATAGTTATGGA | Exon_14 | Full PS; 2′MOE; 18mer |
| 196 | GAGAAGTCATACATGCCACA | Exon | Full PS; 2′MOE; 20mer; 5-10-5 |
| 197 | TGATGTTGTTTGAAGAGT | | Full PS; 2′MOE; 18mer |
| 198 | GCTTACCCTTTGATCACC | Exon_14 | Full PS; 2′MOE; 18mer |
| 199 | GTATTTTCTCTTGTCCTTCA | Exon-2 | Full PS; 2′MOE; 20mer |
| 200 | GCAATTGTATAGAACAGCAA | Exon-2 | Full PS; 2′MOE; 20mer |
| 201 | TCATTACCAGCCATCTTA | Exon_9 | Full PS; 2′MOE; 18mer; 4-10-4 |
| 202 | TACCATAGGATTATGTGGTT | Exon | Full PS; 2′MOE; 20mer; 5-10-5 |
| 203 | ACTAGAACCCATTTCTTTAG | ENST00000409626.5_ | Full PS; 2′MOE; 20mer; 5-10-5; PO after |
| | | utr3_3_0_chr2_ | 2nd from 5′ end, PO after 3rd position from |
| | | 190932863_f | 3′ end |
| 204 | ATGTGGTTTATCTGTAAA | Exon_7 | Full PS; 2′MOE; 18mer |
| 205 | TGAACACATCTGTAGAATGT | Exon-4 + Intron | Full PS; 2′MOE; 20mer |
| 206 | CCGGGCGTGGGCGGCCGGTT | 5′ UTR | Full PS; 2′MOE; 20mer |
| 207 | ATCAGACGTTCGCAATCCTG | Exon-3 | Full PS; 2′MOE; 20mer |
| 208 | TTACCATCTTCATTCAAA | Exon_6 | Full PS; 2′MOE; 18mer; 4-10-4 |
| 209 | TTAGCACTTTCATATAACTC | Exon-4 | Full PS; 2′MOE; 20mer |
| 210 | AGGCCACCAGCTCTTTGCCC | Exon-1 | Full PS; 2′MOE; 20mer |
| 211 | ATCTTTGTCTAGCATGAC | Exon_3 | Full PS; 2′MOE; 18mer |
| 212 | TCATTAACAGCAATGGCA | | Full PS; 2′MOE; 18mer; 4-10-4, PO after |
| | | | 2nd from 5′ end, PO after 3rd position from |
| | | | 3′ end |
| 213 | AGAACAGCAAATCTTCCAAG | Exon-2 | Full PS; 2′MOE; 20mer |
| 214 | ACTCTTTCAACCTGGGATCA | | Full PS; 2′MOE; 20mer; 5-10-5; PO after |
| | | | 2nd from 5′ end, PO after 3rd position from |
| | | | 3′ end |
| 215 | CCCGGGCCCCGAAAGGAACG | Intron | Full PS; 2′MOE; 20mer |
| 216 | AAATTATCATAGTTATGG | Exon_14 | Full PS; 2′MOE; 18mer; 4-10-4 |
| 217 | ACACAGAGAAACAAGATC | Exon_14 | Full PS; 2′MOE; 18mer |
| 218 | CATCTGATGTTGTTTGAA | | Full PS; 2′MOE; 18mer |
| 219 | ATTGTATAGAACAGCAAA | Exon_2 | Full PS; 2′MOE; 18mer; 4-10-4 |
| 220 | TGACTCAGGTGACACTTT | Exon_15_Alt | Full PS; 2′MOE; 18mer |
| 221 | ATCTTCCAAGCTAGGTAACA | Exon-2 | Full PS; 2′MOE; 20mer |
| 222 | CATTTACCATAGGATTAT | Exon_7 | Full PS; 2′MOE; 18mer; 4-10-4 |
| 223 | TTTCACCAGTAATTGGGC | Exon_12 | Full PS; 2′MOE; 18mer |
| 224 | ACCATCTGATGTTGTTTGAA | Exon-3 | Full PS; 2′MOE; 20mer |
| 225 | ACATTCTGAAACTAGAACCC | ENST00000409626.5_ | Full PS; 2′MOE; 20mer; 5-10-5; PO after |
| | | utr3_3_0_chr2_ | 2nd from 5′ end, PO after 3rd position from |
| | | 190932863_f | 3′ end |
| 226 | GTCAGGAATCACAAACTTTC | Exon-4 | Full PS; 2′MOE; 20mer |
| 227 | CTAGCATGACACCATCTGAT | Exon-3 | Full PS; 2′MOE; 20mer |
| 228 | TTGACTCAGGTGACACTT | Exon_15_Alt | Full PS; 2′MOE; 18mer; 4-10-4 |
| 229 | GGACTGAATTTGGCCAGTTG | Exon-5 | Full PS; 2′MOE; 20mer |
| 230 | ATCTGATGTTGTTTGAAG | | Full PS; 2′MOE; 18mer |
| 231 | CTCTTTCAACCTGGGATC | | Full PS; 2′MOE; 18mer |
| 232 | CAAACGCGTCCGTCTCCCCG | Exon-1 | Full PS; 2′MOE; 20mer |
| 233 | ATACACTCTTTCAACCTGGG | | Full PS; 2′MOE; 20mer; 5-10-5; Gapmer, PO |
| | | | after 2nd from 5′ end, PO after 3rd position |
| | | | from 3′ end |
| 234 | TTGAAAGATTAAATGAAGAA | ENST00000409626.5_ | Full PS; 2′MOE; 20mer; 5-10-5; PO after |
| | | utr3_3_0_chr2_ | 2nd from 5′ end, PO after 3rd position from |
| | | 190932863_f | 3′ end |
| 235 | TCCGCCTAAGGCTCCGCACT | 5' UTR | Full PS; 2′MOE; 20mer |
| 236 | AAAGATCTTTGTCTAGCATG | | Full PS; 2′MOE; 20mer; Gapmer, 5-10-5, PO |
| | | | after 2nd from 5′ end, PO after 3rd position |
| | | | from 3′ end |
| 237 | CATTAACAGCAATGGCAT | | Full PS; 2′MOE; 18mer; 4-10-4, PO after |
| | | | 2nd from 5′ end, PO after 3rd position from |
| | | | 3′ end |
| 238 | TATACTACAGTTGTAGAG | Exon_15_Alt | Full PS; 2′MOE; 18mer |
| 239 | GCATAGCAAATCACCATT | Exon_3 | Full PS; 2′MOE; 18mer |
| 240 | CATACACTCTTTCAACCT | Exon_3 | Full PS; 2′MOE; 18mer; 4-10-4 |
| 241 | TGACACCATCTGATGTTG | | Full PS; 2′MOE; 18mer |
| 242 | AAGATCATTAACAGCAATGG | | Full PS; 2′MOE; 20mer; Gapmer, 5-10-5, PO |
| | | | after 2nd from 5′ end, PO after 3rd position |
| | | | from 3′ end |
| 243 | CACTCTTTCAACCTGGGATC | | Full PS; 2′MOE; 20mer; 5-10-5; PO after |
| | | | 2nd from 5′ end, PO after 3rd position from |
| | | | 3′ end |
| 244 | GGAGGATGCTCCGGGCGTGG | 5′ UTR | Full PS; 2′MOE; 20mer |
| 245 | TTACCATAGGATTATGTG | Exon_7 | Full PS; 2′MOE; 18mer |
| 246 | CTCGCAGCCGCATCATGCCG | Exon- | Full PS; 2′MOE; 20mer |
| | | 1 + AUG + 5′UTR | |
| 247 | TGAAGAGTTAATCTTAAC | Exon_3 | Full PS; 2′MOE; 18mer |
| 248 | AGAACCCATTTCTTTAGCTT | ENST00000409626.5_ | Full PS; 2′MOE; 20mer; 5-10-5; PO after |
| | | utr3_3_0_chr2_ | 2nd from 5′ end, PO after 3rd position from |
| | | 190932863_f | 3′ end |
| 249 | ATCTTATCCAGAGGAGGAGA | Exon | Full PS; 2′MOE; 20mer; 5-10-5 |
| 250 | CGCAATCCTGTAGATTTGAG | Exon-3 | Full PS; 2′MOE; 20mer |
| 251 | GAAAGGGCAGCTACTTGGGT | 5′ UTR | Full PS; 2′MOE; 20mer |
| 252 | TCGCTGTTGCCAAACGCGTC | Exon-1 | Full PS; 2′MOE; 20mer |
| 253 | GTTTTATTTTACTAAAGAGG | Exon-2 + Intron | Full PS; 2′MOE; 20mer |
| 254 | GTATAGAACAGCAAATCT | | Full PS; 2′MOE; 18mer |
| 255 | GGCAGAAACTTTTACTTT | Exon_3 | Full PS; 2′MOE; 18mer |
| 256 | ATTTGAAATAAACTTACC | Exon_5 | Full PS; 2′MOE; 18mer |
| 257 | ATTACCAGCCATCTTATT | Exon_9 | Full PS; 2′MOE; 18mer |
| 258 | ATTGTATAGAACAGCAAA | | Full PS; 2′MOE; 18mer |
| 259 | TTGTTCAAGCAACATACA | Exon_15_Alt | Full PS; 2′MOE; 18mer |
| 260 | GCTTGTGTCAACAAAACA | | Full PS; 2′MOE; 18mer; 4-10-4, PO after |
| | | | 2nd from 5′ end, PO after 3rd position from |
| | | | 3′ end |
| 261 | GCAATCCTGTAGATTTGAGT | | Full PS; 2′MOE; 20mer; Gapmer, 5-10-5, PO |
| | | | after 2nd from 5′ end, PO after 3rd position |
| | | | from 3′ end |
| 262 | GCTAGGATTGCCGCGACCGG | 5′ UTR | Full PS; 2′MOE; 20mer |
| 263 | CATATAACTCATCAATGT | Exon_4 | Full PS; 2′MOE; 18mer |
| 264 | ACTCTTTCAACCTGGGATCA | | Full PS; 2′MOE; 20mer |
| 265 | AAAGTTAAGGCTTAGAGT | Exon_5 | Full PS; 2′MOE; 18mer |
| 266 | TTCATTACCAGCCATCTTAT | Exon | Full PS; 2′MOE; 20mer; 5-10-5 |
| 267 | TGTAATAAATTTATGAAC | Exon_2 | Full PS; 2′MOE; 18mer |
| 268 | TAGCACTTTCATATAACT | Exon_4 | Full PS; 2′MOE; 18mer; 4-10-4 |
| 269 | AACAATTGCTCCAGCATTTA | Exon | Full PS; 2'MOE; 20mer; 5-10-5 |
| 270 | CTGAGGCCACCAGCTCTTTG | ENST00000320717.8_ | 20mer; Gapmer; 5-10-5, PO after 2nd base |
| | | exon_0_0_chr2_ | from 5′ end, PO after 3rd base from 3′ end; |
| | | 190880821_f | Full PS; 2′MOE |
| 271 | AGCTAGGTAACAGACCCTGT | ENST0000032017.8_ | 20mer; Gapmer; 5-10-5, PO after 2nd base |
| | | 7exon_ 10_chr2_ | from 5′ end, PO after 3rd base from 3′ end; |
| | | 190895152_f | Full PS; 2′MOE |
| 272 | GTATAGAACAGCAAATCTTC | ENST00000320717.8_ | 20mer; Gapmer; 5-10-5, PO after 2nd base |
| | | exon_10_chr2_ | from 5′ end, PO after 3rd base from 3′ end; |
| | | 190895152_f | Full PS; 2′MOE |
| 273 | GCAATTGTATAGAACAGCAA | ENST00000320717.8_ | 20mer; Gapmer; 5-10-5, PO after 2nd base |
| | | exon_10_chr2_ | from 5′ end, PO after 3rd base from 3′ end; |
| | | 190895152_f | Full PS; 2′MOE |
| 274 | TCTAGCATGACACCATCTGA | ENST00000320717.8_ | 20mer; Gapmer; 5-10-5, PO after 2nd base |
| | | exon_20_chr2_ | from 5′ end, PO after 3rd base from 3′ end; |
| | | 190895604_f | Full PS; 2′MOE |
| 275 | AGATCTTTGTCTAGCATGAC | ENST00000320717.8_ | 20mer; Gapmer; 5-10-5, PO after 2nd base |
| | | exon_2_0_chr2_ | from 5′ end, PO after 3rd base from 3′ end; |
| | | 190895604_f | Full PS; 2′MOE |
| 276 | GCTTGTGTCAACAAAACAAT | ENST00000320717.8_ | 20mer; Gapmer; 5-10-5, PO after 2nd base |
| | | exon_30_chr2_ | from 5′ end, PO after 3rd base from 3′ end; |
| | | 190900564_f | Full PS; 2′MOE |
| 277 | ATGTGTGAGGTAAAAGACAT | ENST00000320717.8_ | 20mer; Gapmer; 5-10-5, PO after 2nd base |
| | | exon_30_chr2_ | from 5′ end, PO after 3rd base from 3′ end; |
| | | 190900564_f | Full PS; 2′MOE |
| 278 | GGCCAGTTGAGGAATATAAT | ENST00000320717.8_ | 20mer; Gapmer; 5-10-5, PO after 2nd base |
| | | exon_40_chr2_ | from 5′ end, PO after 3rd base from 3′ end; |
| | | 190901947_f | Full PS; 2′MOE |
| 279 | TGAATTTGGCCAGTTGAGGA | ENST00000320717.8_ | 20mer; Gapmer; 5-10-5, PO after 2nd base |
| | | exon_4_0_chr2_ | from 5′ end, PO after 3rd base from 3′ end; |
| | | 190901947_f | Full PS; 2′MOE |
| 280 | ACTGTACAAACAGACACACC | ENST00000320717.8_ | 20mer; Gapmer; 5-10-5, PO after 2nd base |
| | | exon_40_chr2_ | from 5′ end, PO after 3rd base from 3′ end; |
| | | 190901947_f | Full PS; 2′MOE |
| 281 | CCATCTACTGTACAAACAGA | ENST00000320717.8_ | 20mer; Gapmer; 5-10-5, PO after 2nd base |
| | | exon_40_chr2_ | from 5′ end, PO after 3rd base from 3′ end; |
| | | 190901947_f | Full PS; 2′MOE |
| 282 | TTTACACAGGACTGAAGACA | ENST00000320717.8_ | 20mer; Gapmer; 5-10-5, PO after 2nd base |
| | | exon_50_chr2_ | from 5′ end, PO after 3rd base from 3′ end; |
| | | 190905004_f | Full PS; 2′MOE |
| 283 | TCAAAGGTTTTACACAGGAC | ENST00000320717.8_ | 20mer; Gapmer; 5-10-5, PO after 2nd base |
| | | exon_50_chr2_ | from 5′ end, PO after 3rd base from 3′ end; |
| | | 190905004_f | Full PS; 2′MOE |
| 284 | CATTAACAGCAATGGCATAT | ENST00000320717.8_ | 20mer; Gapmer; 5-10-5, PO after 2nd base |
| | | exon_50_chr2_ | from 5′ end, PO after 3rd base from 3′ end; |
| | | 190905004_f | Full PS; 2′MOE |
| 285 | CCAGCATTTACCATAGGATT | ENST00000320717.8_ | 20mer; Gapmer; 5-10-5, PO after 2nd base |
| | | exon_60_chr2_ | from 5′ end, PO after 3rd base from 3′ end; |
| | | 190910263_f | Full PS; 2′MOE |
| 286 | TCAGACTGAAACCTGGAAGA | ENST00000320717.8_ | 20mer; Gapmer; 5-10-5, PO after 2nd base |
| | | exon_9_0_chr2_ | from 5′ end, PO after 3rd base from 3′ end; |
| | | 190923917_f | Full PS; 2′MOE |
| 287 | CCACTTTCTCTTTCAGACTG | ENST00000320717.8_ | 20mer; Gapmer; 5-10-5, PO after 2nd base |
| | | exon_9_0_chr2_ | from 5′ end, PO after 3rd base from 3′ end; |
| | | 190923917_f | Full PS; 2′MOE |
| 288 | TGTGCCTTCTGGAAAACACT | ENST00000320717.8_ | 20mer; Gapmer; 5-10-5, PO after 2nd base |
| | | exon_10_0_chr2_ | from 5′ end, PO after 3rd base from 3′ end; |
| | | 190924543_f | Full PS; 2′MOE |
| 289 | ACCTGGAAGTAGAAGTCTAA | ENST00000320717.8_ | 20mer; Gapmer; 5-10-5, PO after 2nd base |
| | | exon_10_0_chr2_ | from 5′ end, PO after 3rd base from 3′ end; |
| | | 190924543_f | Full PS; 2′MOE |
| 290 | TCACTTCAATGGAGCACAGC | ENST00000320717.8_ | 20mer; Gapmer; 5-10-5, PO after 2nd base |
| | | exon_11_0_chr2_ | from 5′ end, PO after 3rd base from 3′ end; |
| | | 190927306_f | Full PS; 2′MOE |
| 291 | CCATCACACTGGCTGATTCA | ENST00000320717.8_ | 20mer; Gapmer; 5-10-5, PO after 2nd base |
| | | exon_11_0_chr2_ | from 5′ end, PO after 3rd base from 3′ end; |
| | | 190927306_f | Full PS; 2′MOE |
| 292 | CCAGTAATTGGGCAGAAACC | ENST00000320717.8_ | 20mer; Gapmer; 5-10-5, PO after 2nd base |
| | | exon_110_chr2_ | from 5′ end, PO after 3rd base from 3′ end; |
| | | 190927306_f | Full PS; 2′MOE |
| 293 | GACCAGCACATCATACCCAT | ENST00000320717.8_ | 20mer; Gapmer; 5-10-5, PO after 2nd base |
| | | exon_12_0_chr2_ | from 5′ end, PO after 3rd base from 3′ end; |
| | | 190930437_f | Full PS; 2′MOE |
| 294 | GAATTCCCTTAACACTGTTG | ENST00000320717.8_ | 20mer; Gapmer; 5-10-5, PO after 2nd base |
| | | exon_12_0_chr2_ | from 5′ end, PO after 3rd base from 3′ end; |
| | | 190930437_f | Full PS; 2′MOE |
| 295 | TTACACAGAGAAACAAGATC | ENST00000320717.8_ | 20mer; Gapmer; 5-10-5, PO after 2nd base |
| | | exon_13_0_chr2_ | from 5′ end, PO after 3rd base from 3′ end; |
| | | 190931545_f | Full PS; 2′MOE |
| 296 | GCAAAGTGTCTCAAATTATC | ENST00000320717.8_ | 20mer; Gapmer; 5-10-5, PO after 2nd base |
| | | exon_13_0_chr2_ | from 5′ end, PO after 3rd base from 3′ end; |
| | | 190931545_f | Full PS; 2′MOE |
| 297 | CCTTTGATCACCACCTTCTC | ENST00000320717.8_ | 20mer; Gapmer; 5-10-5, PO after 2nd base |
| | | exon_130_chr2_ | from 5′ end, PO after 3rd base from 3′ end; |
| | | 190931545_f | Full PS; 2′MOE |
| 298 | TCAACCTGGGATCAGACGTT | Exon-3 | 20mer; Gapmer; 5-10-5, PO after 2nd base |
| | | | from 5′ end, PO after 3rd base from 3′ end; |
| | | | Full PS; 2′MOE |
| 299 | AGAACAGCAAATCTTCCAAG | Exon-2 | 20mer; Gapmer; 5-10-5, PO after 2nd base |
| | | | from 5′ end, PO after 3rd base from 3′ end; |
| | | | Full PS; 2′MOE |
| 300 | CACTCTTTCAACCTGGGA | Exon_3 | 18mer; Gapmer; 4-10-4, PO after 2nd from 5′ |
| | | | end, PO after 3rd position from 3′ end; Full |
| | | | PS; 2′MOE |
| 301 | ATAGAACAGCAAATCTTC | Exon_2 | 18mer; Gapmer; 4-10-4, PO after 2nd from 5′ |
| | | | end, PO after 3rd position from 3' end; Full |
| | | | PS; 2′MOE |
| 302 | GCAATTGTATAGAACAGC | Exon_2 | 18mer; Gapmer; 4-10-4, PO after 2nd from 5′ |
| | | | end, PO after 3rd position from 3′ end; Full |
| | | | PS; 2′MOE |
| 303 | GTATAGAACAGCAAATCT | Exon_2 | 18mer; Gapmer; 4-10-4, PO after 2nd from 5′ |
| | | | end, PO after 3rd position from 3′ end; Full |
| | | | PS; 2′MOE |

In some embodiments, an RNA processing modulator (e.g., an antisense oligonucleotide) comprises at least 18 continuous nucleotides (e.g., comprising or consisting of 18 nucleotides, 19 nucleotides, or 20 nucleotides) of any one of the nucleic acid sequences set forth in SEQ ID NOs: 1-303 (see Column A of Table 1). In some embodiments, an RNA processing modulator consists of 18 continuous nucleotides of any one of the nucleic acid sequences set forth in Column A of Table 1 which are labeled “18mers” in Column B of the same row in Table 1. In some embodiments, an RNA processing modulator comprises 18 continuous nucleotides of any one of the nucleic acid sequences set forth in Column A of Table 1 which are labeled “18mers” in Column B of the same row in Table 1 and comprises one additional nucleotide (either at the 5′ end or 3′ end) or two additional nucleotides (either both at the 5′ end, both at the 3′ end, or one at the 5′ end and the other at the 3′ end) which are complementary to a target sequence in a GLS1 mRNA that hybridizes to the 18 continuous nucleotides of the nucleic acid sequence selected from Column A of Table 1. In some embodiments, an RNA processing modulator consists of 19 continuous nucleotides of any one of the nucleic acid sequences set forth in Column A of Table 1 which are labeled “20mers” in Column B of the same row in Table 1 and comprises one additional nucleotide either at the 5′ end or 3′ end which are complementary to a target sequence in a GLS1 mRNA that hybridizes to the 20 continuous nucleotides of the nucleic acid sequence selected from Column A of Table 1. In some embodiments, an RNA processing modulator comprises or consists of 20 continuous nucleotides of any one of the nucleic acid sequences set forth in Column A of Table 1 which are labeled “20mers” in Column B of the same row in Table 1. In some embodiments, an RNA processing modulator comprises 20 continuous nucleotides of any one of the nucleic acid sequences set forth in Column A of Table 1 which are labeled “20mers” in Column B of the same row in Table 1 and comprises one or more additional nucleotides either at the 5′ end, at the 3′ end, or both the 5′ end and the 3′ end which are complementary to a target sequence in a GLS1 mRNA that hybridizes to the 20 continuous nucleotides of the nucleic acid sequence selected from Column A of Table 1. In some embodiments, an RNA processing modulator comprising at least 18 continuous nucleotides of any one of the nucleic acid sequences set forth in SEQ ID NOs: 1-303 (e.g., an ASO comprising or consisting of 18 nucleotides, 19 nucleotides, or 20 continuous nucleotides of any one of the nucleic acid sequences shown in Column A of Table 1) comprises one or more chemical modifications as set forth in any one of the rows in Column B of Table 1. In some embodiments, an RNA processing modulator comprising at least 18 continuous nucleotides of any one of the nucleic acid sequences set forth in SEQ ID NOs: 1-303 (e.g., an ASO comprising or consisting of 18 nucleotides, 19 nucleotides, or 20 continuous nucleotides of any one of the nucleic acid sequences shown in Column A of Table 1) comprises a pattern of chemical modifications as set forth in any one of the rows in Column B of Table 1. In some embodiments, an RNA processing modulator comprising the at least 18 continuous nucleotides of any one of the nucleic acid sequences of SEQ ID NOs: 1-303 reduces the levels of a GLS1 mRNA (e.g., a mature mRNA or a pre-mRNA) and/or a GLS1 protein by 50% or more (e.g., 50-60%, 60-70%, 70-80%, 80-90%, 90-95%, or 95-100%) in a cell or one or more tissues, such as a cell or one or more tissues (e.g., cerebrospinal fluid, plasma, and/or a brain tissue) in a subject when the RNA processing modulator or a composition thereof is administered to the subject in an effective amount. In some embodiments, the at least 18 continuous nucleotides comprised in an RNA processing modulator are set forth in the nucleic acid sequence of SEQ ID NO: 34 or 233. In some embodiments, an RNA processing modulator comprising the at least 18 continuous nucleotides of the nucleic acid sequence set forth in SEQ ID NO: 34 or 233 reduces the levels of a GLS1 mRNA (e.g., a mature mRNA or a pre-mRNA) and/or a GLS1 protein by 50% or more (e.g., 50-60%, 60-70%, 70-80%, 80-90%, 90-95%, or 95-100%) in a cell or one or more tissues, such as a cell or one or more tissues (e.g., cerebrospinal fluid, plasma, and/or a brain tissue) in a subject when the RNA processing modulator or a composition thereof is administered to the subject in an effective amount.

In some embodiments, an RNA processing modulator comprises or consists of 18 continuous nucleotides, comprises or consists of 19 continuous nucleotides, or comprises or consists of 20 continuous nucleotides of any one of the nucleic acid sequences set forth in SEQ ID NOs: 1-303 (see Column A of Table 1), wherein one or more of positions comprising a “T” residue is substituted for a “U” residue. In some embodiments, an RNA processing modulator comprises or consists of 18 continuous nucleotides, comprises or consists of 19 continuous nucleotides, or comprises or consists of 20 continuous nucleotides of any one of the nucleic acid sequences set forth in SEQ ID NOs: 1-303 (see Column A of Table 1), wherein each position comprising a “T” residue is substituted for a “U” residue. In some embodiments, an RNA processing modulator comprising at least 18 continuous nucleotides of any one of the nucleic acid sequences set forth in SEQ ID NOs: 1-303 (e.g., an ASO comprising or consisting of 18 nucleotides, 19 nucleotides, or 20 continuous nucleotides of any one of the nucleic acid sequences shown in Column A of Table 1), wherein one or more of positions comprising a “T” residue is substituted for a “U” residue and wherein the RNA processing modulator comprises one or more chemical modifications as set forth in any one of the rows in Column B of Table 1. In some embodiments, an RNA processing modulator comprising at least 18 continuous nucleotides of any one of the nucleic acid sequences set forth in SEQ ID NOs: 1-303 (e.g., an ASO comprising or consisting of 18 nucleotides, 19 nucleotides, or 20 continuous nucleotides of any one of the nucleic acid sequences shown in Column A of Table 1), wherein one or more of positions comprising a “T” residue is substituted for a “U” residue and wherein the RNA processing modulator comprises a pattern of chemical modifications as set forth in any one of the rows in Column B of Table 1. In some embodiments, one or more positions in an RNA processing modulator comprising “U” residues comprises an uracil nitrogenous base or a chemically modified uracil nitrogenous base described herein and a deoxyribose sugar or a chemically modified deoxyribose sugar described herein. In some embodiments, the at least 18 continuous nucleotides comprised in an RNA processing modulator are set forth in the nucleic acid sequence of SEQ ID NO: 233, wherein one or more of positions in SEQ ID NO: 233 comprising a “T” residue (e.g., each position in SEQ ID NO: 1 comprising a “T” residue) is substituted for a “U” residue.

In some embodiments, an RNA processing modulator (e.g., an antisense oligonucleotide) comprises a gapmer structure, wherein a region of 10 deoxyribonucleotides is flanked by regions each comprising 4 ribonucleotides (thereby totaling 8 ribonucleotides and 10 deoxyribonucleotides). In some embodiments, 1, 2, 3, or 4 ribonucleotides in each of the regions flanking the region of 10 deoxyribonucleotides comprise a 2′-O-methoxyethyl (—OCH₂CH₂OCH₃ (2′ MOE)) modification. In some embodiments, 1, 2, 3, or 4 ribose sugars comprised in each region flanking the region of 10 deoxyribonucleotides is linked by a phosphorothioate linkage or a phosphodiester linkage. In some embodiments, 1-10 deoxyribose sugars (e.g., 1, 2, 3, 4, 5, 6, 7, 8, 9, or 10 deoxyribose sugars) comprised in the region of 10 deoxyribonucleotides is linked by a phosphorothioate linkage. In some embodiments, one or more ribose sugars (e.g., 1, 2, 3, or 4 ribose sugars) comprised in each of the regions flanking the region of 10 deoxyribonucleotides is linked by a phosphorothioate linkage. In some embodiments, 16 out of the 18 positions are linked by phosphorothioate linkages. In some embodiments, 16 out of the 18 positions are linked by phosphorothioate linkages, wherein the second position is linked to the third position (relative to the 5′ terminal end) by a phosphodiester linkage and the sixteenth position is linked to the seventeenth position (relative to the 5′ terminal end) by a phosphodiester linkage. In some embodiments, an RNA processing modulator (e.g., an antisense oligonucleotide) comprising the gapmer structure comprises or consists of 18 continuous nucleotides of any one of the nucleic acid sequences set forth in SEQ ID NOs: 1-303 (see Column A of Table 1). In some embodiments, an RNA processing modulator (e.g., an antisense oligonucleotide) comprising the gapmer structure comprises or consists of 18 continuous nucleotides of the nucleic acid sequence of SEQ ID NO: 33. In some embodiments, an RNA processing modulator comprising the gapmer structure reduces the levels of a GLS1 mRNA (e.g., a mature mRNA or a pre-mRNA) and/or a GLS1 protein by 50% or more (e.g., 50-60%, 60-70%, 70-80%, 80-90%, 90-95%, or 95-100%) in a cell or one or more tissues, such as a cell or one or more tissues (e.g., cerebrospinal fluid, plasma, and/or a brain tissue) in a subject when the RNA processing modulator or a composition thereof is administered to the subject in an effective amount.

In some embodiments, an RNA processing modulator (e.g., an antisense oligonucleotide) comprises a gapmer structure, wherein a region of 10 deoxyribonucleotides is flanked by regions each comprising 5 ribonucleotides (thereby totaling 10 ribonucleotides and 10 deoxyribonucleotides). In some embodiments, 1, 2, 3, 4, or 5 ribonucleotides in each of the regions flanking the region of 10 deoxyribonucleotides comprise a 2′-O-methoxyethyl (—OCH₂CH₂OCH₃ (2′ MOE)) modification. In some embodiments, 1, 2, 3, 4, or 5 ribose sugars comprised in each region flanking the region of 10 deoxyribonucleotides is linked by a phosphorothioate linkage or a phosphodiester linkage. In some embodiments, 1-10 deoxyribose sugars (e.g., 1, 2, 3, 4, 5, 6, 7, 8, 9, or 10 deoxyribose sugars) comprised in the region of 10 deoxyribonucleotides is linked by a phosphorothioate linkage. In some embodiments, one or more ribose sugars (e.g., 1, 2, 3, 4, or 5 ribose sugars) comprised in each of the regions flanking the region of 10 deoxyribonucleotides is linked by a phosphorothioate linkage. In some embodiments, 18 out of the 20 positions are linked by phosphorothioate linkages. In some embodiments, 18 out of the 20 positions are linked by phosphorothioate linkages, wherein the second position and third position (relative to the 5′ terminal end) are linked by a phosphodiester linkage and the seventeenth position and eighteenth position (relative to the 5′ terminal end) are linked by a phosphodiester linkage. In some embodiments, an RNA processing modulator (e.g., an antisense oligonucleotide) comprising the gapmer structure comprises or consists of 20 continuous nucleotides of any one of the nucleic acid sequences set forth in SEQ ID NOs: 1-303 (see Column A of Table 1). In some embodiments, an RNA processing modulator (e.g., an antisense oligonucleotide) comprising the gapmer structure comprises or consists of 20 continuous nucleotides of the nucleic acid sequence of SEQ ID NO: 233. In some embodiments, an RNA processing modulator comprising the gapmer structure reduces the levels of a GLS1 mRNA (e.g., a mature mRNA or a pre-mRNA) and/or a GLS1 protein by 50% or more (e.g., 50-60%, 60-70%, 70-80%, 80-90%, 90-95%, or 95-100%) in a cell or one or more tissues, such as a cell or one or more tissues (e.g., cerebrospinal fluid, plasma, and/or a brain tissue) in a subject when the RNA processing modulator or a composition thereof is administered to the subject in an effective amount.

In some embodiments, an isolated nucleic acid (e.g., an RNA processing modulator, such as an antisense oligonucleotide) comprises the nucleic acid sequence set forth in SEQ ID NO: 1 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 2 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 3 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 4 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 5 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 6 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 7 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 8 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 9 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 10 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 11 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 12 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 13 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 14 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 15 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 16 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 17 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 18 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 19 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 20 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 21 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 22 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 23 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 24 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 25 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 26 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 27 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 28 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 29 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 30 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 31 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 32 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 33 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 34 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 35 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 36 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 37 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 38 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 39 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 40 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 41 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 42 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 43 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 44 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 45 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 46 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 47 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 48 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 49 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 50 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 51 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 52 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 53 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 54 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 55 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 56 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 57 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 58 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 59 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 60 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 61 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 62 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 63 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 64 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 65 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 66 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 67 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 68 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 69 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 70 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 71 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 72 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 73 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 74 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 75 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 76 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 77 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 78 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 79 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 80 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 81 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 82 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 83 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 84 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 85 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 86 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 87 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 88 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 89 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 90 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 91 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 92 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 93 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 94 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 95 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 96 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 97 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 98 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 99 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 100 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 101 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 102 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 103 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 104 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 105 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 106 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 107 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 108 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 109 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 110 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 111 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 112 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 113 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 114 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 115 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 116 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 117 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 118 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 119 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 120 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 121 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 122 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 123 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 124 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 125 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 126 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 127 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 128 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 129 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 130 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 131 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 132 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 133 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 134 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 135 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 136 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 137 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 138 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 139 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 140 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 141 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 142 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 143 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 144 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 145 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 146 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 147 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 148 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 149 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 150 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 151 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 152 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 153 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 154 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 155 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 156 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 157 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 158 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 159 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 160 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 161 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 162 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 163 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 164 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 165 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 166 and the following modification pattern: Full PS; 2′MOE; 20mer; Gapmer, 5-10-5, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 167 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 168 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 169 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 170 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 171 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 172 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 173 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 174 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 175 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 176 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 177 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 178 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 179 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 180 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 181 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 182 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 183 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 184 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 185 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 186 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 187 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 188 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 189 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 190 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 191 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 192 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 193 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 194 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 195 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 196 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 197 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 198 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 199 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 200 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 201 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 202 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 203 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 204 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 205 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 206 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 207 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 208 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 209 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 210 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 211 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 212 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 213 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 214 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 215 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 216 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 217 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 218 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 219 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 220 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 221 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 222 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 223 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 224 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 225 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 226 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 227 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 228 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 229 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 230 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 231 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 232 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 233 and the following modification pattern: Full PS; 2′MOE; 20mer; 5-10-5; Gapmer, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 234 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 235 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 236 and the following modification pattern: Full PS; 2′MOE; 20mer; Gapmer, 5-10-5, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 237 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 238 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 239 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 240 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 241 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 242 and the following modification pattern: Full PS; 2′MOE; 20mer; Gapmer, 5-10-5, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 243 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 244 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 245 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 246 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 247 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 248 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 249 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 250 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 251 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 252 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 253 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 254 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 255 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 256 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 257 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 258 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 259 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 260 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 261 and the following modification pattern: Full PS; 2′MOE; 20mer; Gapmer, 5-10-5, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 262 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 263 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 264 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 265 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 266 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 267 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 268 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 269 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 270 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 271 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 272 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 273 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 274 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 275 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 276 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 277 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 278 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 279 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 280 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 281 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 282 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 283 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 284 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 285 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 286 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 287 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 288 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 289 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 290 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 291 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 292 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 293 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 294 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 295 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 296 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 297 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 298 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 299 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 300 and the following modification pattern: 18mer; Gapmer; 4-10-4, PO after 2nd from 5′ end, PO after 3rd position from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 301 and the following modification pattern: 18mer; Gapmer; 4-10-4, PO after 2nd from 5′ end, PO after 3rd position from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 302 and the following modification pattern: 18mer; Gapmer; 4-10-4, PO after 2nd from 5′ end, PO after 3rd position from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 303 and the following modification pattern: 18mer; Gapmer; 4-10-4, PO after 2nd from 5′ end, PO after 3rd position from 3′ end; Full PS; 2′MOE.

### Pharmaceutical Compositions

In some embodiments of the disclosure, RNA processing modulators (e.g., antisense oligonucleotides) are formulated into compositions for therapeutic purposes. In some embodiments, the compositions are designed to enhance the therapeutic effect of the RNA processing modulators, for example by increasing biocompatibility, targeting the RNA processing modulator to a site of interest in vivo, reducing clearance of an isolated nucleic acid (e.g., an antisense oligonucleotide) in vivo, increasing the stability of an isolated nucleic acid (e.g., an antisense oligonucleotide) in vivo, increasing uptake of an isolated nucleic acid (e.g., an antisense oligonucleotide) in target cells, or amplifying the intended effect of an isolated nucleic acid (e.g., an antisense oligonucleotide) in vivo.

In some embodiments, the RNA processing modulator (e.g., antisense oligonucleotide) is provided in combination with a pharmaceutically acceptable carrier. A “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

### Methods and Medical Uses

Aspects of the disclosure relate to methods of modulating transcription, translation, function, or activity of genes associated with glutamate signaling in a cell or subject. Also provided is an RNA processing modulator (e.g., antisense oligonucleotide) described by the disclosure for use as a medicament. The RNA processing modulators (e.g., antisense oligonucleotides) may be used in methods of modulating transcription, translation, function, or activity of genes associated with glutamate signaling, e.g. in a cell or subject. In some embodiments, the methods comprise administering a composition comprising one or more RNA processing modulators (e.g., 1, 2, 3, 4, 5, or more RNA processing modulators, for example 1, 2, 3, 4, 5, or more antisense oligonucleotides) to a cell or subject. In some embodiments, administration of the compositions (e.g., RNA processing modulators) results in decreased sensitivity to glutamate in the cell or subject, or alteration of levels of one or more biological products dependent upon glutamate signaling in the subject. The cell may be in vivo, ex vivo, or in vitro.

For example, in some embodiments, administration of an RNA processing modulator (e.g., an antisense oligonucleotide) targeting GLS1 mRNA results in a decrease of glutamate signaling in the cell or subject. In some embodiments, administration of an RNA processing modulator (e.g., an antisense oligonucleotide) targeting GLS1 mRNA results in an increase of glutamate signaling in the cell or subject. The disclosure is based, in part, on the recognition that contacting a cell or subject with an RNA processing modulator that decreases transcription, translation, function or activity of GLS1 protein results in decreased glutamate signaling and/or reduction of neuroinflammation in the subject.

Accordingly, in some aspects, the disclosure provides a method for decreasing glutamate signaling in a cell or subject, the method comprising administering an isolated nucleic acid as described herein to a subject in need thereof. In some embodiments, the isolated nucleic acid comprises an antisense oligonucleotide comprising the sequence set forth in any one of SEQ ID NOs: 1-303 (provided in Column A of Table 1, optionally comprising one or more modifications in column C of Table 1, and optionally wherein the sequence in Column A and the chemistry in column C are provided in the same row of Table 1). In some embodiments, the isolated nucleic acid (e.g., antisense oligonucleotide) is administered as a monotherapy. In some embodiments, the isolated nucleic acid (e.g., antisense oligonucleotide) is administered as a component of a combination therapy with one or more additional therapeutic agents (e.g., one or more selective serotonin reuptake inhibitors (SSRIs), other antidepressants, or antipsychotics).

Generally, it is desirable to decrease glutamate signaling (e.g., by reducing GLS1 levels, transcription, splicing, and/or translation) in certain subjects (e.g., subjects having certain psychiatric diseases or disorders, for example schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.). However, it should be appreciated that, in some embodiments, the disclosure provides a method for increasing glutamate signaling in a cell or subject (e.g., by increasing GLS1 levels, transcription, splicing, and/or translation), the method comprising administering an isolated nucleic acid as described herein to a subject in need thereof. In some embodiments, the isolated nucleic acid comprises an antisense oligonucleotide comprising the sequence set forth in any one of SEQ ID NOs: 1-303.

In some embodiments, an isolated nucleic acid (e.g., antisense oligonucleotide) binds to an mRNA expressed from a particular allele of GLS1 (e.g., binds to a target mRNA in an allele-specific manner).

In some aspects, RNA processing modulators (e.g., antisense oligonucleotides) described by the disclosure are useful for treating a disease or disorder associated with dysregulation of glutamate signaling. Thus, provided herein are RNA processing modulators (e.g., antisense oligonucleotides) described by the disclosure for use in a method of treating a disease or disorder associated with dysregulation of glutamate signaling. A disease or disorder associated with dysregulation of glutamate signaling refers to a disease or disorder in which the subject (e.g., patient) is 1) characterized as having dysfunctional glutamate signaling, and/or 2) has one or more mutations in one or more genes associated with glutamate signaling, and/or 3) has one or more mutations in one or more genes that are involved in a biological pathway that utilizes glutamate (e.g., release of other neurotransmitters, paracrine signaling, etc.). In some embodiments, a disease associated with dysregulation of glutamate signaling is neuroinflammation. Neuroinflammation generally refers to an innate immune system-driven inflammatory response that is centralized in the tissues of the central nervous system (CNS), for example brain and spinal cord tissue.

Methods of measuring glutamate levels in a subject are known in the art. In some embodiments, a glutamate level of a subject is determined by measuring the concentration of glutamate in a biological sample obtained from the subject, for example a blood sample, serum sample, cerebrospinal fluid (CSF) sample, etc.

In some embodiments, a subject has one or more mutations in a GLS1 gene. In some embodiments, a subject having one or more mutations in a GLS1 gene has (or is at risk of developing) a psychiatric disease or disorder. Methods of detecting mutations in a subject's genes are known in the art and include, for example DNA sequencing, RNA sequencing, microarray analysis, etc.

Accordingly, in some aspects, the disclosure provides a method for treating a disease or disorder associated with glutamate signaling, the method comprising administering an isolated nucleic acid as described herein to a subject in need thereof. Also provided is an RNA processing modulator (e.g., antisense oligonucleotide) described by the disclosure for use in a method of treating a disease or disorder associated with glutamate signaling. The method may comprise administering an isolated nucleic acid as described herein to a subject in need thereof. In some embodiments, the isolated nucleic acid comprises an antisense oligonucleotide comprising the sequence set forth in any one of SEQ ID NOs: 1-303 (provided in Column A of Table 1, optionally comprising one or more modifications in column C of Table 1, and optionally wherein the sequence in Column A and the chemistry in column C are provided in the same row of Table 1). In some embodiments, the disease is schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.).

As used herein “treat” or “treating” refers to preventing or delaying disease onset, reducing or preventing the development of symptoms associated with a disease, reducing the severity of a disease, and/or preventing the worsening of symptoms associated with a disease. Accordingly, in some aspects, the disclosure provides a method for treating a subject having or suspected of having a disease caused by dysregulated glutamate signaling. Treatment of a subject involves administration of a composition to the subject (e.g., an RNA processing modulator, such as an antisense oligonucleotide) as described herein.

As used herein, the term “treating” refers to the application or administration of a composition (e.g., an RNA processing modulator, such as an antisense oligonucleotide as described herein) to a subject who has a disease or disorder associated with high levels of glutamate, or with dysregulation of glutamate signaling, with the purpose to cure, heal, alleviate, relieve, alter, remedy, ameliorate, improve, or affect the disorder, the symptom of the disease, or the predisposition toward the disease.

Alleviating a disease associated with glutamate signaling includes delaying the development or progression of the disease, or reducing disease severity. Alleviating the disease does not necessarily require curative results. As used therein, “delaying” the development of a disease (such as a disease associated with glutamate signaling) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with high glutamate levels and/or dysregulation of glutamate signaling.

A subject may be a human, a mouse, a rat, a pig, a dog, a cat, or a non-human primate. In some embodiments, a subject has or is suspected of having a disease or disorder associated with high glutamate levels and/or dysregulation of glutamate signaling. In some embodiments, a subject having a disease or disorder associated with high glutamate levels and/or dysregulation of glutamate signaling comprises at least one GLS1 allele having a mutation. In some embodiments, a GLS1 allele having a mutation (e.g., associated with high glutamate levels and/or dysregulation of glutamate signaling, for example schizophrenia) comprises a frameshift mutation, a splice site mutation, a missense mutation, a truncation mutation or a nonsense mutation. A subject may have two GLS1 alleles having the same mutations (homozygous state) or two GLS1 alleles having different mutations (compound heterozygous state).

The optimal course of administration or delivery of the RNA processing modulators (e.g., antisense oligonucleotides) of the disclosure may vary depending upon the desired result and/or on the subject to be treated. As used herein “administration” refers to contacting cells with an RNA processing modulator and can be performed in vitro or in vivo. Compositions (e.g., pharmaceutical compositions) provided herein can be administered a number of routes including, but not limited to, by oral administration, intravenous administration (e.g., systemic intravenous injection/administration), administration to the brain and/or spinal cord, intracerebral injection, intraventricular injection, intracerebroventricular (ICV) injection, intracisternal injection, intraparenchymal injection, intrathecal injection, and any combination of the foregoing. In some embodiments, administration comprises administration to cerebral spinal fluid, and/or direct administration to an affected site (e.g., a target tissue, for example central nervous system (CNS) tissue, or peripheral nervous system (PNS) tissue).

In general, the most appropriate route of administration will depend upon a variety of factors including the nature of the agent (e.g., its stability in the environment of the gastrointestinal tract), and/or the condition of the subject (e.g., whether the subject is able to tolerate oral administration, injection, etc.). In some embodiments, administration (e.g., injection) of a compound or pharmaceutical composition is performed on a patient in a Trendelenburg position. In some embodiments, compositions are administered to a subject through only one administration route. In some embodiments, multiple administration routes may be exploited (e.g., serially, or simultaneously) for administration of the composition to a subject.

In some embodiments, it may be desirable to deliver the RNA processing modulators (e.g., antisense oligonucleotides) of the disclosure to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. RNA processing modulators (e.g., antisense oligonucleotides) of the disclosure may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, RNA processing modulators (e.g., antisense oligonucleotides) of the disclosure are administered by intravenous injection. In some embodiments, the RNA processing modulators (e.g., antisense oligonucleotides) of the disclosure are administered by intracerebral injection. In some embodiments, the RNA processing modulators (e.g., antisense oligonucleotides) of the disclosure are administered by intracerebroventricular (ICV) injection. In some embodiments, the RNA processing modulators (e.g., antisense oligonucleotides) of the disclosure are administered by intrathecal injection. In some embodiments, the RNA processing modulators (e.g., antisense oligonucleotides) of the disclosure are administered by intrastriatal injection. In some embodiments, the RNA processing modulators (e.g., antisense oligonucleotides) of the disclosure are delivered by intracranial injection. In some embodiments, the RNA processing modulators (e.g., antisense oligonucleotides) of the disclosure are delivered by cisterna magna injection. In some embodiments, the RNA processing modulators (e.g., antisense oligonucleotides) of the disclosure are delivered by cerebral lateral ventricle injection. The skilled artisan will also recognize that the foregoing administration routes may be combined in a single subject (e.g., a subject may be administered RNA processing modulators (e.g., antisense oligonucleotides) of the disclosure using a combination of two or more of the foregoing techniques).

In some embodiments, an effective amount (e.g., an amount sufficient to increase transcription, translation, function, or activity of a target mRNA) is administered to a subject. In some embodiments, an effective amount of an RNA processing modulator (e.g., antisense oligonucleotide) is an amount sufficient to increase transcription, translation, function, or activity of a target mRNA (e.g., of a desired mutant, variant, and/or allele). In some embodiments, an effective amount of an RNA processing modulator (e.g., antisense oligonucleotide) is an amount sufficient to decrease transcription, translation, function, or activity of a target mRNA (e.g., of an undesired mutant, variant, and/or allele). The effective amount will depend primarily on factors such as the species, age, weight, health of the subject, and the tissue to be targeted, and may thus vary among animal and tissue. In some embodiments, an effective amount can be a combination of an effective dosage, frequency, and duration for administration.

In some embodiments, an effective amount (e.g., an amount sufficient to increase transcription, translation, function, or activity of a target mRNA or an amount sufficient to decrease transcription, translation, function, or activity of a target mRNA) is 1 ng-100 mg. In some embodiments, an effective amount (e.g., an amount sufficient to increase transcription, translation, function, or activity of a target mRNA or an amount sufficient to decrease transcription, translation, function, or activity of a target mRNA) is 1-1000 ng. In some embodiments, an effective amount of (e.g., an amount sufficient to increase transcription, translation, function, or activity of a target mRNA or an amount sufficient to decrease transcription, translation, function, or activity of a target mRNA) is 1-10, 10-50, 50-100, 100-200, 200-300, 300-500, 500-750, or 750-1000 ng. In some embodiments, an effective amount (e.g., an amount sufficient to increase transcription, translation, function, or activity of a target mRNA or an amount sufficient to decrease transcription, translation, function, or activity of a target mRNA) is 0.1 μg-100.0 μg. In some embodiments, an effective amount (e.g., an amount sufficient to increase transcription, translation, function, or activity of a target mRNA or an amount sufficient to decrease transcription, translation, function, or activity of a target mRNA) is 0.1-1.0, 1.0-5.0, 5.0-20.0, 20.0-50.0, or 50.0-100.0 μg. In some embodiments, an effective amount (e.g., an amount sufficient to increase transcription, translation, function, or activity of a target mRNA or an amount sufficient to decrease transcription, translation, function, or activity of a target mRNA) is 1 μg-1000 μg. In some embodiments, an effective amount (e.g., an amount sufficient to increase transcription, translation, function, or activity of a target mRNA or an amount sufficient to decrease transcription, translation, function, or activity of a target mRNA) is 100-250, 250-500, 500-750, or 750-1000 μg. In some embodiments, an effective amount (e.g., an amount sufficient to increase transcription, translation, function, or activity of a target mRNA or an amount sufficient to decrease transcription, translation, function, or activity of a target mRNA) is 0.1-1.0, 1.0-5.0, 5.0-20.0, 20.0-50.0, or 50.0-100.0 mg.

In some embodiments, an effective amount (e.g., an amount sufficient to increase transcription, translation, function, or activity of a target mRNA or an amount sufficient to decrease transcription, translation, function, or activity of a target mRNA) is 0.1-1.0, 1.0-20.0, 20.0-50.0, 50.0-200.0, or 200.0-500.0 mg.

During the course of treatment, administration of the composition may be altered or adjusted accordingly. For example, expression of the protein encoded by the nucleic acid targeted by the isolated nucleic acid of the pharmaceutical composition may be monitored to inform methods of use of the composition. Expression information may be obtained, for example, through measuring changes in the levels of the protein or RNA products of the target nucleic acid. Alternatively, sequencing analyses of the target nucleic acid may be employed to determine if expression changes include alterations in the structure or sequence of the protein or RNA product of the target nucleic acid sequence.

The amount of the composition will vary depending on a number of factors such as, but not limited to, clinical features (e.g., disease severity, rate of disease progression, physical characteristics, etc.) of a subject and the mode of administration. Accordingly, the composition may, in certain instances, be administered once or more than one to a single subject. In certain instances, the composition may be administered to the same subject through different modes or routes at different times during the treatment process.

### EXAMPLES

### Example 1: RNA Processing Modulators (RPMs)

This example describes the use of RNA Processing Modulators (RPMs) for modulating translation of one or more mRNA transcripts in a cell or subject. RPMs function by binding to a target-specific mRNA sequence and altering (e.g., upregulating or down-regulating) translation of protein of the mRNA sequence.

In some embodiments, an RPM is an antisense oligonucleotide (ASO). Antisense oligonucleotides (ASOs) typically range from about 10 to 30 nucleotides in length, and may comprise a non-natural sugar-phosphate backbone (e.g., phosphorodiamidate morpholino backbone, phosphorothioate backbone, etc.) and/or one or more modified sugar moieties (e.g., 2′-O-methoxyethyl ribose (2′-O-MOE) modifications, etc.).

In some embodiments, an RPM (e.g., an ASO) targets a structural element of an mRNA transcript, for example an untranslated region (UTR) to modulate the expression of the target (e.g., the target gene encoding the mRNA transcript) by increasing or decreasing transcription and/or translation of the protein encoded by the mRNA transcript (alternatively referred to as modulating expression in the up or the down direction). In another example, an RPM (e.g., an ASO) may target a regulatory region (and thus interfere with protein binding, such as ribosomal protein binding) of a UTR region to modulate the expression of the target in the up or the down direction. Alternatively, an RPM (e.g., an ASO) may target a splice site (e.g., a splice acceptor site or a splice donor site or one or more nucleotide positions thereof in a UTR region) to modulate the expression of the target in the up or the down direction (and thus generating novel protein variants). Additional examples of structural elements that can be targeted by RPMs (e.g., ASOs) include but are not limited to intronic regulatory sites, exonic regulatory sites, exon-intron boundaries, antisense binding sites of a target mRNA transcript, long-non-coding RNA (LncRNA) binding sites of a target gene, and a retained exon of a canonical mRNA.

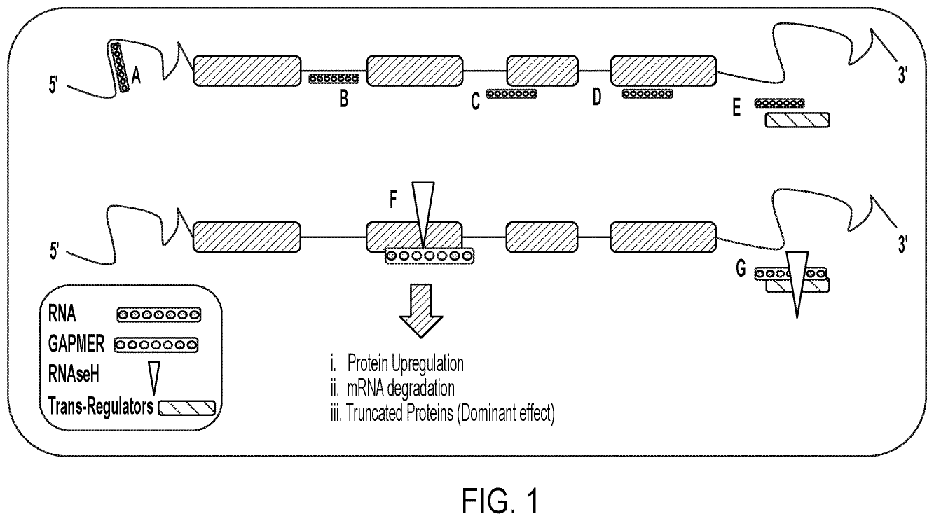

Non-limiting examples of ASOs targeting various structural elements of an mRNA are show in FIG. 1. Composition “A” represents an ASO that binds to the 5′ untranslated region (5′ UTR) of an RNA. Composition “B” represents an ASO that binds to an intron of an RNA. Composition “C” represents an ASO that binds to a splice boundary (e.g., a splice junction) between an exon and intron of an RNA. Composition “D” represents an ASO that binds to an exon (e.g., protein coding region) of an RNA. Composition “E” represents a combination of an ASO binding to a 3′ UTR of an RNA, alone or with a trans-regulator. Composition “F” represents a “gapmer” ASO that binds to an exon (e.g., a protein coding region) of an RNA and mediates RNaseH decay. Composition “G” represents a “gapmer” ASO that binds to a 3′ UTR of an RNA, alone or with a trans-regulator, and mediates RNaseH decay. In some embodiments, ASOs binding to an RNA result in translation of a truncated protein that has a dominant negative effect on the wild-type, full-length protein.

### Example 2: Glutamate Signaling and Psychiatric Disease

This example describes diseases and disorders that area associated with glutamate signaling, particularly psychiatric diseases and disorders and/or diseases associated with neuroinflammation, for example schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.). Glutamate is the most abundant excitatory neurotransmitter in the CNS. One important biosynthetic enzyme for glutamate is glutaminase (GLS1). In presynaptic neurons, glutaminase (GLS1) converts glutamine derived from astrocytes into glutamate, which is subsequently loaded into synaptic vesicles and released from the neuron to elicit an excitatory postsynaptic potential.

Many of the diseases and disorders are associated with aberrant (e.g., abnormal) signaling through glutamate receptors. For example, elevated glutaminase and glutamine synthetase mRNA levels have been observed in the thalamus schizophrenia patients. On the contrary, inhibition of glutaminase activity in the CNS has been observed to produce a “schizophrenia resilient phenotype” in a mouse model. Elevated glutaminase expression has also been associated with neuroinflammation after brain injury. Similarly, treatment with a small molecule glutaminase inhibitor reversed the neuroinflammation. Overexpression of glutaminase has also been reported in microglia of chronic CNS diseases, such as Alzheimer's disease.

### Example 3: ASOs Targeting GLS1

This example describes design of RPMs (e.g., ASOs) that target human GLS1. In the context of diseases associated with certain psychiatric diseases and disorders, it is desirable to decrease protein levels of glutaminase (GLS1) (e.g., by decreasing translation of GLS1 mRNA or by decreasing activity of glutaminase protein). In some embodiments, ASOs are designed to target regions of GLS1 mRNA that will result in increased translation of glutaminase (GLS1) protein and/or increased activity of glutaminase (GLS1) protein.

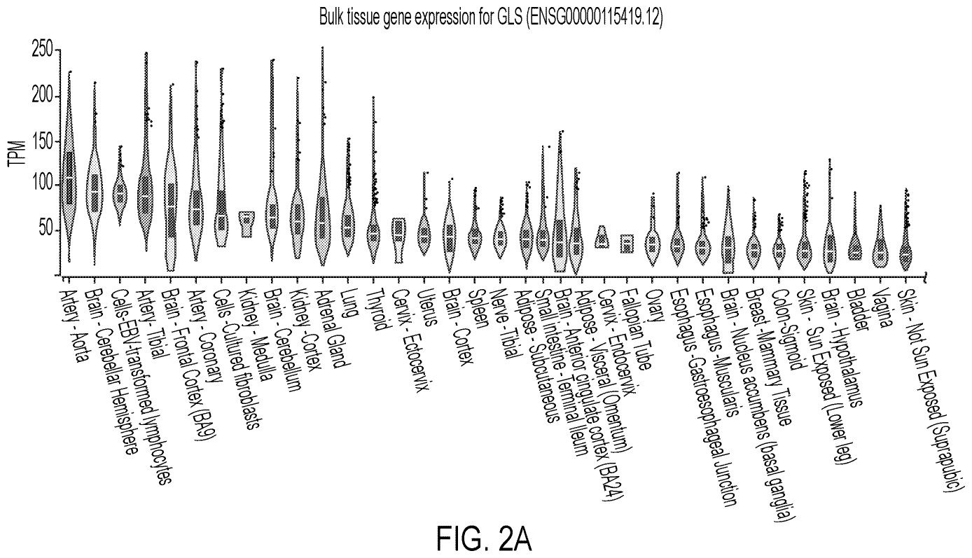

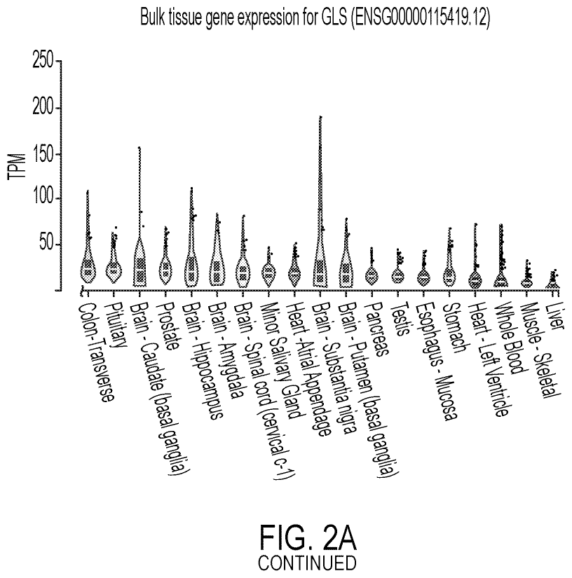

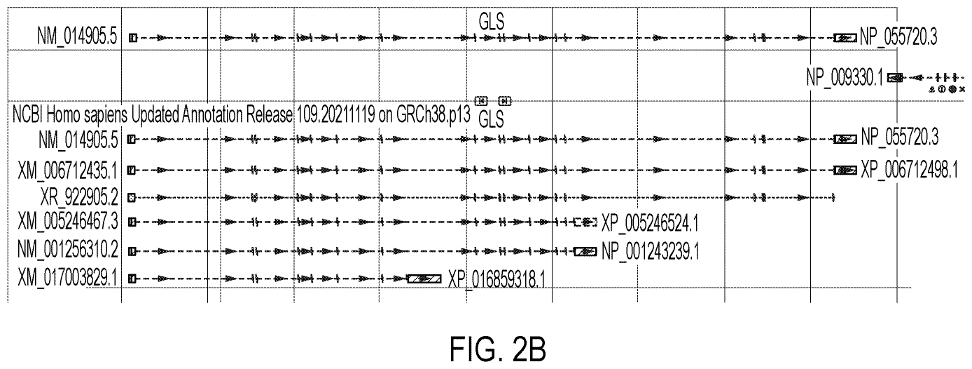

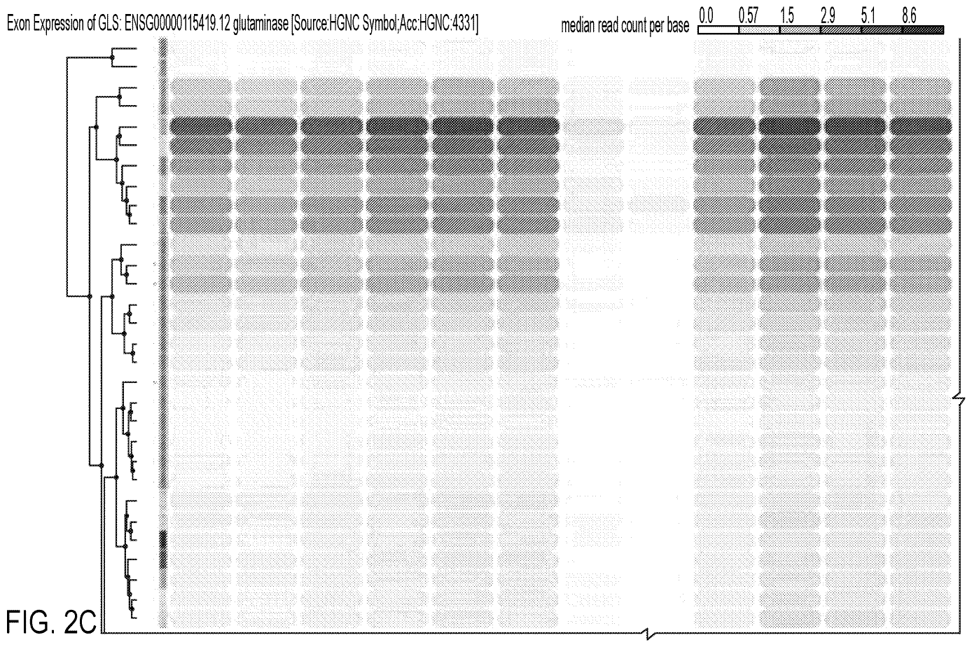

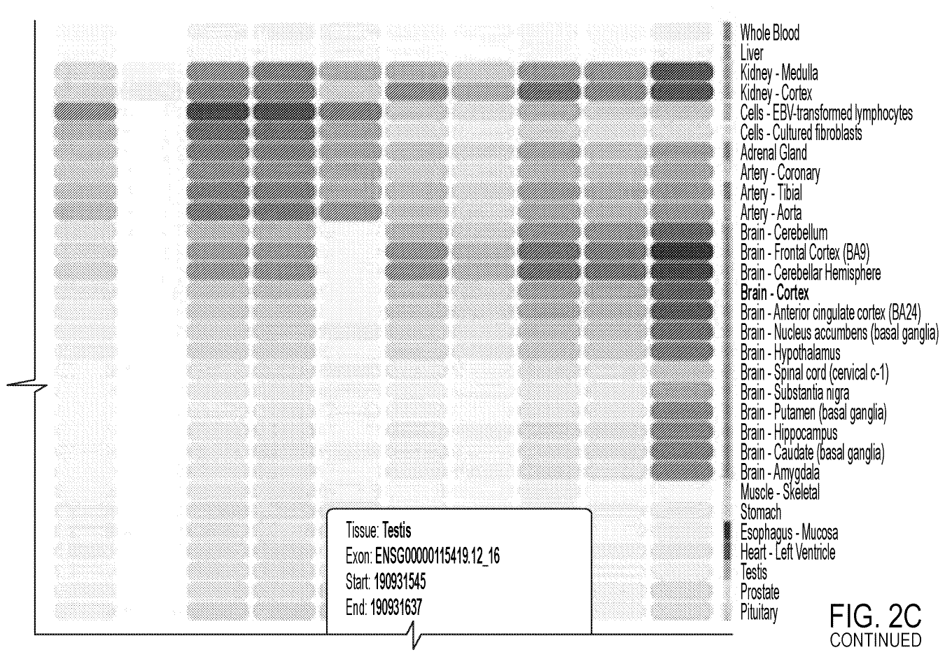

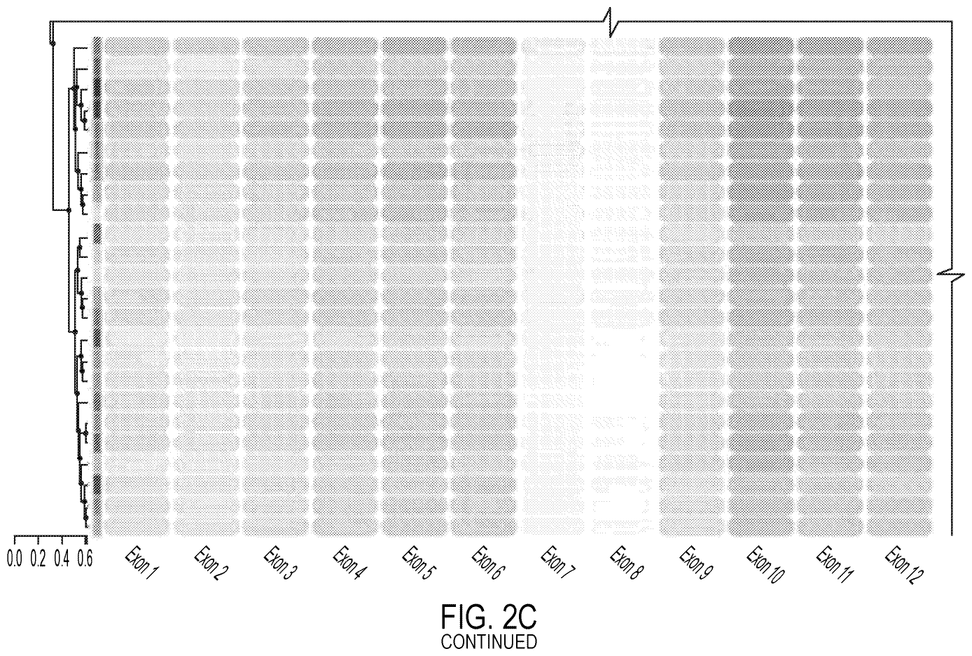

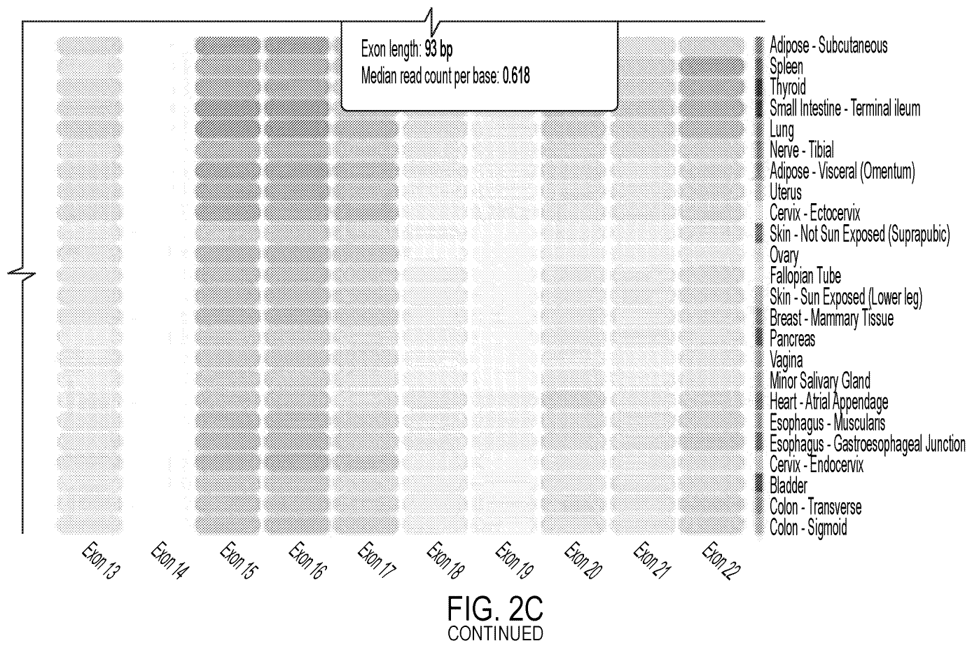

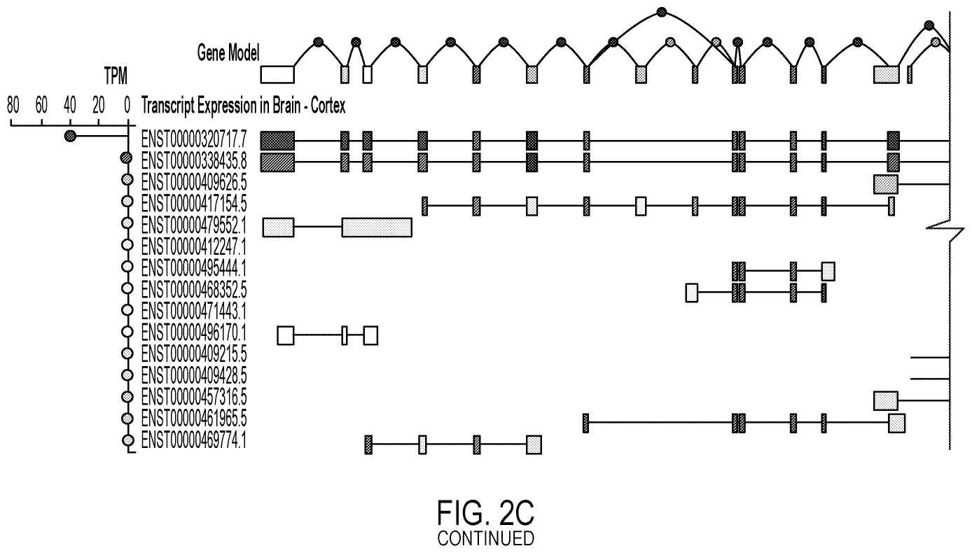

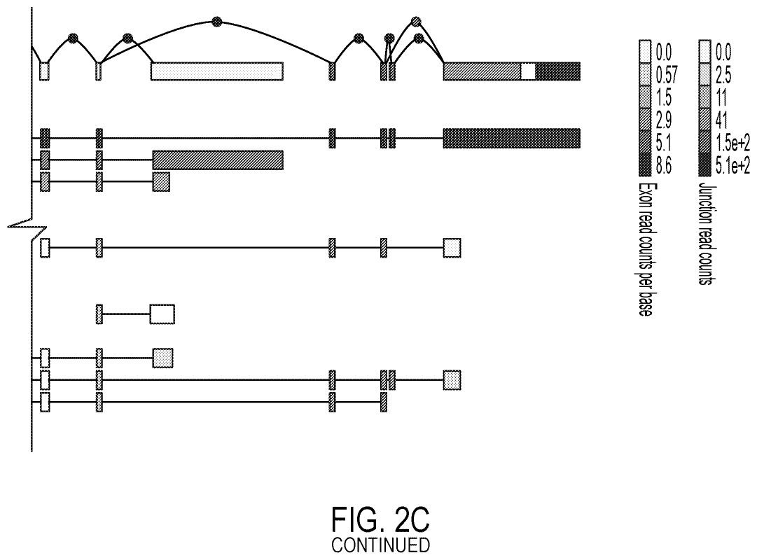

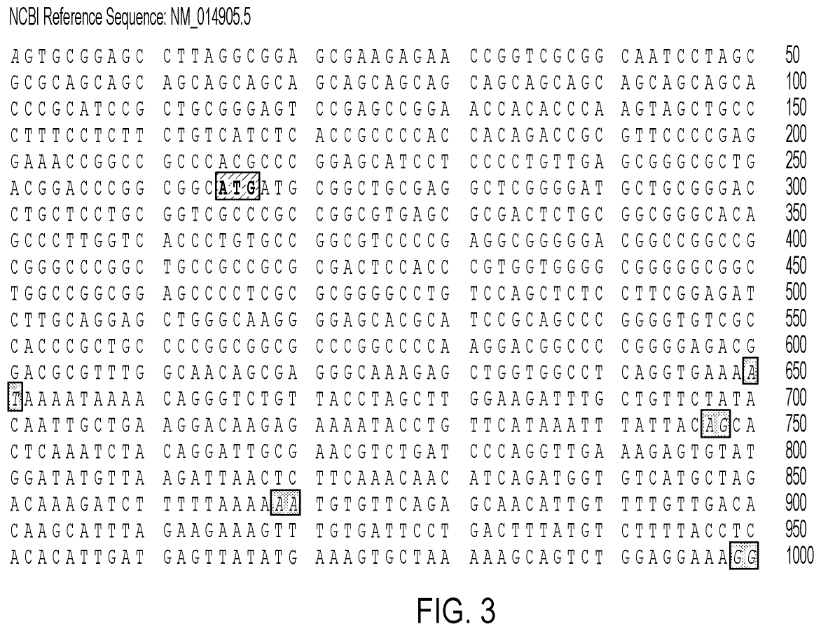

Expression profiling of GLS1 was performed. FIGS. 2A-2C show representative data regarding expression profiling of human glutaminase (GLS1). FIG. 2A shows bulk tissue gene expression of human GLS1; data indicate GLS1 mRNA is expressed in various tissues. FIG. 2B shows a schematic depicting exons and introns present in the GLS1 gene. FIG. 2C shows representative data for exon expression analysis of human GLS1 splice variants in tissue. FIG. 3 is a schematic depicting the primary AUG, and several exon-exon junctions of GLS1 mRNA transcript.

ASOs targeting several of regions of GLS1 were designed, and are described in Table 1. In some embodiments, the ASOs comprise one or more chemical modifications and/or comprise a non-natural sugar-phosphate backbone (e.g., a phosphorothioate backbone). In some embodiments, the ASO has a “gapmer” structure.

### Example 4: In Vitro Screening of ASOs

Cell lines (e.g., U-251 MG human glioblastoma cells) were cultured and maintained using appropriate media (e.g., Dulbecco's Modified Eagle's Medium containing 10% fetal bovine serum). When appropriate, several approaches were used to generate in vitro models for assessment of glutamate signaling. For instance, cell lines may be engineered to stably express glutaminase. When appropriate, glutamate-deficient media was optionally used.

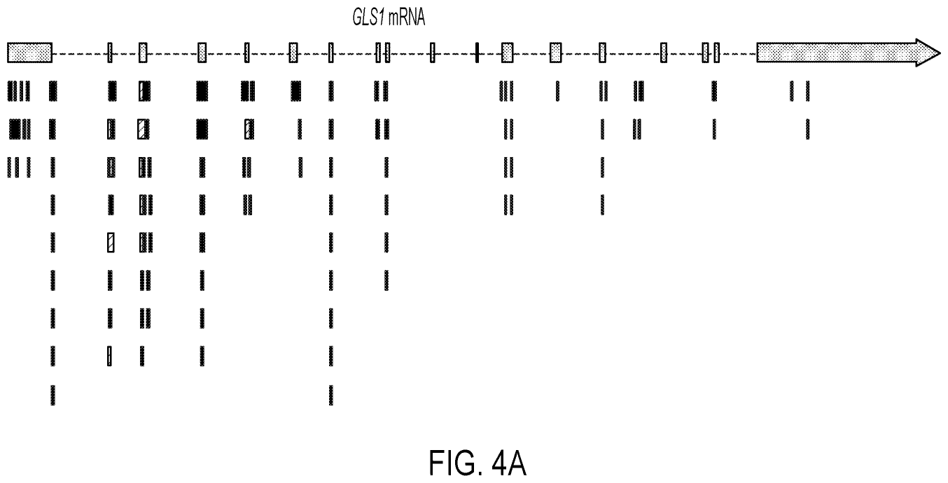

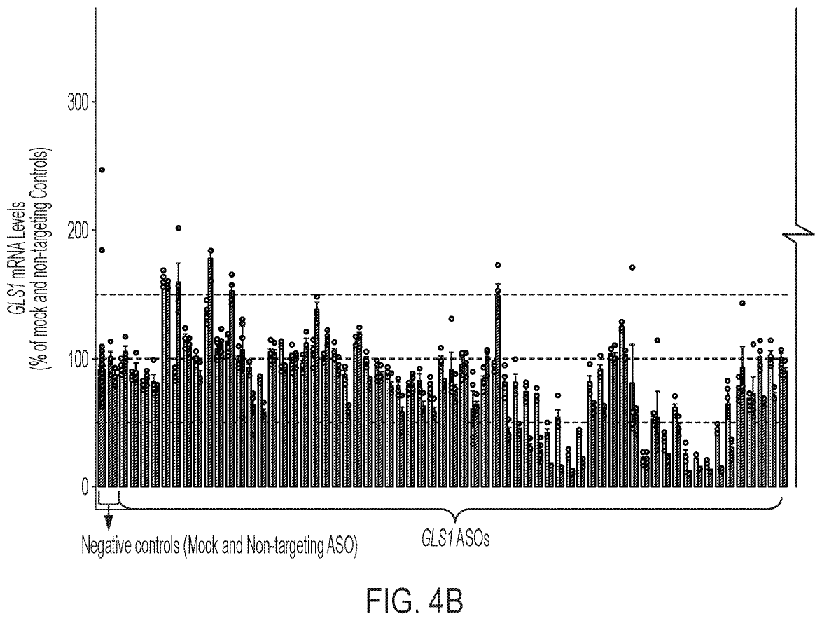

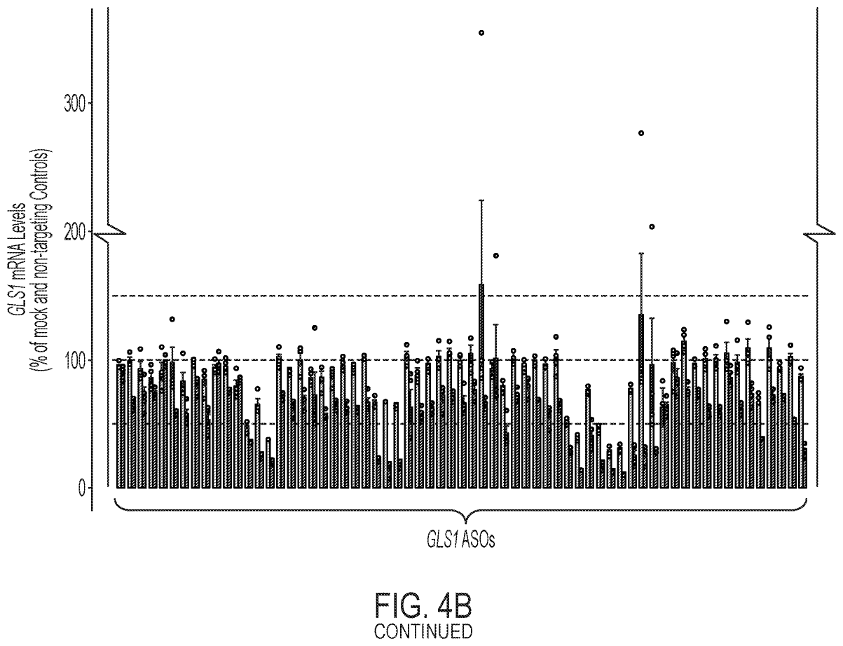

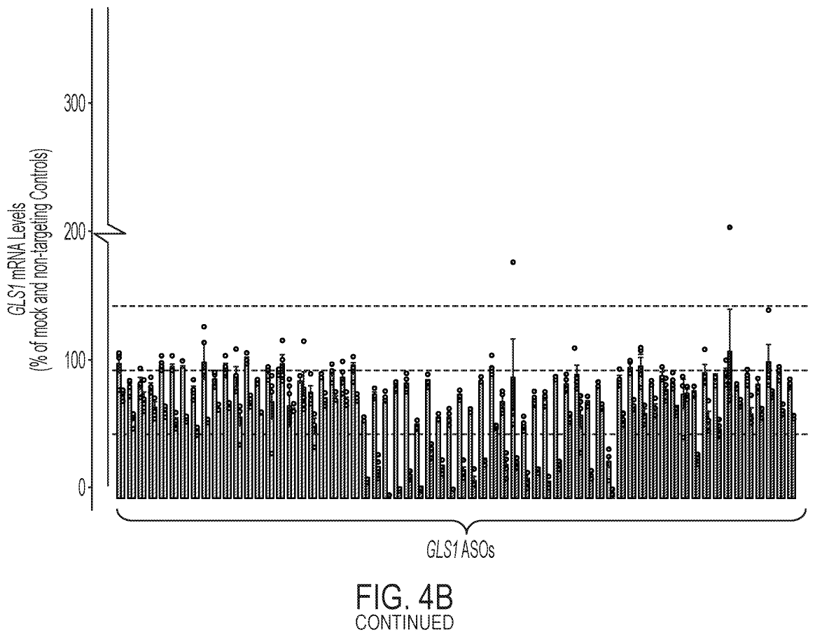

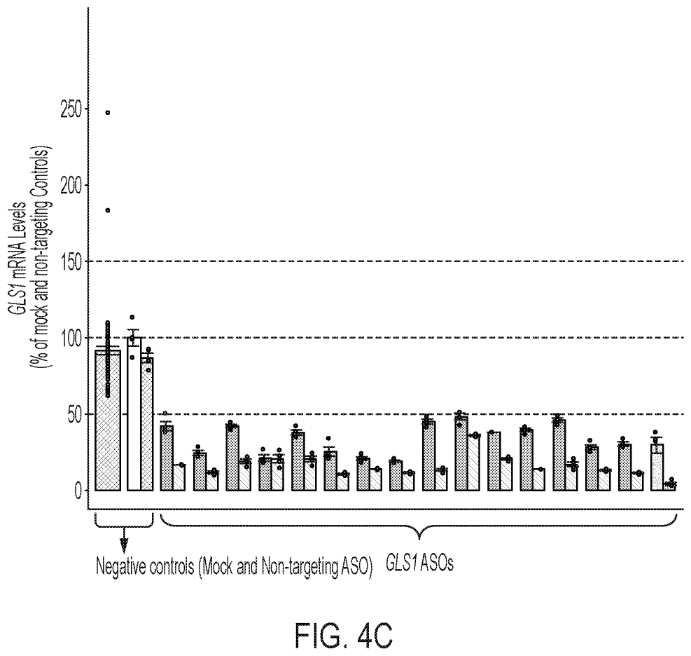

A screen of ASOs targeting GLS1 RNA (Table 1; FIG. 4A) was performed in 96 well plate format, seeding about 20,000 cells per well and treating with the ASOs at different concentrations of 5 nM and 20 nM using the RNAiMAX Lipofectamine protocol. Each concentration was transfected in 4 independent wells for biological quadruplicates. Two different ASO chemistries were assayed for targeting of GLS1 RNA. A non-targeting ASO sequence with matched chemistry and length was used as a negative control, in addition to mock transfected wells treated with PBS or water. Cells were incubated at 37° C. in a cell culture incubator for 48 hours before isolating the total RNA for measurement of gene expression. Total RNA was isolated and evaluated using a branched DNA (bDNA) signal amplification assay in order to quantify GLS1 gene modulation. Cells were lysed and assayed using QuantiGene2.0 bDNA probe sets specific for human GLS1 RNA, as well as for the housekeeping gene glyceraldehyde-3-phosphate dehydrogenase (GAPDH), which was used as an internal control. RNA expression levels were measured and reported as a luminescence signal in arbitrary Relative Light Units (RLUs) for both GLS1 and GAPDH. Measured luminescence levels were then used for analysis.

GLS1 gene expression levels were normalized to both GAPDH and the negative controls. For each sample, GLS1 gene expression levels, provided as RLUs, were first normalized to the housekeeping gene GAPDH (GLS1 RLU/GAPDH RLU). Outliers were detected by testing the value furthest from the mean using the Dixon test on log 2-transformed data within each treatment group per plate. Values with a p-value below 0.01 were removed from subsequent analysis. GLS1 expression relative to controls was then calculated for each sample based on the mean values of the mock and non-targeting control wells within each plate and shown as a percentage ((Sample/Control Mean)*100). Resulting values for all treatment groups are shown in FIG. 4B.

Sixteen (16) ASOs resulted in a decrease in GLS1 RNA expression by more than 50% at the 5 nM dose. The effects of 16 of the most potent ASOs of either ASO chemistry at the 5 nM dose and the 20 nM dose are shown in FIG. 4C.

The effect of these ASOs on glutamate signaling may further be measured by methods known in the art, for example by using radiolabeled glutamate. ASO efficacy may be determined by comparing the levels of glutamate signaling between treated and untreated cells.

To further characterize ASO-dependent changes in glutamate signaling, cytotoxicity may be measured to understand the physiological impact of changes in glutamate levels. Cell viability may be measured by generating survival curves through manual counting of Trypan blue stained cells following ASO treatment. Alternatively, propidium iodide staining of cells followed by flow cytometry analysis may be used to measure cell death.

### Example 5: In Vitro Dose Response of ASOs Targeting GLS1

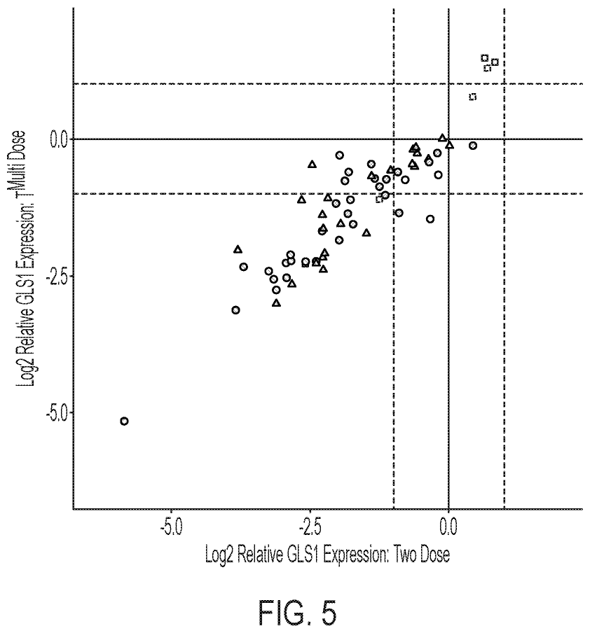

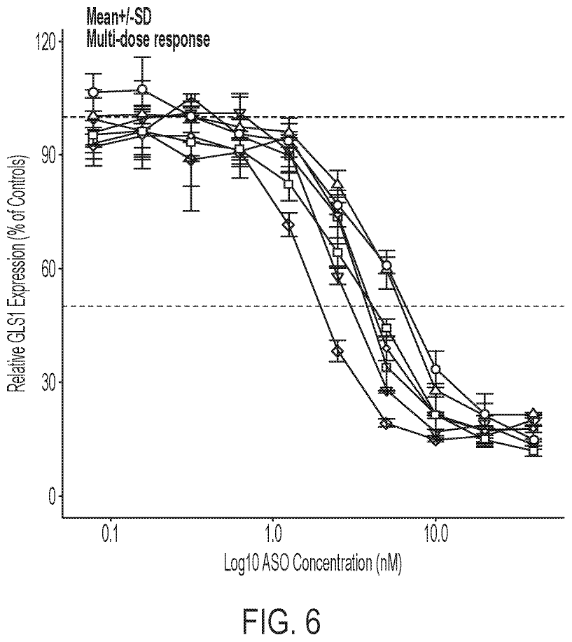

To further characterize ASOs targeting GLS1, select ASOs (e.g., as described in Table 1) were assayed in U251-MG cells over a 10-dose series, and knockdown efficacy was compared to results obtained previously as part of a 2-dose series. To assess the efficacy of GLS1-targeting ASOs in U251-MG cells, 32 ASOs were assayed over a 10-dose series by treating cells with a dosage of 40 nM ASO, 20 nM ASO, 10 nM ASO, 5 nM ASO, 2.5 nM ASO, 1.25 nM ASO, 0.625 nM ASO, 0.3125 nM ASO, 0.156250 nM ASO, or 0.078125 nM ASO. The ASOs included ASOs of either a gapmer chemistry or a skipmer chemistry (alternatively referred to as an “exon-skipper” or “skipper” chemistry herein). U251-MG cells were plated in 96-well plates at 1.5×10⁴ cells per well, then forward transfected with ASOs using the RNAiMAX protocol, with 4 biological replicates per transfection condition. Control cells were reverse transfected with water and a non-targeting control. After incubation at 37° C. for 48 hours, GLS1 gene expression levels were analyzed by a branched DNA (bDNA) assay as described previously. The effect of GLS1-targeting ASOs on GLS1 gene expression was compared against corresponding conditions previously tested in a 2-dose series of U251-MG cells. The tested ASOs were observed to effectively inhibit GLS1 expression at both 20 nM and 5 nM, as in the previous 2-dose series (FIG. 5). Furthermore, the degree of knockdown was determined to be highly correlated between both sets of samples (R=0.88). These results indicate high reproducibility of ASO effectiveness. The efficacy of 7 select skipmer ASOs (e.g., as described in Table 1) (alternatively referred to as ASOs comprising an “exon-skipper” or “skipper” chemistry herein) was specifically examined in the 10-dose series. Each of these skipmer ASOs were observed to effectively inhibit GLS1 expression in U251-MG cells, attaining >50% knockdown between 2.5 nM and 10 nM, with most ASOs attaining >50% knockdown around 5 nM (FIG. 6).

### Example 6: In Vivo ASO Methods

A rodent model of schizophrenia, for example as described by Jones et al. Br J Pharmacol. 2011 October; 164(4): 1162-1194, may be used. Animals are maintained in a consistent light and dark cycle and allowed to acclimate for at least five days prior to experiments. Regular feedings are executed at a consistent time, frequency, and amounts each day. ASOs targeting GLS1 are administered to the animals by infusion. When multiple ASO infusions are performed, administration of the ASO is done at the same time each day to minimize changes in metabolism due to circadian rhythm. ASO infusions are either directly provided to the affected area or into the cerebral spinal fluid (CSF). Animals may be placed in the Trendelenburg position during and after the infusion to aid in distribution of the ASOs into the tissue (e.g., CNS tissue) of the animals. ASOs are solubilized in an appropriate buffer and sterilized prior to infusions. Following infusions, animals are maintained for a predetermined period of time prior to analysis. In some instances, animals are fed a diet with radioactive glutamate to determine the extent of glutamate signaling. To analyze the effect of ASO treatment, animals are anesthetized, and tissue is harvested. Harvested tissue samples are flash frozen in appropriate extraction buffers. Blood samples are isolated, when appropriate, and mixed with buffer for preservation purposes. Harvested tissue samples are cryosectioned and used for immunohistochemistry analysis. Tissue samples are used for measuring glutamate levels.

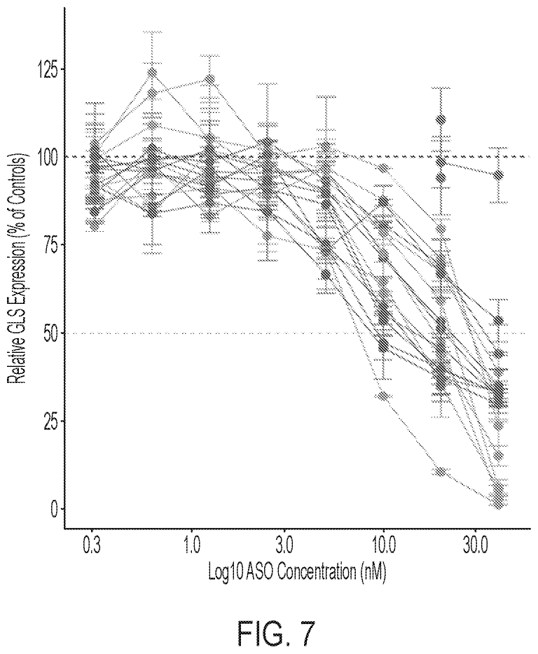

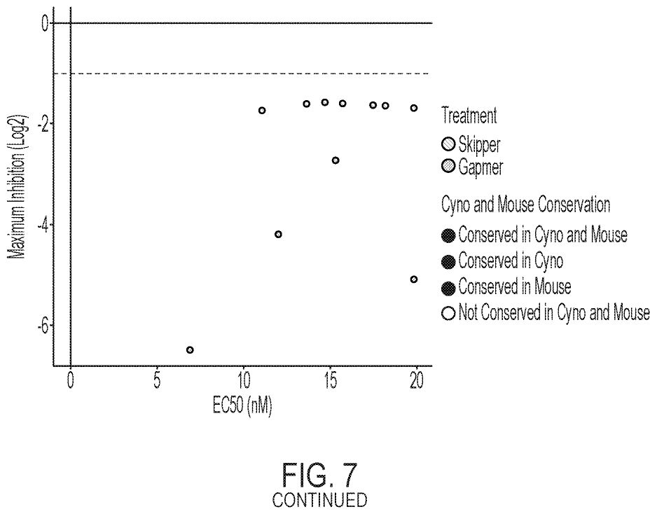

In another experiment, U-251MG cells were transfected using Lipofectamine 3000 with a panel of 15 GLS ASOs with 8 concentrations (40, 20, 10, 5, 2.5, 1.25, 0.625 and 0.3125 nM). 48 h after transfection, RT-qPCR assay was performed to determine normalized expression levels of GLS (Taqman Hs01014020_m1 assay, Thermofisher) using HPRT1 as a normalizer (Taqman Hs02800695_m1 assay, Thermofisher). FIG. 7 (top panel) shows representative data in which GLS mRNA levels are presented relatively to mock-transfected controls in function of the dose of transfected ASOs for the 15 tested GLS ASOs (Skippers (alternatively referred to as ASOs comprising an “exon-skipper” or “skipmer” chemistry herein) and Gapmers are shown). Means are presented, error bars are Standard Error for N=2 biological replicates by group. FIG. 7 (bottom panel) shows representative data for maximum inhibition (log 2, Y-axis) plotted in function of the observed EC50 (X-axis); the most potent ASOs are in the lower left part of the dot-plot.

### Example 7

This example describes in vivo knockdown of GLS1 mRNA using ASOs described herein. Briefly, mice were administered either a single ICV injection, or a repeated ICV injections of the ASOs, and mRNA levels were quantified in the brain tissue. Frozen tissues were lysed and homogenized in RLT buffer using beads (MP Biomedical) before RNA extraction using RNeasy Mini Kit (Qiagen) in a QIAcube station (Qiagen). RNA concentration was evaluated a nanodrop spectrometer (ThermoFisher), integrity using 2100 Bioanalyzer LabChip (Agilent). 500 ng of RNA was reverse-transcribed using SuperScript IV VILO Master Mix ezDNase (Invitrogen). qPCR was performed with TaqMan Fast Advance Master Mix (Invitrogen) in a Quantstudio thermocycler (Applied Biosystems) with 2 independent Taqman (VIC) assays for Gls (IDT Custom GLS-q2, IDT Mm.PT.56a.9691551). Pgk1 and PPIA levels were measured using a Taqman (FAM) assay (Mm00435617_m1, Thermofischer) for normalization using DeltaDeltaCt method.

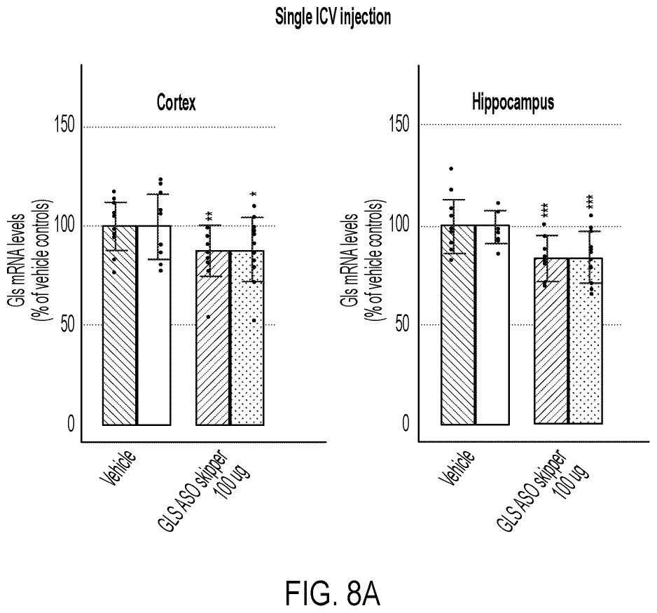

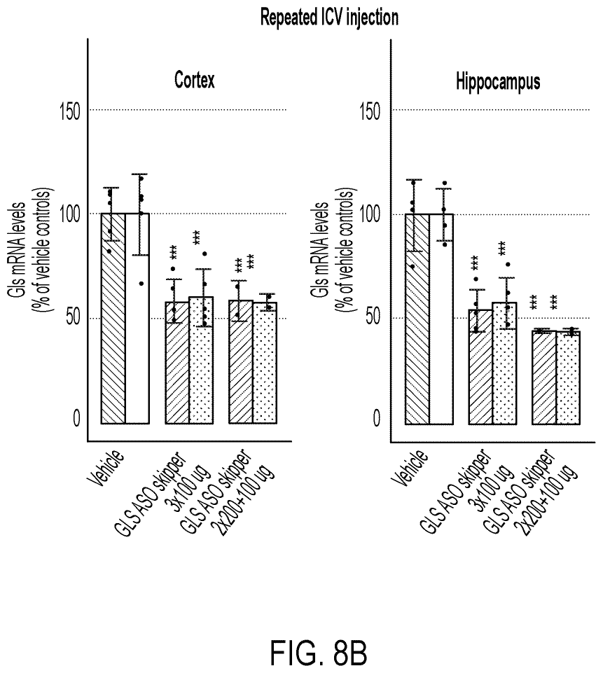

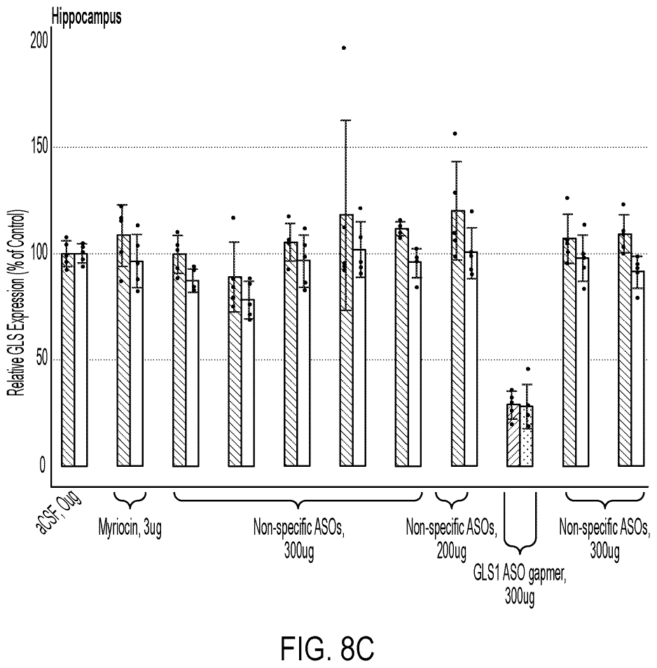

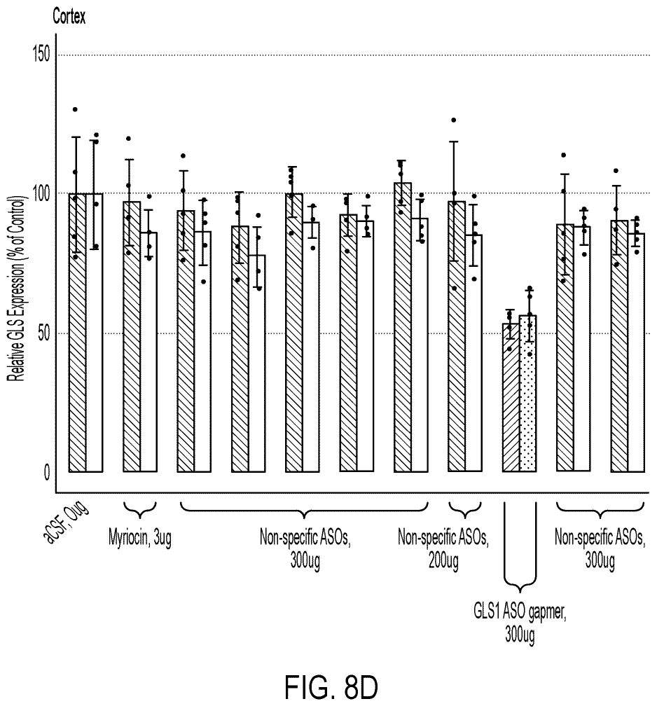

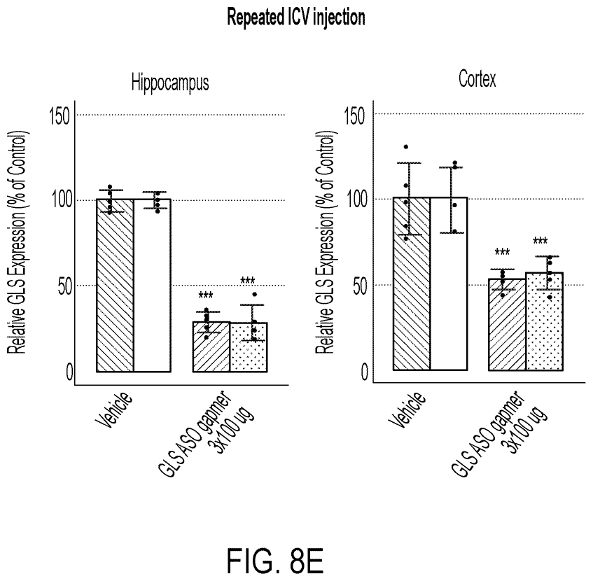

FIGS. 8A-8E show representative data for in vivo reduction of GLS1 mRNA levels in mouse brain. FIG. 8A shows relative GLS1 mRNA levels 3 weeks after a single bilateral stereotaxic ICV injection of vehicle (artificial CSF) or 100 ug of a GLS ASO comprising the nucleotide sequence of SEQ ID NO: 34 as set forth in Column A of row 35 in Table 1 and an exon-skipper chemistry (alternatively referred to as a “skipper” or “skipmer” chemistry herein) according to the chemical modifications set forth in Column C of row 35 in Table 1. FIG. 8B shows relative GLS1 mRNA levels one week after the last of three ICV injections through a canula, with 1 week interval, of vehicle (artificial CSF), 100 ug+100 ug+100 ug or 200 ug+200 ug+100 ug of a GLS1 ASO comprising the nucleotide sequence of SEQ ID NO: 34 as set forth in Column A of row 35 in Table 1 and an exon-skipper chemistry (alternatively referred to as a “skipper” or “skipmer” chemistry herein) according to the chemical modifications set forth in Column C of row 35 in Table 1. FIG. 8C shows relative GLS1 mRNA levels in hippocampus tissues of mouse subjects 7 days after a series of three weekly ICV injections of vehicle (artificial CSF), 3 ug of myriocin, a non-specific ASO at a dose of 200 ug (100 ug+50 ug+50 ug) or 300 ug (100 ug+100 ug+100 ug), or GLS1 ASO comprising the nucleotide sequence of SEQ ID NO: 233, a gapmer structure, and the chemical modifications as set forth in Columns A and C of row 234 of Table 1 at a dose of 300 ug (100 ug+100 ug+100 ug). FIG. 8D shows relative GLS1 mRNA levels in cortex tissues of mouse subjects seven days after a series of three weekly ICV injections of vehicle (artificial CSF), 3 ug of myriocin, a non-specific ASO at a dose of 200 ug (100 ug+50 ug+50 ug) or 300 ug (100 ug+100 ug+100 ug), or GLS1 ASO comprising the nucleotide sequence of SEQ ID NO: 233, a gapmer structure, and the chemical modifications as set forth in Columns A and C of row 234 of Table 1 at a dose of 300 ug (100 ug+100 ug+100 ug). FIG. 8E shows relative GLS1 mRNA levels one week after the last of three ICV injections through a canula, with 1 week interval, of vehicle (artificial CSF) or 100 ug+100 ug+100 ug of a GLS1 ASO comprising the nucleotide sequence of SEQ ID NO: 233, a gapmer structure, and the chemical modifications as set forth in Columns A and C of row 234 of Table 1.

In the repeated ICV injection study, some animals exhibited minor acute in-life observations post GLS1 ASO administration, which were largely resolved by 24 hours. Upon administration of a GLS1 ASO comprising the nucleotide sequence of SEQ ID NO: 34 as set forth in Column A of row 35 in Table 1 and skipper chemistry (alternatively referred to as an “exon-skipper” or “skipmer” chemistry herein) according to the chemical modifications set forth in Column C of row 35 in Table 1, one out of five animals in the 300 ug dose group was sacrificed early, and three out of five animals in the 300 ug dose group died before study completion. No major effects on life were observed in animals that received administration of a GLS1 ASO comprising the nucleotide sequence of SEQ ID NO: 233, a gapmer structure, and the chemical modifications as set forth in Columns A and C of row 234 of Table 1.

Repeated ICV injection of GLS1 ASO comprising the nucleotide sequence of SEQ ID NO: 34 as set forth in Column A of row 35 in Table 1 and an skipper chemistry (alternatively referred to as an “exon-skipper” or “skipmer” chemistry herein) according to the chemical modifications set forth in Column C of row 35 in Table 1 resulted in GLS1 mRNA knockdown at levels of 40% and 53% in the cortex and hippocampus, respectively (FIG. 8B). Repeated ICV injection of GLS1 ASO comprising the nucleotide sequence of SEQ ID NO: 233, a gapmer structure, and the chemical modifications as set forth in Columns A and C of row 234 of Table 1 resulted in GLS1 mRNA knockdown at levels of 40-46% and 74-77% in the cortex and hippocampus, respectively (FIGS. 8C-8E). No significant effects on GLS1 mRNA were observed in subjects that were administered control treatments of vehicle, myriocin, or non-specific ASOs.

### Example 8

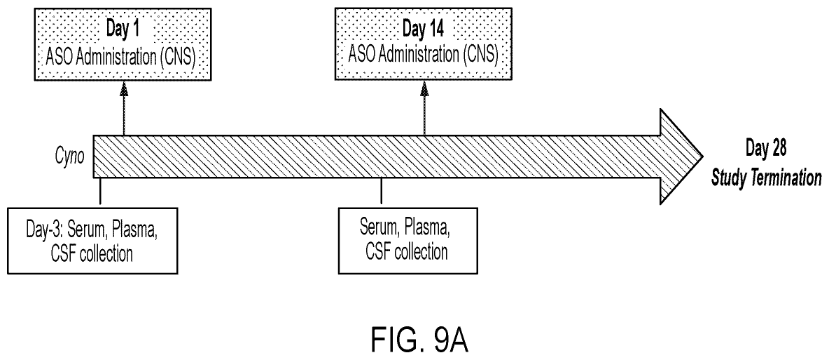

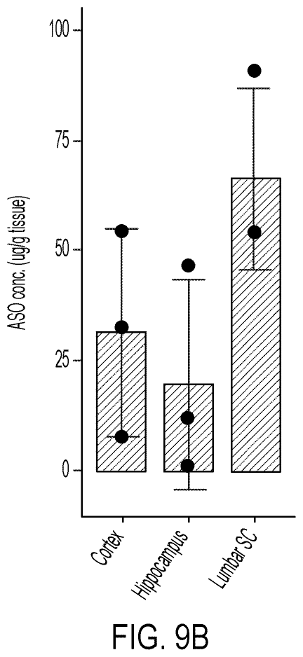

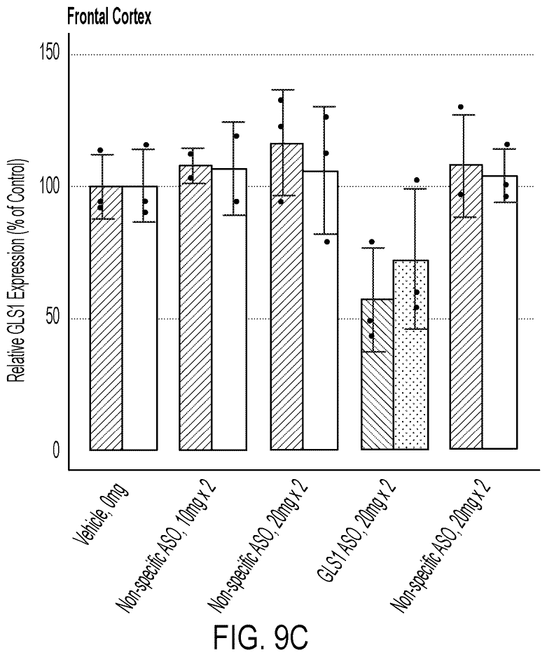

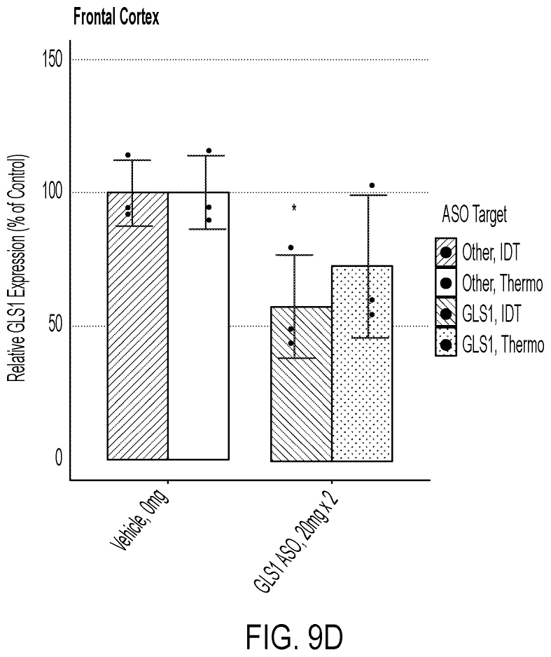

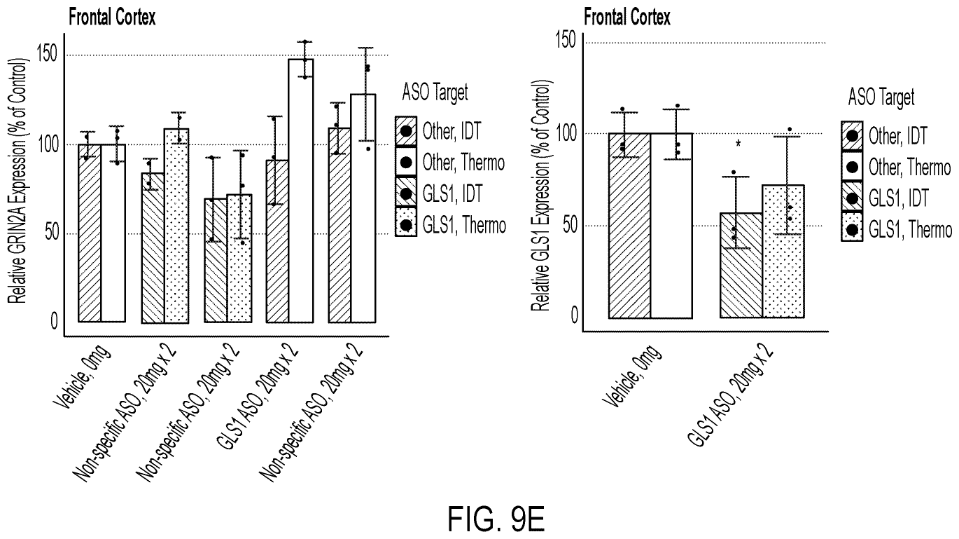

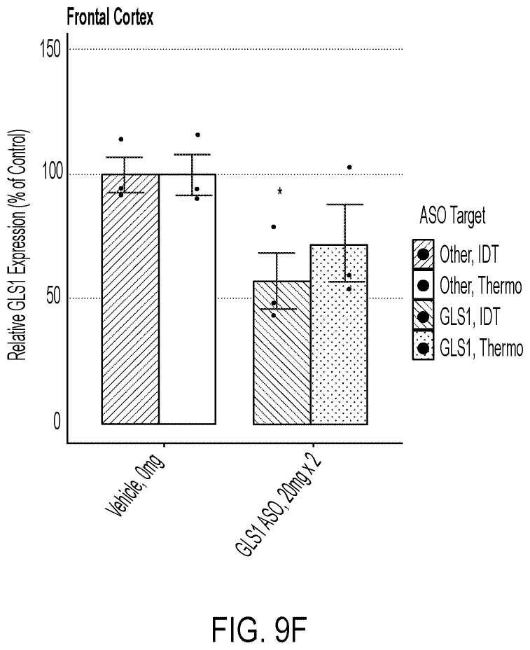

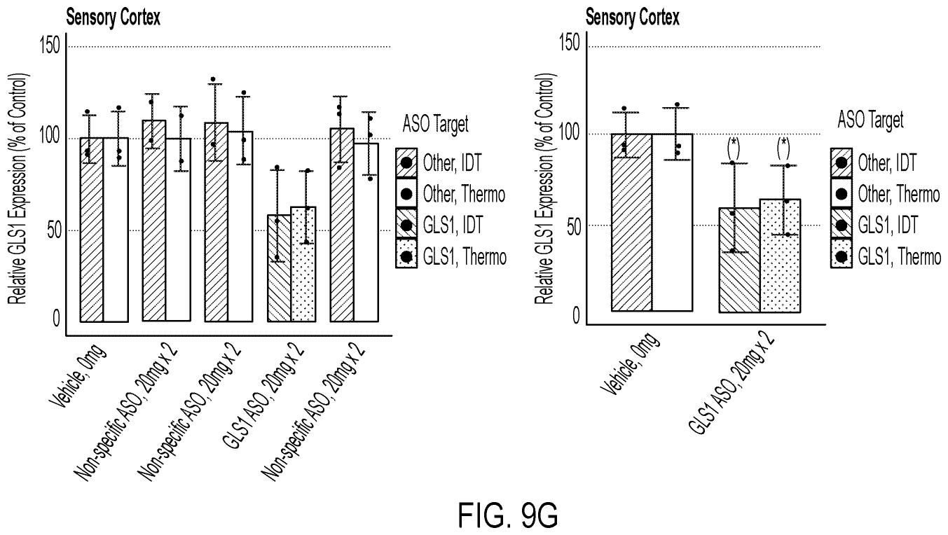

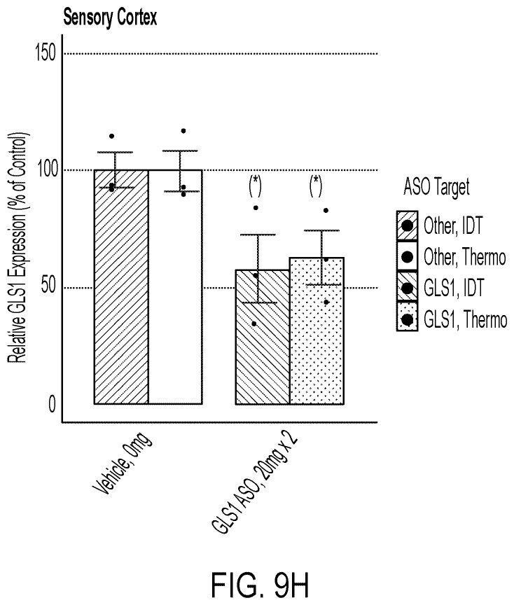

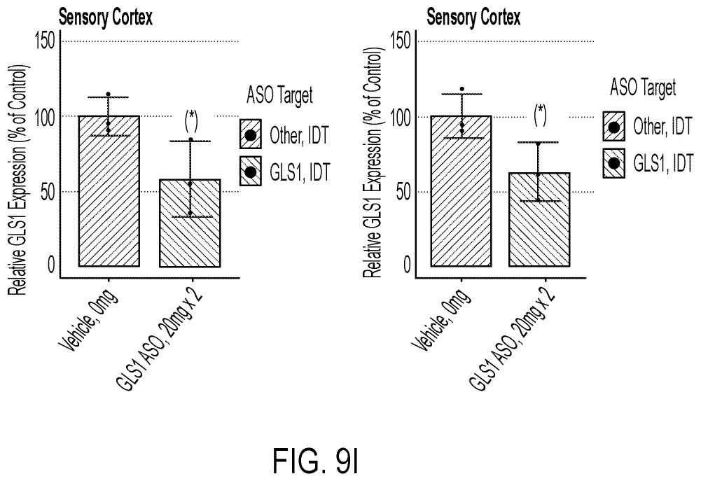

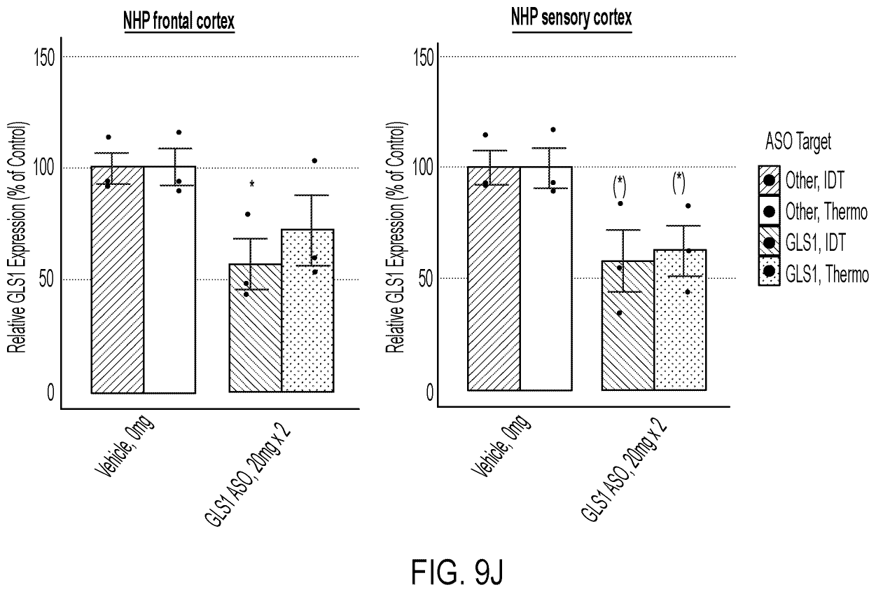

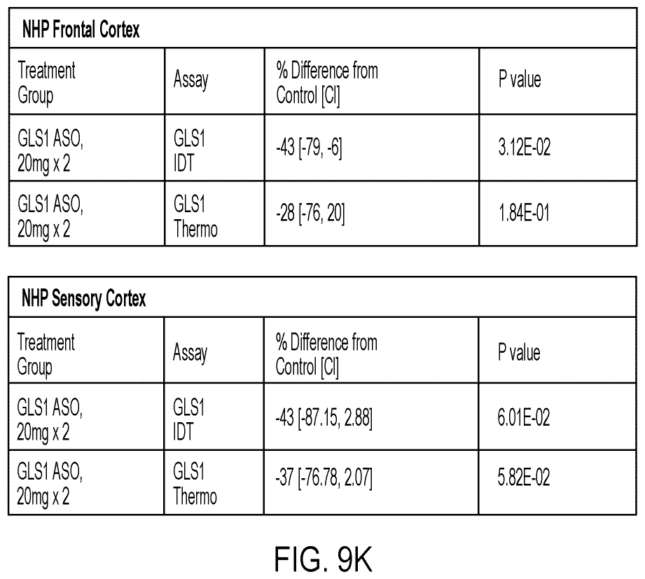

This example describes in vivo administration of GLS1 ASOs to cynomolgus monkey (Macaca fascicularis) subjects (also referred to as “non-human primate subjects). Briefly, non-human primate subjects received two intrathecal (IT) injections of either vehicle (artificial CSF) or GLS1 ASO comprising the nucleotide sequence of SEQ ID NO: 34 as set forth in Column A of row 35 in Table 1 and an exon-skipper chemistry (alternatively referred to as a “skipper” or “skipmer” chemistry herein) according to the chemical modifications set forth in Column C of row 35 in Table 1. Each group (vehicle-injected and ASO-injected) consisted of two male subjects and one female subject, and each round of IT injection was performed two weeks apart (days 1 and 14). In both rounds of ASO IT injection, GLS1 ASO was administered at a dose of 20 mg to yield a total dose of 40 mg of GLS1 ASO administered over a 28-day period. At day 28 (two weeks after the last intrathecal injection) non-human primate subjects were sacrificed and samples of cerebrospinal fluid as well as brain (frontal cortex, sensory cortex, and hippocampus), lumbar spinal cord, dorsal root ganglion, kidney, liver, spleen, heart, stomach, and gonads tissues were collected. Additionally, serum and plasma were collected 3 days prior to day 1 and before day 14 (FIG. 9A). Liquid chromatography-mass spectrometry analysis was used to measure GLS1 ASO pharmacokinetics (ASO levels) and RT-qPCR was used to measure GLS1 ASO pharmacodynamics (GLS1 mRNA expression). Then, GLS1 ASO concentrations in frontal cortex, hippocampus, and lumbar spinal cord tissue samples were quantified to determine GLS1 ASO levels as a result of administration through IT injection.

TABLE 2
GLS1 ASO Levels in Tissue Samples of Non-Human Primate Subjects

| | | | | | | ASO Concentration and | | |
|---|---|---|---|---|---|---|---|---|
| | | | Dose | Dose | | Mean (μg/g tissue) | | |
| | | | 1 | 2 | | | | Lumbar |
| | | Group | (Day | (Day | Total | Frontal | | Spinal |
| Treatment | Chemistry | Size | 1) | 14) | Dose | Cortex | Hippocampus | Cord |
| Vehicle | N/A | 2 male | N/A | N/A | N/A | N/A | N/A | N/A |
| (artificial | | 1 female | | | | | | |
| CSF) | | | | | | | | |
| GLS1 | Skipper** | 1 male | 20 mg | 20 mg | 40 mg | 55, 1, 33 | 47, 1, 12 | 91, 54, 55 |
| ASO | | 2 female | | | | (32) | (20) | (67) |
| (SEQ ID | | | | | | | | |
| NO: 34*) | | | | | | | | |

GLS1 ASO-treated subjects received an ASO comprising the nucleotide sequence of SEQ ID NO: 34* and the chemical modifications** as set forth in Columns A and C, respectively, of row 35 of Table 1

No GLS1 ASO was detected in tissue samples obtained from non-human primate subjects that underwent two IT injections with vehicle. No in-life safety signals were observed in non-human primate subjects who received two IT injections with vehicle or GLS1 ASO.

In tissue samples obtained from non-human primate subjects that underwent two IT injections with ASO targeting GLS1 mRNA, GLS1 ASO was detected at a mean concentration of approximately 20-67 pg/g tissue (FIG. 9B and Table 2). Additionally, ASO pharmacokinetics in tissue samples obtained from non-human primate subjects administered ASO via IT injection met or exceeded previously documented pharmacokinetic data in non-human primate ASO studies (see, e.g., Malat1 ASO administration in Jafar-Nejad et al. (2021). Nucleic Acids Research. 49 (2): 657-673). Further analyses indicated that two IT injections with GLS1 ASO resulted in significant GLS1 mRNA knockdown (~30-40%) in frontal cortex and sensory cortex tissue samples obtained from non-human primate subjects (FIG. 9C-9K). Injection with vehicle (artificial CSF) or a non-specific ASO had no significant effect on GLS1 mRNA knockdown (FIGS. 9D-9K).

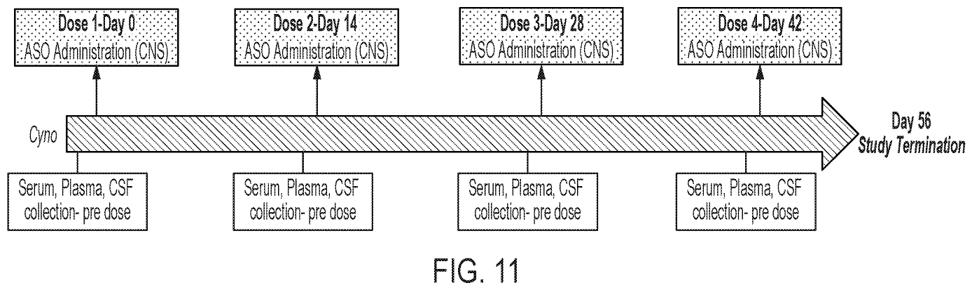

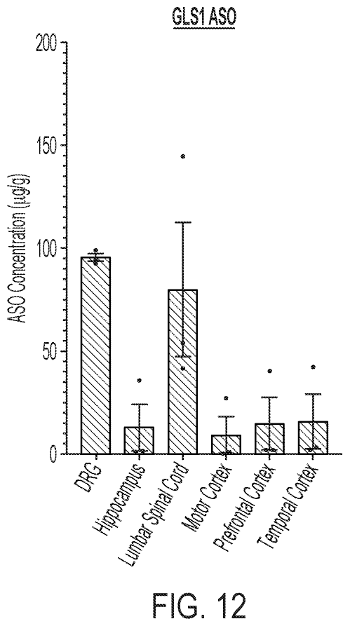

The pharmacokinetic effects associated with a series of four IT injections of ASO were assessed in additional non-human primate subjects. Three male non-human primate subjects were administered a series of four IT injections of either vehicle (artificial CSF) or ASO comprising the nucleotide sequence of SEQ ID NO: 233, a gapmer structure, and the chemical modifications as set forth in Columns A and C of row 234 of Table 1 at a dose of 80 mg (20 mg+20 mg+20 mg+20 mg). For all non-human primate subjects, each round of IT injection was performed two weeks apart (days 0, 14, 28, and 42). Samples of cerebrospinal fluid as well as brain (frontal cortex, sensory cortex, and hippocampus), lumbar spinal cord, dorsal root ganglion, kidney, liver, spleen, heart, stomach, and gonads tissues were collected at two weeks following the last IT injection (FIG. 11). Liquid chromatography-tandem mass spectrometry (LC-MS/MS) analysis was used to measure ASO pharmacokinetics (ASO levels) in tissue samples obtained from injected non-human primates. ASO concentrations in tissue samples were quantified to determine ASO levels as a result of IT injection (FIG. 12).

### Example 9

This Example describes analyses of immunostimulatory effects of GLS1 ASOs in human peripheral blood mononuclear cells (huPBMCs).

All ASOs were prepared using in vivo quality grade material in manner that was consistent with analyses performed in animal subjects as described above. huPBMCs were harvested from healthy donors and went either untreated, treated with a cytokine/chemokine response control agent, or treated with a GLS1 ASO comprising the nucleotide sequence of SEQ ID NO: 233, a gapmer structure, and the chemical modifications as set forth in Columns A and C of row 234 of Table 1 at a concentration of 1 μM, 3 μM, or 10 μM for 24 hours.

Cytokine/chemokine response control agents included: XD-01024, a cholesterol-conjugated ApoB siRNA which has TLR7/8 agonist effects; CL097, a water-soluble derivative of imidazoquinoline compound R848 which is a TLR7/8 ligand; Imiquimod (R837), an immune response modifier having potent antiviral activity and also induces production of cytokines and activates TLR7; ODN2216 a 20mer oligo containing unmethylated CpG and has TLR9 agonist effects; ODN2006 having preference towards TLR9, one or more CpGs, class B; ODN2395, TLR9, CpG with palindromic motif, class C; TL8-506, a benzoazepine compound which has TLR8 agonist effects; LMW poly(l:c) which has TLR3 agonist effects; and XD-00366, a 25mer double-stranded, unmodified, blunt-ended LacZ RNA duplex which has TLR7/8 agonist effects.

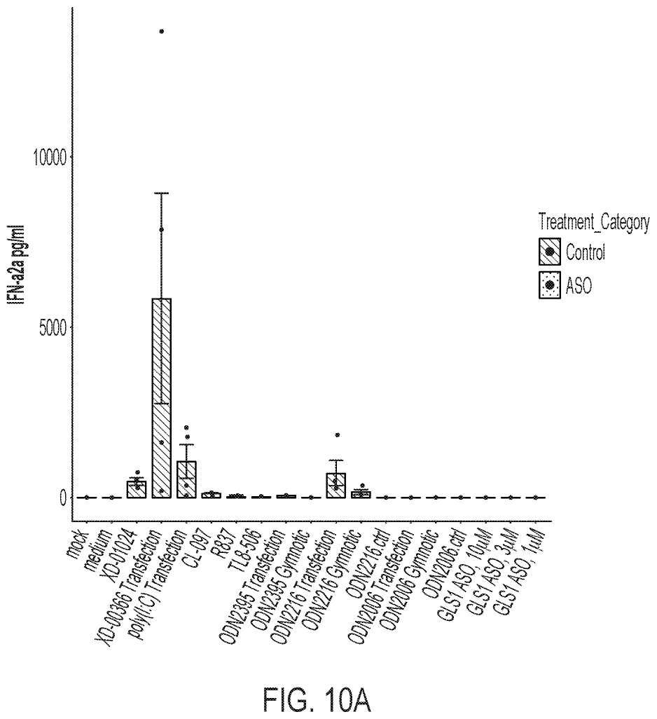

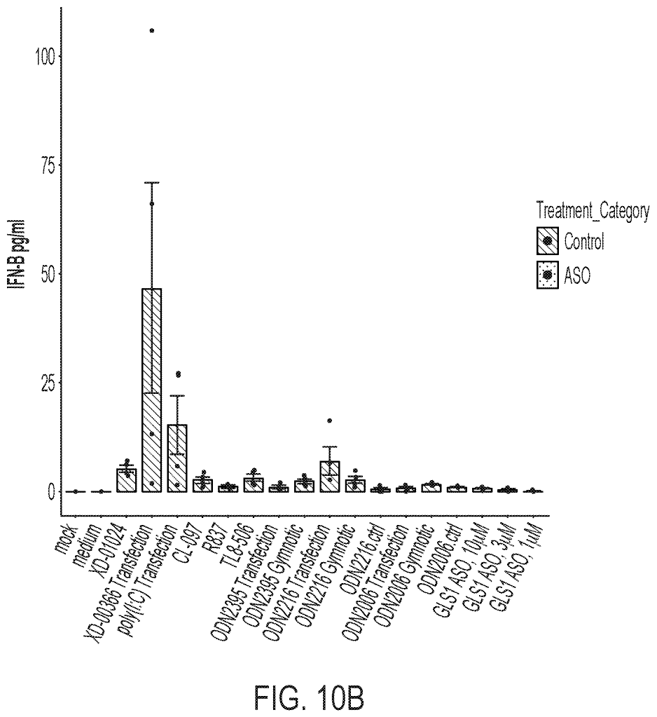

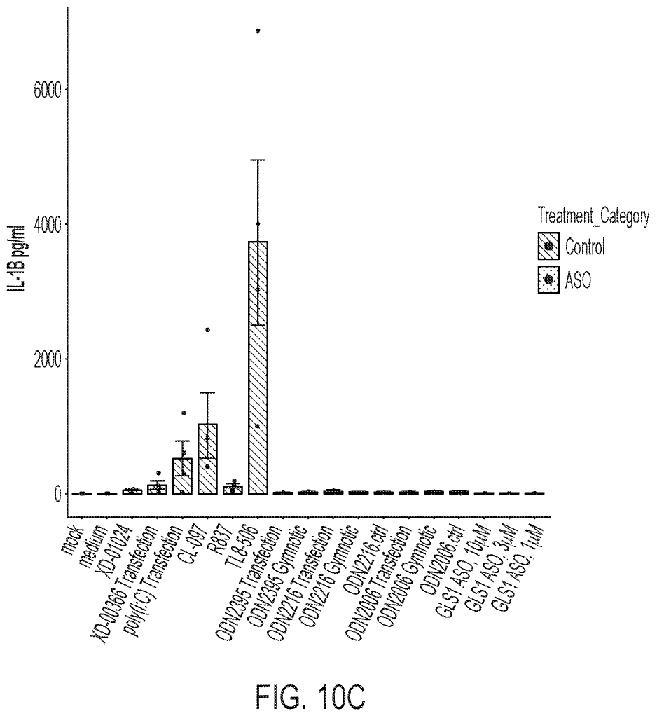

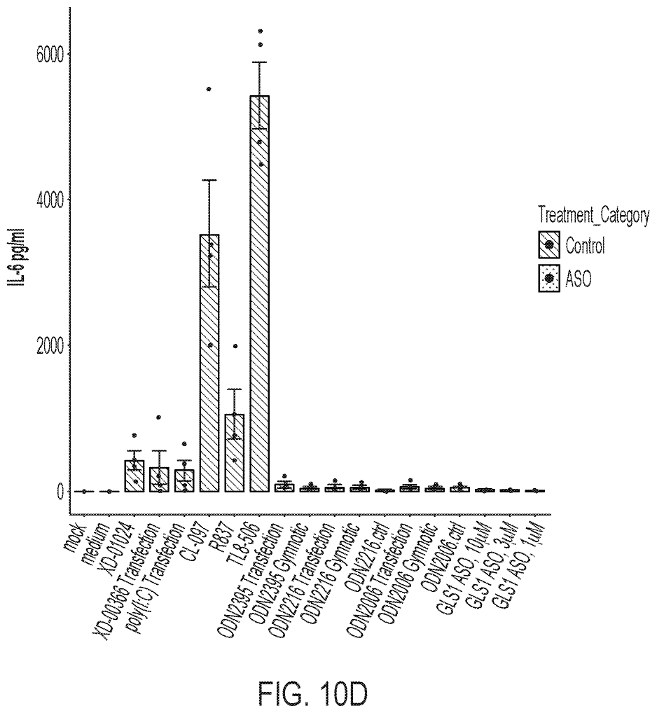

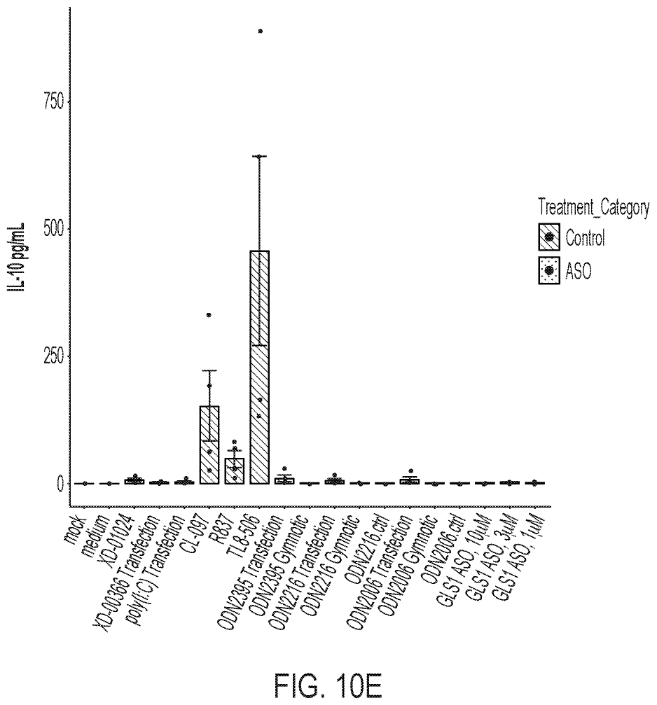

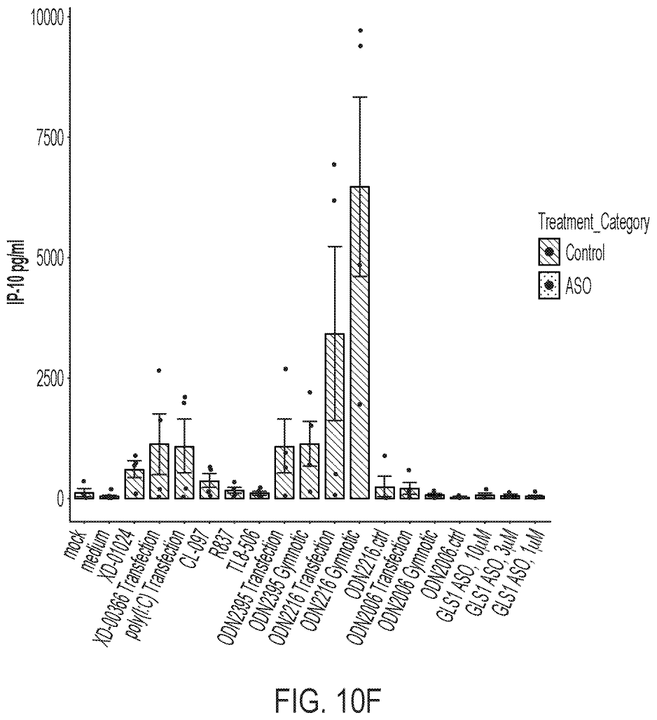

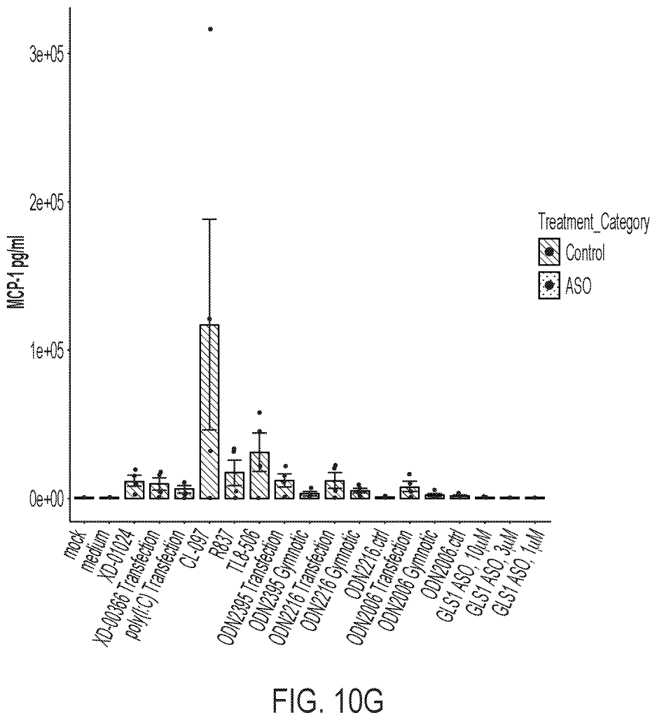

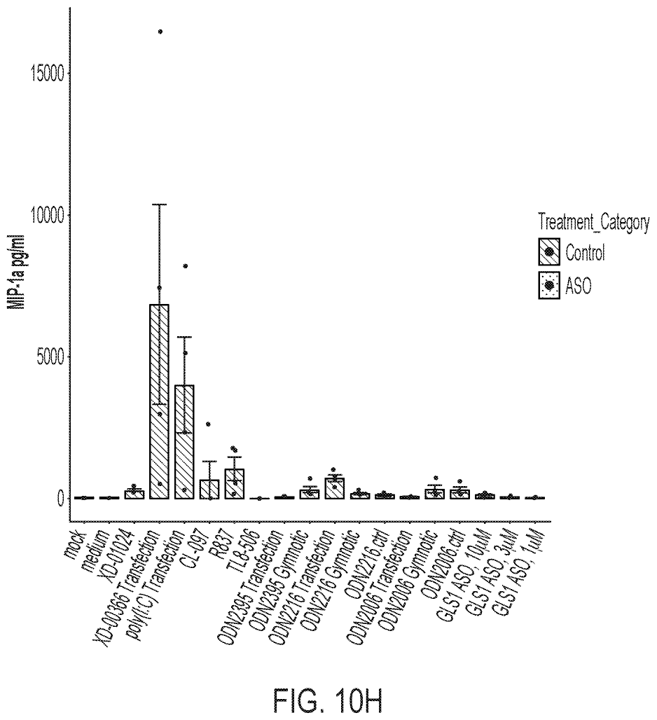

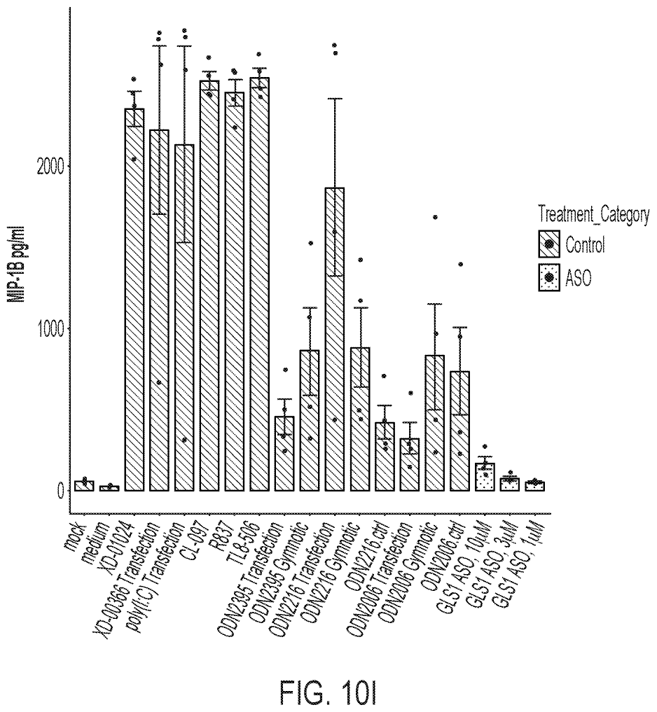

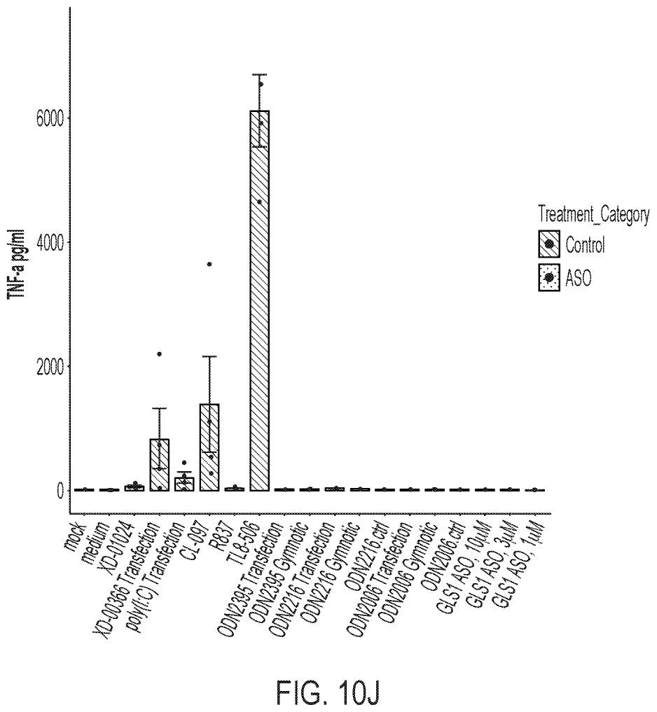

Following treatment of huPBMCs, the levels of IFN-a2a, IFN-b, IL-1B, IL-6, IL-10, IP-10, MCP-1, MIP-1a, MIP-1b and TNF-a were measured using the MSD-U-Plex platform. Representative data from these analyses are shown in FIGS. 10A-10J. Negative control cells exhibited minimal increases or no detectable increase in chemokine/cytokine levels following treatment. Treatment with TLR agonist positive controls resulted in increased chemokine/cytokine levels as expected. When compared relative to cell samples treated under negative or positive control conditions, no immunogenic responses to GLS1 ASO detected (FIGS. 10A-10J).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. An isolated nucleic acid that comprises a region of complementarity with a human GLS1 mRNA transcript, has at least 60% identity to a nucleic acid sequence set forth in any one of SEQ ID NOs: 1-303, and upon binding to the mRNA transcript decreases transcription, splicing, and/or translation of functional glutaminase protein encoded by the mRNA transcript.

2. The isolated nucleic acid of claim 1, wherein the isolated nucleic acid comprises RNA.

3. The isolated nucleic acid of claim 1 or 2, wherein the isolated nucleic acid is an antisense oligonucleotide.

4. The isolated nucleic acid of any one of claims 1 to 3, comprising or consisting of between 10 and 40 nucleotides.

5. The isolated nucleic acid of claim 4, wherein the isolated nucleic acid comprises or consists of between 18 and 25 nucleotides.

6. The isolated nucleic acid of any one of claims 1 to 5, wherein the isolated nucleic acid comprises one or more chemical modifications.

7. The isolated nucleic acid of claim 6, wherein the one or more chemical modifications comprise one or more nucleoside modifications and/or one or more sugar-phosphate backbone modifications.

8. The isolated nucleic acid of claim 7, wherein the one or more nucleoside modifications comprises a 2′-O-methyl (2′-OMe) modification, a 2′-O-MOE modification, a 2′-O-fluoro modification, or a locked nucleic acid (LNA) modification.

9. The isolated nucleic acid of claim 7 or 8, wherein the one or more sugar-phosphate backbone modifications comprises a phosphorothioate backbone modification.

10. The isolated nucleic acid of any one of claims 1 to 9, wherein the isolated nucleic acid is fully chemically modified.

11. The isolated nucleic acid of any one of claims 1 to 10, wherein the isolated nucleic acid comprises one or more deoxyribonucleotides, optionally wherein the isolated nucleic acid is a gapmer.

12. The isolated nucleic acid of any one of claims 1 to 11, wherein the region of complementarity is located in an untranslated region (UTR) of the GLS1 mRNA transcript.

13. The isolated nucleic acid of claim 12, wherein the untranslated region comprises a 5′ UTR, intron, or 3′ UTR of the GLS1 mRNA transcript.

14. The isolated nucleic acid of any one of claims 1 to 11, wherein the region of complementarity is located in a protein coding region of the GLS1 mRNA transcript.

15. The isolated nucleic acid of any one of claims 1 to 11, wherein the region of complementarity is located on an intron-exon boundary of the GLS1 mRNA transcript.

16. The isolated nucleic acid of any one of claims 1 to 15, wherein the region of complementarity comprises at least 10, 11, 12, 13, 14, 15, 16, 17, 18, 19, or 20 continuous nucleotides of the sequence set forth in SEQ ID NO: 304.

17. The isolated nucleic acid of any one of claims 1 to 16, comprising the nucleotide sequence set forth in any one of the nucleotide sequences set forth in Table 1.

18. A composition comprising the isolated nucleic acid of any one of claims 1 to 17, and a pharmaceutically acceptable excipient.

19. A method for reducing glutamate signaling in a cell or a subject, the method comprising administering the isolated nucleic acid of any one of claims 1 to 17 or the composition of claim 18 to a cell or a subject in need thereof.

20. The method of claim 19, wherein the cell is a neuronal cell.

21. The method of claim 19 or 20, wherein the subject comprises one or more mutations in a gene that is associated with a psychiatric disease or disorder, optionally wherein the gene is GLS1.

22. The method of any one of claims 19 to 21, wherein the cell is a human cell, optionally wherein the cell is in a subject.

23. The method of any one of claims 19 to 22, wherein the subject is a human subject.

24. The method of any one of claims 19 to 23, wherein the subject has or is suspected of having a psychiatric disease or disorder, or neuroinflammation.

25. The method of claim 24, wherein the disease or disorder is schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.).

26. The method of any one of claims 19 to 25, wherein the administration is systemic administration, optionally wherein the systemic administration comprises intravenous injection.

27. The method of any one of claims 19 to 25, wherein the administration comprises direct administration to a target tissue of the subject, optionally wherein the direct administration comprises direct injection to the central nervous system (CNS) or direct injection to the peripheral nervous system (PNS).

28. The method of claim 27, wherein the administration comprises placing the subject in a Trendelenburg position during the administration.

29. A method for reducing neuroinflammation in a subject, the method comprising administering the isolated nucleic acid of any one of claims 1 to 17, or the composition of claim 18, to a cell or a subject in need thereof.

30. The method of claim 29, wherein the subject is characterized as not having a mutation in a gene associated with glutamate signaling, optionally wherein the gene is GLS1.

31. The method of claim 29, wherein the subject comprises one or more mutations in a gene that is associated with glutamate signaling, optionally wherein the gene is GLS1.

32. The method of any one of claims 29 to 31, wherein the subject is a human subject.

33. The method of any one of claims 29 to 32, wherein the subject has or is suspected of having a psychiatric disease or disorder.

34. The method of claim 33, wherein the psychiatric disease or disorder is schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.).

35. The method of any one of claims 29 to 34, wherein the administration is systemic administration, optionally wherein the systemic administration comprises intravenous injection.

36. The method of any one of claims 29 to 34, wherein the administration comprises direct administration to the central nervous system (CNS), optionally wherein the direct administration comprises direct injection to the CNS.

37. The method of claim 36 wherein the administration comprises placing the subject in a Trendelenburg position during the administration.

38. A method for preventing or treating a psychiatric disease or disorder in a subject in need thereof, the method comprising administering to the subject the isolated nucleic acid of any one of claims 1 to 17, or the composition of claim 18 to a subject in need thereof.

39. The method of claim 38, wherein the subject is a human.

40. The method of claim 38 or 39, wherein the disease or disorder is schizophrenia (e.g., treatment resistant schizophrenia) and other psychosis (e.g., psychosis associated with dementia, delusional disorder, brief psychotic disorder, etc.), epilepsy (e.g., genetic epilepsy, idiopathic generalized epilepsy, temporal lobe epilepsy, etc.), major depressive disorder, unipolar depression, bipolar disorder, mania, or psychiatric conditions associated with traumatic brain injury, spinal cord injury, ischemic stroke, neuroinflammation, tuberous sclerosis, neurodegenerative disease (e.g., Alzheimer's disease, amyotrophic lateral sclerosis (ALS), motor neuron disease, Huntington's disease, Parkinson's disease, multiple sclerosis, etc.), or neurodevelopmental disorders (e.g., autism, autism spectrum disorder, metabolic encephalopathy, etc.).

41. The method of any one of claims 38 to 40, wherein the administration comprises direct administration to a target tissue of the subject, optionally wherein the direct administration comprises direct injection to the central nervous system (CNS), direct injection to the peripheral nervous system (PNS).
